(19) United States
(12) Patent Application Publication
(10) Pub. No.: US 2024/0076736 A1
(43) Pub. Date: Mar. 7, 2024

(54) COMPOSITIONS AND METHODS FOR CHARACTERIZING POLYNUCLEOTIDE SEQUENCE ALTERATIONS

(71) Applicant: The Brigham and Women's Hospital, Inc., Boston, MA (US)

(72) Inventors: Yuriy BAGLAENKO, Boston, MA (US); Soumya RAYCHAUDHURI, Boston, MA (US)

(73) Assignee: The Brigham and Women's Hospital, Inc., Boston, MA (US)

(21) Appl. No.: 18/494,528

(22) Filed: Oct. 25, 2023

Related U.S. Application Data
(63) Continuation of application No. PCT/US2022/026183, filed on Apr. 25, 2022.
(60) Provisional application No. 63/179,921, filed on Apr. 26, 2021.

(51) Int. Cl. C12Q 1/6874; C12Q 1/6804

(52) U.S. Cl. CPC C12Q 1/6874; C12Q 1/6804; C12Q 1/6844

(57) ABSTRACT

The disclosure provides compositions and methods for characterizing the genome and transcriptome at a single cell level. In some embodiments, the method provides for the characterization of CRISPR editing outcomes and phenotypes, as well as other alterations in polynucleotide sequences, particularly in primary cells.

### CROSS-REFERENCE TO RELATED APPLICATIONS

This application is a continuation under 35 U.S.C. § 111(a) of PCT International Patent Application No. PCT/US2022/026183, filed Apr. 25, 2022, designating the United States and published in English, which claims priority to and the benefit of U.S. Provisional Application No. 63/179,921, filed Apr. 26, 2021, the entire contents of each of which are incorporated by reference herein.

### STATEMENT OF RIGHTS TO INVENTIONS MADE UNDER FEDERALLY SPONSORED RESEARCH

This invention was made with government support under Grant No. AR063759 awarded by the National Institutes of Health. The government has certain rights in the invention.

### SEQUENCE LISTING

The present application contains a Sequence Listing which has been submitted electronically in XML format and is herein incorporated by reference in its entirety. The Sequence Listing XML file, created on Oct. 25, 2023, is named 167741-040002US-Sequence_Listing.xml and is 16,469 bytes in size.

### BACKGROUND OF THE INVENTION

Simultaneous sequencing of the genome and transcriptome at the single-cell level is a powerful tool for characterizing genomic and transcriptomic variation and revealing correlative relationships. However, it remains technically challenging to analyze both the genome and transcriptome in the same cell. Currently, there is a dearth of techniques that allow for the analysis of CRISPR editing outcomes and phenotypes, particularly in primary human cells.

### SUMMARY OF THE INVENTION

As described below, the present disclosure features compositions and methods for characterizing the genome and transcriptome at a single cell level. In some embodiments, the method provides for the characterization of CRISPR editing outcomes and phenotypes using, for example, antibodies for sequencing and hashing from flow cytometry. Similar methods are provided for characterizing other alterations in polynucleotide sequences.

In one aspect, the invention of the disclosure features a method for concurrently characterizing single cell genomic DNA and mRNA. The method involves (a) labelling a plurality of isolated cells with a detectable antibody that specifically binds a cell surface marker of interest. The method also involves (b) incubating the detectably labelled cells of (a) with an oligo-conjugated antibody. The method further involves (c) index sorting the cells into single wells, characterizing the cell surface marker expression of each cell, and lysing the cells in the presence of dNTPs, a well-specific barcoded oligoDT primer containing a unique molecular identifier (UMI), and a PCR handle. The method also involves (d) incubating the product of (c) with reverse transcriptase, a custom template switch oligo (TSO) containing one member of a binding pair, under conditions that permit generation of cDNA. The method further involves (e) incubating the product of step (d) with genomic primers that specifically bind a region of interest (ROI), cDNA amplification primers that specifically bind the PCR handle and the TSO, an antibody derived tag (ADT) specific primer, dNTPs, and a polymerase under conditions that support amplification, thereby simultaneously amplifying gDNA, cDNA, and ADT to form cDNA, genomic ROI, and ADT libraries. The method involves (f) incubating at least a portion of the genomic DNA from each well of (e) with dNTPs, polymerase, and nested primers that specifically bind a region of interest to obtain a gDNA library. At least one of the nested primers contains i) a well-specific barcode, a UMI, and a PCR handle; or ii) a capture sequence. When the nested primers contain the capture sequence, step (e) further involves incubating the product of (e) with an exonuclease and a capture oligo. The capture oligo contains the capture sequence, a well-specific barcode, an exonuclease blocking agent, and a UMI. The capture oligo binds to an amplicon produced using the nested primers effectively labeling the product with the barcode during the PCR reaction. The method also involves (g) pooling at least a portion of a sample from each well after step (e) or step (f), and subsequently separating at least two of the cDNA, ADT libraries, and gDNA libraries. The method also involves (h) preparing the gDNA, cDNA, and ADT libraries for sequencing by amplifying each library in the presence of sequencing primers.

In another aspect, the invention features a method for concurrently characterizing DNA amplicons, 3′ mRNA transcripts, antibody derived tags (ADT), and index flow sorting information from a cell sample. The method involves (a) labelling a plurality of cells with a detectable antibody that specifically binds a cell surface marker of interest and single-cell index sorting the cells into individual wells. The method also involves (b) lysing the cells in the presence of a reverse transcriptase, a template switch oligo, well-specific barcodes, a primer containing an oligoDT primer containing a unique molecular identifier (UMI), and a PCR handle, and ADTs under conditions that permit reverse transcription to obtain cDNA. The method further involves (c) amplifying the cDNA, ADT, and specific genomic DNA in a single pool containing genomic primers that specifically bind a region of interest, cDNA amplification primers that specifically bind the PCR handle and the TSO, an ADT specific primer, dNTPs, and a Taq polymerase, thereby simultaneously amplifying gDNA, cDNA, and ADT to form cDNA, genomic ROI, and ADT libraries. The method further involves (d) at least a portion of the product of (c) is used for further amplification of the genomic ROI with nested primers to obtain a gDNA library. At least one of the nested primers contains i) a well-specific barcode, a UMI, and a PCR handle; or ii) a capture sequence. When the nested primers contain the capture sequence, step (d) further involves incubating the product of (c) with an exonuclease and a capture oligo. The capture oligo contains the capture sequence, a well-specific barcode, an exonuclease blocking agent, and a UMI. The capture oligo binds to an amplicon produced using the nested primers effectively labeling the product with the barcode during the PCR reaction. The method further involves (e) pooling at least a portion of each well and subsequently separating at least two of the gDNA, cDNA, and ADT libraries. The method also involves (f) preparing the gDNA, cDNA, and ADT libraries for sequencing. Preparing the libraries for sequencing involves amplifying the ADT library with sequencing primers, tagmenting the cDNA library and preferentially amplifying the 3′ ends with sequencing primers, and amplifying the gDNA library using sequencing primers.

In another aspect, the invention of the disclosure features a method for concurrently characterizing single cell genomic DNA and mRNA. The method involves (a) labelling a plurality of isolated cells with a detectable antibody that specifically binds a cell surface marker of interest. The method also involves (b) incubating the detectably labelled cells of (a) with an oligo-conjugated antibody. The method further involves (c) index sorting the cells into single wells, characterizing the cell surface marker expression of each cell, and lysing the cells in the presence of dNTPs, a well-specific barcoded oligoDT primer containing a unique molecular identifier (UMI), and a PCR handle, and a capture oligo containing a capture sequence, a well-specific barcode, an exonuclease blocking agent, and a unique molecular identifier. The method further involves (d) incubating the product of (c) with reverse transcriptase, a custom template switch oligo (TSO) containing one member of a binding pair, and a reverse transcriptase under conditions that permit generation of cDNA. The method also involves (e) incubating the product of step (d) with genomic primers that specifically bind a region of interest (ROI), cDNA amplification primers that specifically bind the PCR handle and the TSO, an antibody derived tag (ADT) specific primer, dNTPs, and a polymerase under conditions that support amplification, thereby simultaneously amplifying gDNA, cDNA, and ADT to form cDNA, genomic ROI, and ADT libraries. The method also involves (f) contacting the product of step (e) with an exonuclease to degrade unconsumed primers. The method further involves (g) incubating at least a portion of the genomic ROI libraries from each well of (f) with dNTPs, polymerase, and nested primers capable of specific amplification of a region within the genomic ROI library. At least one of the nested primers contains the capture sequence. The capture oligo binds to an amplicon produced using the nested primers effectively labeling the product with the barcode during the PCR reaction, and obtaining a gDNA library. The method also involves (g) pooling at least a portion of a sample from each well, and subsequently separating the gDNA, cDNA, and ADT libraries. The method further involves (h) preparing the gDNA, cDNA, and ADT libraries for sequencing by amplifying each library in the presence of sequencing primers.

In another aspect, the invention of the disclosure provides a method for concurrently characterizing single cell genomic DNA and mRNA. The method involves (a) labelling a plurality of isolated cells with a detectable antibody that specifically binds a cell surface marker of interest. The method further involves (b) incubating the detectably labelled cells of (a) with an oligo-conjugated antibody. The method also involves (c) index sorting the cells into single wells, characterizing the cell surface marker expression of each cell, and lysing the cells in the presence of dNTPs, and a well-specific barcoded oligoDT primer containing a unique molecular identifier (UMI), and a PCR handle. The method further involves (d) incubating the product of (c) with reverse transcriptase, and a custom template switch oligo (TSO) containing one member of a binding pair, under conditions that permit generation of cDNA. The method further involves (e) incubating the product of step (d) with genomic primers that specifically bind a region of interest (ROI), cDNA amplification primers that specifically bind the PCR handle and the TSO, an antibody derived tag (ADT) specific primer, dNTPs, and a polymerase under conditions that support amplification, thereby simultaneously amplifying gDNA, cDNA, and ADT to form cDNA, genomic ROI, and ADT libraries. The method also involves (f) pooling at least a portion of a sample from each well, and subsequently separating the cDNA and ADT libraries. The method also involves (g) incubating at least a portion of the genomic DNA from each well of (e) with dNTPs, polymerase, and nested primers that specifically bind a region of interest to obtain a gDNA library. The nested primers contain a well-specific barcode, a UMI, and a PCR handle. The method involves (h) preparing the gDNA, cDNA, and ADT libraries for sequencing by amplifying each library in the presence of sequencing primers.

In any of the above aspects, or embodiments thereof, the method further involves sequencing the libraries.

In any of the above aspects, or embodiments thereof, the method further involves adding the capture oligo prior to amplification of the gDNA, cDNA, and ADT for the first time.

In any of the above aspects, or embodiments thereof, the exonuclease is ExoI. In any of the above aspects, or embodiments thereof, the blocking agent is a phosphoryl or acetyl group. In any of the above aspects, or embodiments thereof, the blocking agent is linked to the 3′OH group of the capture oligomer.

In any of the above aspects, or embodiments thereof, all amplifications prior to preparing the gDNA, cDNA, and ADT libraries are carried out in the same well. In any of the above aspects, or embodiments thereof, formation of the cDNA, genomic ROI, and ADT libraries is carried out in a first well and the gDNA library is prepared in a separate well.

In any of the above aspects, or embodiments thereof, the gDNA, cDNA, and/or ADT libraries are separated using Solid Phase Reversible Immobilization beads (SPRI) beads.

In any of the above aspects, or embodiments thereof, the separation involves first separating the gDNA library from the cDNA and ADT libraries using SPRI beads and subsequently separating the cDNA library from the ADT library using SPRI beads. In any of the above aspects, or embodiments thereof, the separation of the cDNA library from the ADT library involves separating from one another amplicons that are greater than 500 bp in length and amplicons that are less than 500 bp in length, respectively. In any of the above aspects, or embodiments thereof, where separation of the cDNA and ADT libraries is carried out prior to or in parallel with preparation of the gDNA library.

In any of the above aspects, or embodiments thereof, one or more of the cells contains an alteration in a genomic DNA sequence relative to the sequence of a reference genome. In embodiments, the alteration was introduced using a genomic editing technique. In embodiments, the genomic editing technique involves base-editing or homology-directed recombination (HDR) editing.

In any of the above aspects, or embodiments thereof, one or more of the cells contains an alteration in mRNA expression relative to the mRNA expression of a reference cell. In any of the above aspects, or embodiments thereof, one or more of the cells contains an alteration in the expression of a cell surface marker relative to a reference cell.

In any of the above aspects, or embodiments thereof, the cells are edited using CRISPR prior to characterization. In any of the above aspects, or embodiments thereof, the cells are primary cells. In any of the above aspects, or embodiments thereof, the cells are immune cells. In any of the above aspects, or embodiments thereof, the cells are mammalian cells. In any of the above aspects, or embodiments thereof, the cells are human cells.

In any of the above aspects, or embodiments thereof, the cells are sorted using a FACS sorter. In any of the above aspects, or embodiments thereof, at least about 500,000 to more than ten million cells are characterized. In any of the above aspects, or embodiments thereof, the cell surface marker is CD45, CD81, or MHC class 1.

In any of the above aspects, or embodiments thereof, the polymerase is a Taq polymerase. In embodiments, the Taq polymerase is KAPA HiFI Taq polymerase or Q5 Taq polymerase.

In any of the above aspects, or embodiments thereof, after an incubation or amplification step the product of the incubation or amplification is cleaned. In embodiments, the cleaning is carried out using Solid Phase Reversible Immobilization beads (SPRI) beads.

In any of the above aspects, or embodiments thereof, the detectable antibody contains a fluorphore. In any of the above aspects, or embodiments thereof, the oligo-conjugated antibody contains a poly-A sequence.

In any of the above aspects, or embodiments thereof, the sequencing primers are Illumina primers P5 and P7.

In any of the above aspects, or embodiments thereof, steps (c) to (e) happen concurrently or sequentially.

Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The term “adaptor” refers a sequence that is added, for example by ligation, to a nucleic acid. The length of an adaptor may be from about 5 to about 100 bases, and may provide a sequencing primer binding site (e.g., an amplification primer binding site), and a molecular barcode such as a sample identifier sequence or molecule identifier sequence, preferably a unique identifier sequence. An adaptor may be added to 1) the 5′ end, 2) the 3′ end, or 3) both ends of a nucleic acid molecule. Double-stranded adaptors contain a double-stranded end ligated to a nucleic acid. An adaptor can have an overhang or may be blunt ended. As will be described in greater detail below, a double stranded adaptor can be added to a fragment by ligating only one strand of the adaptor to the fragment. The sequence of the non-ligated strand of the adaptor may be added to the fragment using a polymerase. Y-adaptors and loop adaptors are type of double-stranded adaptors.

By “alteration” is meant a change (increase or decrease) in the structure, expression levels or activity of a gene or polypeptide as detected by standard art known methods such as those described herein. In one embodiment, a change in sequence (i.e., insertion, deletion, point mutation, copy number alteration (CNA), or loss in heterozygosity (LOH) is determined relative to a reference sequence, reference exome, and/or reference genome. In some embodiments, the alteration is an alteration in the sequence of a polynucleotide, for example, an alteration associated with CRISPR editing. As used herein, an alteration includes a 10% change in expression levels, preferably a 25% change, more preferably a 40% change, and most preferably a 50% or greater change in expression levels.

By “amplicon” is meant a piece of a nucleic acid such as for example, DNA or RNA, that is the source and/or product of amplification or replication.

As used herein, the term “antisense strand” refers to a polynucleotide that is substantially or 100% complementary to a target nucleic acid of interest. For example, an antisense strand may be complementary, in whole or in part, to a molecule of mRNA (messenger RNA), an RNA sequence that is not mRNA (e.g., microRNA, piwiRNA, tRNA, rRNA and hnRNA) or a sequence of DNA that is either coding or non-coding. The terms “antisense strand” and “guide strand” are used interchangeably herein.

“Biological sample” as used herein refers to a sample obtained from a biological subject, including a sample of biological tissue or fluid origin, obtained, reached, or collected in vivo or in situ, that contains or is suspected of containing polynucleotides. A biological sample also includes samples from a region of a biological subject containing immune cells, precancerous or cancer cells or tissues. Such samples can be, but are not limited to, organs, tissues, fractions and cells isolated from mammals including, humans such as a patient, mice, and rats. Biological samples also may include sections of the biological sample including tissues, for example, frozen sections taken for histologic purposes.

By “barcode” is meant a degenerate or semi-degenerate nucleic acid sequence that varies plasmid to plasmid or genome to genome. The barcode sequence may be a degenerate or a semi-degenerate sequence that is identifiable. For example, the barcodes may comprise identifiable degenerate sequences that have several possible bases in any of the positions of the nucleic acid sequence. A barcode may uniquely label or detect a single cell. A barcode may also be used in sequencing to identify a genome.

By “complementary” is meant capable of pairing to form a double-stranded nucleic acid molecule or portion thereof. The complementarity need not be perfect, but may include mismatches at 1, 2, 3, or more nucleotides.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected. In some embodiments, the analyte is a sequence alteration.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “exonuclease” is meant an enzyme that cleaves a polynucleotide chain from the end of the chain by removing the nucleotides one by one. In an embodiment, an exonuclease useful for selectively degrading linear DNA, as opposed to circular DNA, is RecBCD.

The term “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989 Molecular Cloning: A Laboratory Manual, 18.1-18.88). Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are utilized during the transcription and the translation of a gene. Further, a “protein gene product” is a protein expressed from a particular gene.

By “genomic library” is meant an entire genome of an organism, virus, bacteria, plant, or cell, or a collection of cloned DNA molecules consisting of at least one copy of every gene from a particular organism or cell.

By “high-throughput sequencing” is meant a sequencing technique that allows for large amounts of nucleic acids to be sequenced.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with an alteration in the genome of a cell, or a disease or disorder.

As used herein, “obtaining” as in “obtaining an agent” includes synthesizing, purchasing, or otherwise acquiring the agent.

“Primer set” means a set of oligonucleotides that may be used, for example, for PCR. A primer set would consist of at least 2, 4, 6, 8, 10, 12, 14, 16, 18, 20, 30, 40, 50, 60, 80, 100, 200, 250, 300, 400, 500, 600, or more primers.

By “reduces” is meant a negative alteration of at least 10%, 25%, 50%, 75%, or 100%.

By “reference” is meant a standard or control condition.

A “reference genome” is a defined genome used as a basis for genome comparison or for alignment of sequencing reads thereto. A reference genome may be a subset of or the entirety of a specified genome; for example, a subset of a genome sequence, such as exome sequence, or the complete genome sequence.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “RNA-seq” is meant RNA sequencing for detecting and quantifying messenger RNA molecules (mRNA) in a biological sample, which, for example, may be used to study cellular responses. A related term, “scRNA-seq” is single-cell RNA sequencing, which may be, for example, a droplet-based single-cell RNA-seq or “Drop-seq,” that is a sequencing technology for analyzing RNA expression in at least hundreds of thousands of individual cells in embodiments of the invention, but may alternatively use any other high-throughput sequencing platform.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

The term “tagmentation” refers to a step in the Assay for Transposase Accessible Chromatin using sequencing (ATAC-seq) as described. (See, Buenrostro, J. D., Giresi, P. G., Zaba, L. C., Chang, H. Y., Greenleaf, W. J., Transposition of native chromatin for fast and sensitive epigenomic profiling of open chromatin, DNA-binding proteins and nucleosome position. Nature methods 2013; 10 (12): 1213-1218). Specifically, a hyperactive Tn5 transposase loaded in vitro with adapters for high-throughput DNA sequencing, can simultaneously fragment and tag a genome with sequencing adapters. In one embodiment the adapters are compatible with the methods described herein.

Single-cell ATAC-seq detects open chromatin in individual cells. ATAC-seq (assay for transposase-accessible chromatin) identifies regions of open chromatin using a hyperactive prokaryotic Tn5-transposase, which preferentially inserts into accessible chromatin and tags the sites with sequencing adaptors (Buenrostro J D, Giresi P G, Zaba L C, Chang H Y, Greenleaf W J. Transposition of native chromatin for fast and sensitive epigenomic profiling of open chromatin, DNA-binding proteins and nucleosome position. Nat Methods. 2013; 10:1213-128). The protocol is straightforward and robust and has become widely popular. Up to this point, ATAC-seq and other methods for the identification of open chromatin have required large pools of cells (Buenrostro, 2013; Thurman R E, Rynes E, Humbert R, Vierstra J, Maurano M T, Haugen E, et al. The accessible chromatin landscape of the human genome. Nature. 2012; 488:75-82), meaning that the data collected reflect cumulative accessibility across all cells in the pool. Independent studies have modified the ATAC-seq protocol for application to single cells (scATAC-seq) (Buenrostro J D, Wu B, Litzenburger U M, Ruff D, Gonzales M L, Snyder M P, et al. Single-cell chromatin accessibility reveals principles of regulatory variation. Nature. 2015; 523:486-90; and Cusanovich D A, Daza R, Adey A, Pliner H A, Christiansen L, Gunderson K L, et al. Epigenetics. Multiplex single-cell profiling of chromatin accessibility by combinatorial cellular indexing. Science. 2015; 348:910-4). These studies provide data on hundreds (Buenrostro, 2015) or thousands (Cusanovich, 2015) of single cells in parallel. Both methods are limited in either the number of cells analyzed or the per-cell coverage.

By “transcriptome” is meant all of the messenger RNA (mRNA) molecules expressed from the genes of an organism's RNA.

By “unique molecular identifier” or “UMI” is meant short nucleic acid sequence that is identifiable in, for example, high-throughput sequencing techniques, such as but not limited to single-cell RNA-seq. The UMIs may be used to not only detect, but also to quantify.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1G provide schematics, boxplots, plots, and a heatmap showing multi-omic single cell analysis of genomic DNA and mRNA from CRISPR-editedHH cells identified a strong correlation between induced deletion size and HLADQB1 expression. FIG. 1A provides a representative schematic of multi-omic single cell editing of HH cells. FIG. 1B provides a representative plot of exons in HLADQB1 and the location of the sgRNA. Example amplicon alignment to reference sequence from single HH cells generated with CRISPResso2. FIG. 1C provides a heatmap of single cell DNA editing with each row representing a cell and each column a nucleotide. Cells are shaded by the percentage of reads with any modification (insertion, deletion, or substitution) at that nucleotide with colors representing different cell clusters. Grouping of cells were identified with defined k-means clustering. FIG. 1D provides a boxplots of single cell HLADQB1 gene expression per DNA cluster defined in FIG. 1C. FIGS. 1E-1F provide plots showing correlations of average deletion size to HLADQB1 and HLADRB1 gene expression. Correlations were calculated using linear regression models with p-values of gene coefficients shown. FIG. 1G provides a Manhattan plot of genome wide differential gene (non-zero in 30% of cells) expression analysis was performed with DESeq2 with deletion size as the response variable. The red line represents a Bonferroni corrected p-value of 0.01. Each dot represents a single cell. Gene expression values are scaled and normalized with Seurat.

FIGS. 2A-2K provide a schematic, flow cytometry plots, diagrams, boxplots, a volcano plot, and scatter plots showing single cell multi-omic sequencing of PTPRC base-edited primary CD4 T cells identified robust correlations between distinct genotypes and protein expression. FIG. 2A provides a schematic of base-editing an early stop codon in PTRPC in primary CD4 T cells. Grey filled circles represent terminal bulk data collection points. Black filled circles represent terminal single cell collection points. FIG. 2A discloses SEQ ID NOs: 3, 4 and 4, respectively, in order of appearance. FIG. 2B provides representative flow cytometry plots and summary analysis of non-targeting (N) and base-edited knockout samples (BE). The red line indicates the samples used for single cell MINECRAFTseq processing. Significant was measured using a paired Mann-Whitney U test (** p<0.01). FIG. 2C provides a diagram showing bulk DNA editing results from three healthy individual samples with the targeted nucleotide highlighted. The arrow indicates the location of the sgRNAaway from the PAM. FIG. 2C discloses SEQ ID NO: 5. FIG. 2D provides boxplots (left panel) showing bulk mRNA expression of PTPRC from 4 healthy individuals. Gene expression values are scaled and normalized as log UMI+1. FIG. 2D also provides a volcano plot (right panel) of differential gene expression with each dot representing a tested gene. The solid line represents the Bonferroni corrected p-value of 0.01. FIG. 2E provides a diagram. FIG. 2E discloses two instances of SEQ ID NO: 5. 10 plates from 1 individual (light grey line in FIG. 2B) were processed with a single cell MINECRAFTseq protocol. Recovered common genotypes (greater than 4 cells). Rare genotypes (less than or equal to 4 cells) are not shown. Histogram and numbers on the right hand side represent the number of cells from each genotype. The arrow indicates the sequence and location of the sgRNA, pointing away from the PAM site. FIG. 2F provides boxplots of corresponding expression of CD45-FITC as measured by index flow cytometry and bi-exponentially scaled or CLR normalized ADT counts of CD45 (FIG. 2G) from each genotype in FIG. 2E. FIG. 2H provides a plot showing Uniform Manifold Approximation and Projection (UMAP) of all 33 measured ADT markers colored by CD45 expression. FIG. 2I provides boxplots showing all significant changes in ADT markers correlated to dosage at the targeted base (comparing genotypes A, C & B). All other genotypes were excluded from the analysis. CLR normalized counts of each marker. ADT markers are ordered by average expression. FIG. 2J provides a plot showing Uniform Manifold Approximation and Projection (UMAP) of variable gene expression from single cells with color representing scaled and normalized expression of PTPRC. FIG. 2K provides boxplots of gene expression of PTPRC by genotype with genotypes D-H grouped into a single category. Unless otherwise specified, each dot represents a cell.

FIGS. 3A-3K provide a schematic, diagrams, heat maps, volcano plots, and boxplots showing genomic editing of four variants in the UBASH3A autoimmune locus with single cell MINECRAFTseq identifies causal variants. Three healthy individuals, all reference for the four targeted variants, were recruited, CD4 T cell isolated, and genomically edited at the individual variants of interest. HDR or BE samples and controls were indexed, pooled, and multi-omic single cell libraries prepared as shown in FIGS. 8A and 8B. In some embodiments, libraries are prepared as shown in FIGS. 19A and 19B. FIG. 3A provides a Schematic of the UBASH3A locus with variants of interest highlighted along with the CRISPR-Cas editing technology used for investigation. Recovered common genotypes (greater than 4 cells) with sgRNA sequence and cell numbers indicated for targeting of (FIG. 3B) rs80054410 and (FIG. 3C) rs11203203. FIG. 3B discloses two instances of SEQ ID NO: 6. FIG. 3C discloses two instances of SEQ ID NO: 7. Heatmaps of editing at the (FIG. 3D) rs9981624 and (FIG. 3E) rs11203202 loci with locations of variants and sgRNA indicated (arrow pointing away from the PAM). Each row represents a cell and each column a nucleotide in the amplicon sequence. Shading represents the percentage of reads edited (insertion, deletion, or substitution) at the nucleotide. DNA editing clusters were defined with k-means clustering assuming four clusters. Volcano plots of differential gene (greater than 30% non-zero) expression to dosage (0,1,2) at (FIG. 3F) rs80054410 and (FIG. 3G) rs11203203 accounting for plate. Each dot represents a gene. FIG. 3H provides a plot of RIPK1 scaled and normalized gene expression per genotypes identified in FIG. 3C. Volcano plots of differential gene (greater than 30% non-zero) expression to average deletion size at (FIG. 3I) rs9981624 and (FIG. 3J) rs11203202 accounting for plate. Each dot represents a gene. FIG. 3K provides a plot showing IL2RA scaled and normalized gene expression per DNA clusters identified in FIG. 3E. Differential gene expression was performed with DESeq2 on unscaled and unnormalized values. Solid lines on volcano plots are Bonferroni corrected p values of 0.05. For H and K, each dot represents a cell. Scaled and normalized gene expression was calculated with Seurat.

FIGS. 4A-4I provide a schematic, diagrams, boxplots, and volcano plots showing CRISPR-Cas base-editing of three variants in IL2RA confirmed causality in rs61839660 and a nearby nucleotide in regulating CD25 expression. Three healthy individuals, with different genotypes at the three targeted variants, were recruited, CD4 T cell isolated, and genomically edited at the individual variants of interest or as a large multiplex pool. All conditions were indexed, pooled, and multi-omic single cell libraries prepared as shown in FIGS. 8A and 8B. In some embodiments, libraries are prepared as shown in FIGS. 19A and 19B. Sequences from each variant in each cell were generated. FIG. 4A provides a schematic of the IL2RA locus with variants of interest highlighted. FIG. 4B provides a diagram showing the conditions used in this experiment with different CRISPR-Cas base-editors. FIG. 4B discloses “TTTTTTTTTTTTT” as SEQ ID NO: 3. FIG. 4C provides a diagram showing recovered very common genotypes (greater than 20 cells for brevity) with sgRNA sequence and cell numbers indicated (right) for all three targeted regions. Location of variants of interest along with the named “multiplex SNP”. FIG. 4C discloses two instances of SEQ ID NOs: 8-10 each, respectively, in order of appearance. Bottom induced single-nucleotide polymorphism (SNP) ids (SNP1-18) are named on the bottom and represent the location of any identified mutation in the study used in follow-up analysis. A full breakdown on per individual and per condition genotypes can be found in FIGS. 18A and 18B. FIG. 4D provides boxplots showing CLR normalized counts of ADT markers significant for dosage at the identified multiplexSNP. FIG. 4E provides boxplots showing CLR normalized counts of ADT markers significant for dosage at rs61839660 conditioning on the multiplex SNP. FIG. 4F provides Volcano plots of differential gene (greater than 30% non-zero) expression to dosage at the rs61839660 correcting for plate and dosage at the multiplex SNP. FIG. 4H provides a volcano plot showing RORA scaled and normalized gene expression per dosage at rs61839660 regardless of genotype and faceted by individual. Volcano plots of differential gene (greater than 30% non-zero) expression to dosage at the multiplex SNP accounting for plate. FIG. 4I provides boxplots showing MAPK6 scaled and normalized gene expression per dosage at the multiplex SNP regardless of genotype and faceted by individual. Differential gene expression was performed with DESeq2 on unscaled and unnormalized values. Solid lines on volcano plots are Bonferroni corrected p values of 0.05. For volcano plots, each dot represents a gene. In FIGS. 4G and 4I, each dot represents a cell. Scaled and normalized gene expression was calculated with Seurat.

FIGS. 5A-5E provide violin plots, Uniform Manifold Approximation and Projection (UMAP) plots, a heat map, and boxplots showing genomic DNA amplicon metrics from HH edited cells. FIG. 5A provides violin plots showing all editing (substitutions, insertions, and deletions) as a ratio of edited reads from 0 to 1 summed across all examined cells and graphed on a per nucleotide basis across the amplicon. Each dot represents a nucleotide in the amplicon and the peak indicates the center of the CRISPR edit and the area most likely mutated. Values were extracted from a CRISPResso2 analysis as described in the methods. FIG. 5D provides a heatmap showing number of aligned reads to the indicated amplicons per cell. FIGS. 5B and 5C provide Uniform Manifold Approximation and Projection (UMAP) plots. FIG. 5E provides boxplots showing HLA-DQB1 gene expression.

FIGS. 6A-6G provide a schematics, boxplots, and a diagram showing optimizations of multi-omic single cell protocols to capture genomic DNA, ADT, and mRNA from Jurkat cells base-edited at variant rs61839660. FIG. 6A provides a schematic of experimental outline and a schematic of the IL2RA locus and targeted variant (rs61839660). FIG. 6A discloses SEQ ID NOs: 3, 4 and 4, respectively, in order of appearance. FIG. 6B provides a boxplots of total genomic DNA reads recovered per cell and percentage of reads edited at the targeted base per cell per condition defined in A. FIG. 6C provides boxplots of total antibody derived tags (ADT) unique molecular identifiers (UMIs) recovered per cell and distributions of count log ratio normalized counts of each antibody. FIG. 6D provides boxplots showing UMIs per cell and total number of genes recovered per cell per condition. In FIGS. 6A-6D, all comparisons between conditions are significant using a Kruskal-Wallis test with Dunn's post-test comparison. FIG. 6E provides a diagram showing recovered common genotypes (greater than 4 cells). Rare genotypes (less than or equal to 4 cells) are not shown. Histogram and numbers on the right hand side represent the number of cells from each genotype. The arrow indicates the sequence and location of the sgRNA, pointing away from the PAM site. FIG. 6E discloses two instances of SEQ ID NO: 11. FIG. 6F provides boxplots showing gene expression of IL2RA and CLR normalized counts of ADT CD25 in G1, G2, G3, and G4+ based on genotypes in E. G4+ represents genotype G4 and all other rare genotypes with less than 4 cells. FIG. 6G provides a volcano plot and boxplots showing differential gene (non-zero in 30% of cells) expression to dosage at the targeted variant (G1, G2, G3) excluding all rare (G4+) genotypes. In FIG. 6G, each dot represents a gene. The dotted line is the Bonferroni corrected p-value of 0.05. Expression of the significant gene in all four genotypes is shown. Each dot represents a cell. Gene expression values were scaled and normalized with Seurat.

FIGS. 7A-7D provide plots and a heatmap showing RNA clustering of CRISPR-Cas edited Jurkats. FIG. 7A provides a plot showing an analysis of RNA from rs61839660 edited Jurkats as described in FIGS. 2A-2K using Seurat where 6 clusters were identified. FIG. 7B provides a plot showing RNA clustering did not reveal any bias by condition after implementation of Harmony. FIG. 7C provides a plot showing that IL2RA gene expression was not significantly different per cluster. FIG. 7D provides a heatmap showing log FC genes identified in a differential gene expression analysis with Poisson modelling for RNA clusters. Each dot represents a cell.

FIGS. 8A and 8B provide schematics of single cell MINECRAFTseq. FIG. 8A provides a schematic showing an overview of CD4T cell isolation, CRISPR-editing, indexing, ADT staining, and sorting of cells prior to library generation. FIG. 8A discloses SEQ ID NOs: 3, 4, 4, 4 and 4, respectively, in order of appearance. FIG. 8B provides a schematic showing an overview of library generation for sequencing from each of the three single cell modalities, genomic DNA (top of rightmost portion of the figure), mRNA (middle of rightmost portion of the figure), and antibody derived tags (ADT, bottom of rightmost portion of the figure).

FIGS. 9A-9C provide plots and histograms showing ADT metrics and correlation to index flow cytometry information from PTRPC edited primary CD4 T cells. FIG. 9A provides a Uniform Manifold Approximation and Projection (UMAP) of ADT markers is well mixed by plate. FIG. 9B provides a plot of index flow staining of CD45-FITC (biexponentially transformed values on they-axis) and CLR normalized counts of ADT UMIs (x-axis) were strongly correlated and identified knockouts, heterozygotes, and wildtype cells. Genotypes (A,B,C) of cells are defined in FIGS. 3B and 3C. FIG. 9C provides histograms showing CLR normalized counts of all 33 ADT markers used in the experiment from all cells. Each dot represents a cell.

FIGS. 10A-10D provide violin plots, plots, and a heatmap showing RNA metrics and clustering of PTRPC edited primary CD4 T cells. FIG. 10A provides violin plots showing percent of mitochondrial reads, number of unique molecular identifiers (UMIs) and the total number of genes detected per cell. Cells were not filtered on any criteria before plotting. FIG. 10B provides a Uniform Manifold Approximation and Projection (UMAP) plot based on variable gene mRNA PCs with clusters identified in Seurat. FIG. 10C provides an RNA UMAP plot with plate identity plotted. FIG. 10D provides a heatmap showing log FC genes identified in a differential gene expression analysis with Poisson modelling for RNA clusters. Each dot represents a single cell. RNA analysis was performed in Seurat.

FIGS. 11A-11D provide a volcano plot and boxplots showing differential gene expression of PTRPC edited primary CD4 T cells. FIG. 11A provides a volcano plot of differential gene expression to dosage at the targeted nucleotide. Only genotypes A, B, and C defined in FIGS. 3B and 3C were used in the analysis. Genes in the analysis were selected based on greater than 30% non-zero expression. Dashed line on the volcano plot is the Bonferroni corrected p values of 0.05. Each dot represents a gene. FIGS. 11B-11D provide boxplots showing scaled and normalized gene expression of the top three identified genes in FIG. 11A. Each dot represents a cell. Scaled and normalized gene expression was calculated with Seurat.

FIGS. 12A-12J provide diagrams, boxplots, and plots showing bulk RNA, DNA, and flow cytometry data from editing in the UBASH3A locus. FIGS. 12A-12D provide diagrams showing bulk DNA editing results from three healthy individuals with the targeted nucleotide or region highlighted generated using CRISPResso2. The arrow indicates the location of the sgRNA away from the PAM. N is the non-targeting, HDR is homology directed repair, BE is base-edited samples. Numbers indicate percentage of read modified with black bars signifying deletions. FIG. 12A discloses SEQ ID NO: 12. FIG. 12B discloses SEQ ID NO: 13. FIG. 12C discloses SEQ ID NO: 6. FIG. 12D discloses SEQ ID NO: 7. FIGS. 12E-12J provide boxplots and plots showing bulk mRNA expression of UBASH3A from the same healthy individuals. Gene expression values are scaled and normalized as log UMI+1. (G&H) Bulk flow cytometry from the sample healthy individuals is shown as median fluorescence intensity of key immune markers on CD4 T cells. Flow cytometry values were calculated with FlowJo and plotted using GraphPad. Dots connected by lines are from paired samples.

FIGS. 13A-13D provide diagrams and bar graphs presenting sequence data relating to HDR corrected cells from rs11203202 and rs9981624 editing conditions. FIGS. 13A and 13B provide diagrams showing recovered corrected genotypes with sgRNA sequence and cell numbers indicated (right). FIG. 13A discloses two instances of SEQ ID NO: 13. FIG. 13B discloses two instances of SEQ ID NO: 12. Number of cells with specific insertion (− value) or deletion (+ value) for (FIG. 13C) rs11203202 HDR edited or (FIG. 13D) rs9981624 HDR edited samples. Most edited cells from the rs11203202 contained a single insertion as evident from the bulk data.

FIGS. 14A-14L provide heatmaps, violin plots, and plots showing single cell RNA metrics and clustering from editing variants in the UBASH3A locus. FIG. 14D provides a violin plot showing percent of mitochondrial reads, number of unique molecular identifiers (UMIs) and the total number of genes detected per cell from base-edited cells including non-targeting control, rs80054410, and rs11203203 conditions. FIG. 14E provides a Uniform Manifold Approximation and Projection (UMAP) plot based on variable gene mRNA PCs with clusters identified in Seurat from base-edited cells. FIG. 14F provides an RNA UMAP with expression of UBASH3A plotted from base-edited cells. FIG. 14A provides a heatmap showing log FC genes identified in a differential gene expression analysis with Poisson modelling for RNA clusters from base-edited cells. FIG. 14J provides a violin plot showing percent of mitochondrial reads, number of unique molecular identifiers (UMIs) and the total number of genes detected per cell from HDR-edited cells including non-targeting control, rs11203202, and rs9981624 conditions. FIG. 14K provides a UMAP plot based on variable gene mRNA PCs with clusters identified in Seurat from HDR edited cells. FIG. 14L provides an RNA UMAP with expression of UBASH3A plotted from HDR edited cells. FIG. 14G provides a heatmap showing log FC genes identified in a differential gene expression analysis with Poisson modelling for RNA clusters from HDR-edited cells. FIGS. 14B, 14C, which both relate to rs11203203, and 14H, and 14I, which both relate to rs9981624, provide violin plots showing #UMIs and #Genes. Each dot represents a single cell. RNA analysis was performed in Seurat.

FIGS. 15A-15N provide histograms and plots showing that editing variants in the UBASH3A locus did not impact on cell surface protein expression. FIG. 15A provides histograms showing CLR normalized distribution of all measured ADT markers from base-edited samples. FIGS. 15B-15D provide plots showing expression of HLA-DR, CD27, and CD45RO delineate distinct clusters of CD4 T cells in base-edited samples. FIGS. 15E-15G provide plots showing that cells were equally mixed by donor, plate, and condition in base edited samples. FIG. 15H provides histograms showing CLR normalized distribution of all measured ADT markers from HDR edited samples. FIGS. 15I-15K provide plots showing that expression of HLA-DR, CD27, and CD45RO form delineate distinct clusters of CD4 T cells in HDR edited samples. FIGS. 15L-15N provide plots showing that Cells were equally mixed by donor, plate, and condition in base edited samples. Each dot represents a single cell.

FIGS. 16A-16C provide diagrams, a histogram, and plots showing bulk RNA, DNA, and flow cytometry data from editing in the Il2RA locus. FIG. 16A provides a diagram showing bulk DNA editing results from three healthy individuals with the targeted nucleotide or region highlighted generated using CRISPResso2. The arrow indicates the location of the sgRNA away from the PAM. N is the non-targeting, BE is individually base-edited samples and Multiplex is simultaneous editing at all three variants. Numbers indicate percentage of read modified. FIG. 16A discloses SEQ ID NOs: 8-10, respectively, in order of appearance. FIG. 16B provides an overlay of flow cytometry histograms and a plot showing representative bulk flow cytometry from edited samples and median fluorescence intensity relative to control. Flow cytometry values were extracted with FlowJo and plotted using GraphPad. FIG. 16C provides plots showing bulk mRNA expression of IL2RA from the same healthy individuals. Gene expression values are scaled and normalized as log UMI+1. Dots connected by lines indicate paired samples.

FIG. 17 provides diagrams showing single cell DNA genotypes from each individual per condition from editing in the IL2RA locus. An expanded view of the common (greater than 4 cells) genotypes identified per individual and condition. Cell numbers per genotype are indicated on the right of each plot. Individuals are in columns and conditions in rows. FIG. 17 discloses two instances of SEQ ID NOs: 8, 14, 15, 8, 14, 15, 8, 9, 10, 8, 9, 10, 8, 9, 10, 8, 9, and 10 each, respectively, in order of appearance.

FIGS. 18A and 18B provide plots showing linear modeling of ADT counts identifies the multiplex single-nucleotide polymorphisms (SNP) and rs61939660 as correlates of CD25 expression. FIG. 18A provides a plot showing linear regression modeling was performed to assess which mutated nucleotides correlated to CLR normalized CD25 ADT expression accounting for plate. FIG. 18B provides a plot showing conditioning on dosage at SNP3, linear regression was performed again accounting for plate. Nominal p-values are plotted with the dashed line representing the Bonferroni corrected p-value of 0.05. SNP identities are defined in FIG. 4C.

FIGS. 19A and 19B together provide a schematic of a modified version of MINECRAFTseq. FIG. 19A provides a schematic overview of cell preparation and sorting prior to library preparation. FIG. 19A discloses SEQ ID NOs: 3, 4, 4, 4, and 4, respectively, in order of appearance. FIG. 19B provides a schematic overview of library generation.

### DETAILED DESCRIPTION OF THE INVENTION

The invention features compositions and methods that are useful for characterizing an alteration in a polynucleotide relative to a reference sequence.

The invention is based, at least in part, on the discovery of a technique that provides for the investigation of alterations in a polynucleotide sequence, including alterations associated with CRISPR editing. It can be applied to a wide variety of cells, including cell lines and primary cells. The technique uses flow-assisted sorting of single cells into plates to capture DNA amplicons, total 3′ mRNA, and antibody derived tags (ADT) from CRISPR-edited cells in order to correlate genomic editing in the targeted region with outcomes in protein expression and mRNA. This novel approach takes advantage of a 3′mRNA capture approach with extensive multiplexing to allow for the robust and relatively cheap analysis of tens or even hundreds of thousands of cells. It provides for the simultaneous analysis of genomic editing of DNA, alterations in RNA expression, including characterizing broad expressional changes in genes of interest, and uses Antibody Derived Tags (ADT) to characterize the phenotype of particular cells of interest.

While genetic studies have identified thousands of individual disease driving coding and non-coding alleles, defining the function of these alleles has proven to be a critical bottleneck. CRISPR-Cas gene editing technology has enabled targeted modification of DNA. However, heterogeneous outcomes in successful DNA modifications limit these approaches. In embodiments, invention of the disclosure provides a scalable plate-based single cell approach that simultaneously captures genomic DNA amplicons, mRNA transcriptome, and ADT expression. As described further in the Examples provided herein, this novel multi-omic was used in combination with a breadth of genomic editing techniques, to investigate coding and regulatory alleles in HLADQB1, IL2RA, PTPRC, and UBASH3A in cell lines and primary human CD4 T cells. It is shown in the examples that the combination of single cell editing led to well-powered detection of functional outcomes.

An effective way to rapidly assess the effects of genomic editing is to capture single cell targeted DNA information alongside mRNA and cell surface expression readouts. This approach, as provided in embodiments of the invention of the disclosure, has the advantage of enabling analysis of primary cells, and enables high-powered comparisons of edited and non-edited cells in the same experiment. In embodiments, the methods provided herein are suitable for analysis of primary immune cells or CRISPR edited samples.

### Limitations on Current Approaches

Current approaches for isolating and sequencing single cell DNA and mRNA include Simultaneous Isolation of genomic DNA and total RNA (SIDR), “G&T”, “DR-seq”, and “TARGET-seq”. All of these approaches capture either DNA amplicons or the whole genome along with mRNA using a variety of techniques. Still, these approaches are limited in their scalability, reliability, and efficiency. Additionally, none of these techniques have incorporated antibody derived tags into their protocols and importantly none of them have been applied to CRISPR editing. Other single cell approaches that do focus on CRISPR-edited cells rely entirely on sgRNA expression as a proxy for DNA editing. This is a major limitation to deciphering the varied and heterogeneous effects of editing on mRNA expression. Additionally, these applications rely on integration of DNA into the genome, making them unstable, and can usually only be performed in cell lines, not primary cells.

Multiomic Investigation of Nucleotide Editing by CRISPR with ADT, Flow Cytometry and Transcriptome sequencing (MINECRAFTseq) resolves all of these issues by capturing up to four modalities A) Flow Index Information B) mRNA C) DNA amplicons and D) ADTs from cell lines and primary cells edited with CRISPR reagents. Such methods are useful, for example, for VDJ sequencing for TCR clonotypes (in addition to the other modalities), telomere sequencing to understand relationships relating to cellular age and immunity, to characterize splice isoforms to accurately measure the effects of autoimmune variants on differential isoform usage, and to characterize cancer heterogeneity.

### Single Cell Analysis

MINECRAFTseq provides for the multiomic analysis of single cells. Single cells can be separated using microfluidic devices. Microfluidics involves micro-scale devices that handle small volumes of fluids. Because microfluidics may accurately and reproducibly control and dispense small fluid volumes, in particular volumes less than 1 μl, application of microfluidics provides significant cost-savings. The use of microfluidics technology reduces cycle times, shortens time-to-results, and increases throughput. The small volume of microfluidics technology improves amplification and construction of DNA libraries made from single cells and single isolated aggregations of cellular constituents. Furthermore, incorporation of microfluidics technology enhances system integration and automation.

Single cells of the present invention may be divided into single droplets using a microfluidic device. The single cells in such droplets may be further labeled with a barcode. In this regard reference is made to Macosko et al., 2015, “Highly Parallel Genome-wide Expression Profiling of Individual Cells Using Nanoliter Droplets” Cell 161, 1202-1214 and Klein et al., 2015, “Droplet Barcoding for Single-Cell Transcriptomics Applied to Embryonic Stem Cells” Cell 161, 1187-1201, all the contents and disclosure of each of which are herein incorporated by reference in their entirety.

Microfluidic reactions are generally conducted in microdroplets. The ability to conduct reactions in microdroplets depends on being able to merge different sample fluids and different microdroplets. See, e.g., US Patent Publication No. 20120219947 and PCT publication No. WO2014085802 A1.

Droplet microfluidics (e.g., 10×, DROPSEQ, InDrop) offers significant advantages for performing high-throughput screens and sensitive assays. Droplets allow sample volumes to be significantly reduced, leading to concomitant reductions in cost. Manipulation and measurement at kilohertz speeds enable up to 10⁸ samples to be screened in a single day. Compartmentalization in droplets increases assay sensitivity by increasing the effective concentration of rare species and decreasing the time required to reach detection thresholds. Droplet microfluidics combines these powerful features to enable currently inaccessible high-throughput screening applications, including single-cell and single-molecule assays. See, e.g., Guo et al., Lab Chip, 2012, 12, 2146-2155.

The manipulation of fluids to form fluid streams of desired configuration, discontinuous fluid streams, droplets, particles, dispersions, etc., for purposes of fluid delivery, product manufacture, analysis, and the like, is a relatively well-studied art. Microfluidic systems have been described in a variety of contexts, typically in the context of miniaturized laboratory (e.g., clinical) analysis. Other uses have been described as well. For example, WO 2001/89788; WO 2006/040551; U.S. Patent Application Publication No. 2009/0005254; WO 2006/040554; U.S. Patent Application Publication No. 2007/0184489; WO 2004/002627; U.S. Pat. No. 7,708,949; WO 2008/063227; U.S. Patent Application Publication No. 2008/0003142; WO 2004/091763; U.S. Patent Application Publication No. 2006/0163385; WO 2005/021151; U.S. Patent Application Publication No. 2007/0003442; WO 2006/096571; U.S. Patent Application Publication No. 2009/0131543; WO 2007/089541; U.S. Patent Application Publication No. 2007/0195127; WO 2007/081385; U.S. Patent Application Publication No. 2010/0137163; WO 2007/133710; U.S. Patent Application Publication No. 2008/0014589; U.S. Patent Application Publication No. 2014/0256595; and WO 2011/079176. In a preferred embodiment single molecule analysis is performed in droplets using methods according to WO 2014085802. Each of these patents and publications is herein incorporated by reference in their entireties for all purposes.

### Isolation and Lysis of Single Cells

This disclosure includes the step of isolation of individual cells from a sample, wherein the cells are separated and isolated into individual compartments. The methods used to separate cells will depend, in part, on the origin and type of sample being used. For example separation of individual cells from blood or single cell suspension of tissue can be performed by methods routinely performed in the art, such as flow cytometry or microfluidic techniques (e.g., single-cell sorting using fluorescence-activated cell sorting (FACS) techniques).

In certain embodiments, single cells obtained or separated from tissue are isolated into individual compartments, for example, by placement into individual wells of a tissue culture plate or in microfluidic droplets. In certain embodiments, the individual cells are encapsulated in individual gel beads. In certain aspects, the beads are plastic, glass, silica or metallic and the target biomolecules are released from the beads by a chemical or enzymatic reaction.

In certain embodiments, individual cells are encapsulated in individual oil droplets. In some embodiments, the oil droplets are aqueous solutions surrounded by oil. In certain embodiments, the oil is immiscible with water. In certain embodiments, the oil is transparent. In certain embodiments, the oil droplet has a volume of 1 pL to 100 nL. In certain embodiments, an aqueous solution surrounded by oil comprises buffer solutions. In certain embodiments, a surfactant is added to the oil droplets.

The methods comprise lysis of individual cells to expose target biomolecules for detection. The protocol for lysis of cells depends, in part, upon the nature and sub-cellular location of the target biomolecules to be detected. Any method known in the art for the lysis of membranes and/or extraction of target biomolecules from cells may be employed. Examples of lysis agents include, but are not limited to detergents (e.g., NP-40 (nonyl phenoxypolyethoxylethanol)), surfactants (e.g., non-ionic surfactant such as TritonX-100 and Tween 20, or ionic surfactants such as sarcosyl and sodium dodecyl sulfate), or lysis enzymes (e.g. lysozyme). In certain embodiments, the lysis agents disrupt cellular membranes but do not disrupt oil droplets. In other embodiments, non-reagent based lysis systems can be used including, but not limited to, heat, electroporation, mechanical disruption, and acoustic disruption (e.g., sonication). In an embodiment, the cells are lysed with a solution comprising at least one detergent, surfactant, or lysis enzyme. In certain embodiments, the cells are lysed using a combination of lysis reagents and techniques. In certain embodiments, the surfactant is Triton X-100. In another embodiment, the detergent is NP-40 (nonyl phenoxypolyethoxylethanol). In an embodiment, the cells are lysed with a buffer comprising sodium dodecyl sulfate. In certain embodiments, the cellular material released from the lysed cells comprises cellular proteins. In certain aspects, the lysis of cells is performed in individual single cell compartments.

In certain embodiments, the RNA, DNA and proteins from cells can be separately extracted from individual cells enabling multiplexed transcriptomic, genomic, and/or proteomic analysis from each cell. In an aspect, the RNA, DNA and proteins can be extracted using an extraction reagent that allows for simultaneous isolation of RNA, DNA and protein.

Cell Labeling with Detectable Markers

Cells described herein may be labelled for sorting, and for identification. A detectable marker can be any molecule capable of producing a signal for detecting a target biomolecule. For example, the cell identification detectable marker can be a fluorescent marker. The cell identification detectable marker can comprise, but is not limited to, a fluorescent molecule, chemiluminescent molecule, chromophore, enzyme, enzyme substrate, enzyme cofactor, enzyme inhibitor, dye, metal ion, metal sol, ligand (e.g., biotin, avidin, streptavidin or haptens), radioactive isotope, molecules designed for electronic/ionic detection (e.g., by ISFETs) and the like, and combinations thereof.

Detectable markers can be attached chemically and/or covalently to any appropriate region of the cell identifier probe. In some embodiments, the detectable markers are fluorescent molecules. Fluorescent molecules can be fluorescent proteins or can be a reactive derivative of a fluorescent molecule known as a fluorophore. Fluorophores are fluorescent chemical compounds that emit light upon light excitation. In some embodiments, the fluorophore selectively binds to a specific region or functional group on the target molecule and can be attached chemically or biologically.

Examples of a label which may be employed include labels known to those skilled in the art, such as fluorescent dyes, enzymes, coenzymes, chemiluminescent substances, and radioactive substances as long as the label detects a double-stranded nucleic acid. Specific examples include radioisotopes (e.g., ³²P, ¹⁴C, ¹²⁵I, ³H, and ¹³¹I), fluorescein, rhodamine, dansyl chloride, umbelliferone, luciferase, peroxidase, alkaline phosphatase, β-galactosidase, β-glucosidase, horseradish peroxidase, glucoamylase, lysozyme, saccharide oxidase, microperoxidase, biotin, and ruthenium. In the case where biotin is employed as a labeling substance, preferably, after addition of a biotin-labeled antibody, streptavidin bound to an enzyme (e.g., peroxidase) is further added. Advantageously, the label intercalates within double-stranded DNA, such as ethidium bromide.

Advantageously, the label is a fluorescent label. The dye may be an Evagreen dye or a ROX dye. Examples of fluorescent labels include, but are not limited to, Atto dyes, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′ tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine

The fluorescent label may be a fluorescent protein, such as blue fluorescent protein, cyan fluorescent protein, green fluorescent protein, red fluorescent protein, yellow fluorescent protein or any photoconvertible protein. Colormetric labeling, bioluminescent labeling and/or chemiluminescent labeling may further accomplish labeling. Labeling further may include energy transfer between molecules in the hybridization complex by perturbation analysis, quenching, or electron transport between donor and acceptor molecules, the latter of which may be facilitated by double stranded match hybridization complexes. The fluorescent label may be a perylene or a terrylen. In the alternative, the fluorescent label may be a fluorescent bar code.

The label may be a fluorescent label, advantageously fluorescein or rhodamine. In another embodiment, the label may be an organic label. In some embodiments, fluorescent tags useful in the methods of this disclosre include, but are not limited to, green fluorescent protein (GFP), yellow fluorescent protein (YFP), red fluorescent protein (RFP), cyan fluorescent protein (CFP), fluorescein, fluorescein isothiocyanate (FITC), tetramethylrhodamine isothiocyanate (TRITC), cyanine (Cy3), phycoerythrin (R-PE) 5,6-carboxymethyl fluorescein, (5-carboxyfluorescein-N-hydroxysuccinimide ester), Texas red, nitrobenz-2-oxa-1,3-diazol-4-yl (NBD), coumarin, dansyl chloride, and rhodamine (5,6-tetramethyl rhodamine).

In certain embodiments the detection markers are configured for electronic detection. For example, the detectable marker can release ions upon a subsequent reaction, changing the pH of its environment in a manner that is reliably detectable.

### Barcoding of Polynucleotides

In the context of a polynucleotide, a barcode refers to any unique, non-naturally occurring, nucleic acid sequence that may be used to identify the originating source of a nucleic acid fragment. Such barcodes may be sequences including but not limited to, TTGAGCCT, AGTTGCTT, CCAGTTAG, ACCAACTG, GTATAACA or CAGGAGCC. Although it is not necessary to understand the mechanism of an invention, it is believed that the barcode sequence provides a high-quality individual read of a barcode associated with a particular polynucleotide (e.g., labeling ligand, shRNA, sgRNA or cDNA) such that multiple species can be sequenced together. Further, these putative barcode loci are believed short enough to be easily sequenced with current technology. Kress et al., “DNA barcodes: Genes, genomics, and bioinformatics” PNAS 105(8):2761-2762 (2008).

Software for DNA barcoding requires integration of a field information management system (FIMS), laboratory information management system (LIMS), sequence analysis tools, workflow tracking to connect field data and laboratory data, database submission tools and pipeline automation for scaling up to eco-system scale projects. Geneious Pro can be used for the sequence analysis components, and the two plugins made freely available through the Moorea Biocode Project, the Biocode LIMS and Genbank Submission plugins handle integration with the FIMS, the LIMS, workflow tracking and database submission.

Additionally, other barcoding designs and tools have been described (see e.g., Birrell et al., (2001) Proc. Natl Acad. Sci. USA 98, 12608-12613; Giaever, et al., (2002) Nature 418, 387-391; Winzeler et al., (1999) Science 285, 901-906; and Xu et al., (2009) Proc Natl Acad Sci USA. February 17; 106(7):2289-94).

Cell identifier oligonucleotide barcodes may be any length that allows efficient binding to a target sequence. In certain aspects, the cell identifier oligonucleotide barcodes are less than 200 nucleotides in length, less than 100 nucleotides in length, less than 80 nucleotides in length, less than 50 nucleotides in length, less than 40 nucleotides in length, less than 30 nucleotides in length or less than 20 nucleotides in length. The complementarity of the cell identifier oligonucleotide barcodes to the cell identifier probe oligonucleotide is a precise pairing such that stable and specific binding occurs between nucleic acid sequences e.g., between a cell identifier probe oligonucleotide sequence and the cell identifier oligonucleotide barcode sequence (e.g., nucleotide sequence variant) of interest. It is understood that the sequence of a nucleic acid need not be 100% complementary to that of its target or complement. In some cases, the sequence is complementary to the other sequence with the exception of 1-2 mismatches. In some cases, the sequences are complementary except for 1 mismatch. In some cases, the sequences are complementary except for 2 mismatches. In some cases, the sequences are complementary except for 3 mismatches. In yet other cases, the sequences are complementary except for 4, 5, 6, 7, 8, 9 or more mismatches. In certain aspects, the number of mismatches is 20% or less, 10% or less, 5% or less or 2% or less of the number of nucleotides present in the cell identifier oligonucleotide barcode. In certain aspects, the cell identifier oligonucleotide barcode and the cell identifier probe oligonucleotide are complementary to at least 18, at least 17, at least 16, at least 15, at least 14, at least 13, at least 12, at least 11, at least 1, at least 9, at least 8, at least 7, at least 6 or at least 5 nucleotides of a target nucleotide sequence. In certain aspects, tags are complementary to one or more individual probes. In certain aspects, the tags do not bind to alternative sequences because of mismatches in sequences leading to loss of complementarity.

In certain embodiments, cell identifier tags are conjugated or bound to target biomolecules using enzymatic conjugation.

Methods for the synthesis of barcodes include, in certain embodiments, random addition of mixed bases during nucleic acid synthesis to produce a sequence that can be used to identify a specific oligonucleotide molecule through analysis of sequencing data. In certain embodiments, synthesis of barcodes comprises the controlled addition of bases to generate a known sequence. In certain embodiments, barcode sequences can be verified by sequencing. In certain aspects, barcodes can be synthesized and extended using polymerase to attach the barcode to oligonucleotides on probes and tags such as, cell identifier probes, target detection probes, cell identifier tags and target identification tags. In other aspects, barcode sequences can be synthesized without probes and either ligated or annealed to the probes in a separate step.

### Oligonucleotide Conjugates

In certain embodiments, an assay described herein comprises contacting cellular material from single cells (e.g., DNA, RNA) with oligonucleotides conjugated with an antibody. Oligonucleotides can be conjugated to antibodies by a number of methods known in the art (Kozlov et al., “Efficient strategies for the conjugation of oligonucleotides to antibodies enabling highly sensitive protein detection”; Biopolymers; 73(5); Apr. 5, 2004; pp. 621-630). Aldehydes can be introduced to antibodies by modification of primary amines or oxidation of carbohydrate residues. Aldehyde- or hydrazine-modified oligonucleotides are prepared either during phosphoramidite synthesis or by post-synthesis derivatization. Conjugation between the modified oligonucleotide and antibody result in the formation of a hydrazone bond that is stable over long periods of time under physiological conditions. Oligonucleotides can also be conjugated to antibodies by producing chemical handles through thiol/maleimide chemistry, azide/alkyne chemistry, tetrazine/cyclooctyne chemistry and other click chemistries. These chemical handles are prepared either during phosphoramidite synthesis or post-synthesis.

In one embodiment, the oligonucleotide-antibody conjugates are designed for use with single-cell sequencing platforms that rely on Poly-dT oligonucleotides as the mRNA capture method (scRNA-seq). The antibodies integrate in the scRNA-seq workflow by mimicking natural mRNA, thanks to the poly-A tail sequence in the conjugated oligonucleotide. The oligonucleotide also contains a barcode that permanently labels a specific clone, and a PCR handle, which makes it compatible with Illumina® sequencing reagents and instruments.

### Cell Hashing

In some embodiments, oligonucleotide-tagged antibodies are used to convert the detection of cell surface proteins into a sequenceable readout alongside scRNA-seq. A defined set of oligo-tagged antibodies against ubiquitous surface proteins is used to uniquely label different experimental samples. This enables these samples to be pooled together. The barcoded antibody signal is used as a fingerprint for reliable demultiplexing. This approach is referred to as Cell Hashing, based on the concept of hash functions in computer science to index datasets with specific features; our set of oligo-derived hashtags equally define a “lookup table” to assign each multiplexed cell to its original sample.

In embodiments, Cell Hashing involves the use of oligo-tagged antibodies against ubiquitously expressed surface proteins uniquely label cells from distinct samples, which can be subsequently pooled. By sequencing these tags alongside the cellular transcriptome, each cell is assigned to its original sample, robustly identify cross-sample multiplets, and “super-load” commercial droplet-based systems for significant cost reduction. Hashing can generalize the benefits of single cell multiplexing to diverse samples and experimental designs.

### Sequencing

The gDNA, cDNA, and ADT libraries described herein are amenable to virtually any sequencing method known in the art. In some embodiments, sequencing is carried out using next generation sequencing technology (NGS), which allows massively parallel sequencing. In certain embodiments, clonally amplified DNA templates or single DNA molecules are sequenced in a massively parallel fashion within a flow cell (e.g., as described in Volkerding et al. Clin Chem 55:641-658 [2009]; Metzker M Nature Rev 11:31-46 [2010]). The sequencing technologies of NGS include but are not limited to pyrosequencing, sequencing-by-synthesis with reversible dye terminators, sequencing by oligonucleotide probe ligation, and ion semiconductor sequencing. DNA from individual samples can be sequenced individually (i.e., singleplex sequencing) or DNA from multiple samples can be pooled and sequenced as indexed genomic molecules (i.e., multiplex sequencing) on a single sequencing run, to generate up to several hundred million reads of DNA sequences. Examples of sequencing technologies that can be used to obtain the sequence information according to the present method are further described here.

Some sequencing technologies are available commercially, such as the sequencing-by-hybridization platform from Affymetrix Inc. (Sunnyvale, Calif.) and the sequencing-by-synthesis platforms from 454 Life Sciences (Bradford, Conn.), Illumina/Solexa (Hayward, Calif.) and Helicos Biosciences (Cambridge, Mass.), and the sequencing-by-ligation platform from Applied Biosystems (Foster City, Calif), as described below. In addition to the single molecule sequencing performed using sequencing-by-synthesis of Helicos Biosciences, other single molecule sequencing technologies include, but are not limited to, the SMRT™ technology of Pacific Biosciences, the ION TORRENT′ technology, and nanopore sequencing developed for example, by Oxford Nanopore Technologies.

While the automated Sanger method is considered as a ‘first generation’ technology, Sanger sequencing including the automated Sanger sequencing, can also be employed in the methods described herein. Additional suitable sequencing methods include, but are not limited to nucleic acid imaging technologies, e.g., atomic force microscopy (AFM) or transmission electron microscopy (TEM). Illustrative sequencing technologies are described in greater detail below.

In some embodiments, methods provided herein involve obtaining sequence information for the nucleic acids in a test sample by massively parallel sequencing of millions of DNA fragments using Illumina's sequencing-by-synthesis and reversible terminator-based sequencing chemistry (e.g. as described in Bentley et al., Nature 6:53-59 [2009]). Template DNA can be genomic DNA, e.g., cellular DNA or cDNA. In some embodiments, genomic DNA from isolated cells is used as the template, and it is fragmented into lengths of several hundred base pairs. In some embodiments, Illumina's sequencing technology relies on the attachment of fragmented genomic DNA to a planar, optically transparent surface on which oligonucleotide anchors are bound. Template DNA is end-repaired to generate 5′-phosphorylated blunt ends, and the polymerase activity of Klenow fragment is used to add a single A base to the 3′ end of the blunt phosphorylated DNA fragments. This addition prepares the DNA fragments for ligation to oligonucleotide adapters, which have an overhang of a single T base at their 3′ end to increase ligation efficiency. The adapter oligonucleotides are complementary to the flow-cell anchor oligos. Under limiting-dilution conditions, adapter-modified, single-stranded template DNA is added to the flow cell and immobilized by hybridization to the anchor oligos. Attached DNA fragments are extended and bridge amplified to create an ultra-high density sequencing flow cell with hundreds of millions of clusters, each containing about 1,000 copies of the same template.

In one embodiment, the randomly fragmented library DNA (e.g., genomic DNA, cDNA) is amplified using PCR before it is subjected to cluster amplification. Alternatively or in addition, an amplification-free genomic library preparation is used, and the randomly fragmented genomic DNA or other polynucleotide is enriched using the cluster amplification alone (Kozarewa et al., Nature Methods 6:291-295 [2009]). In some applications, the templates are sequenced using a robust four-color DNA sequencing-by-synthesis technology that employs reversible terminators with removable fluorescent dyes. High-sensitivity fluorescence detection is achieved using laser excitation and total internal reflection optics. Short sequence reads of about tens to a few hundred base pairs are aligned against a reference genome and unique mapping of the short sequence reads to the reference genome are identified using specially developed data analysis pipeline software. After completion of the first read, the templates can be regenerated in situ to enable a second read from the opposite end of the fragments. Thus, either single-end or paired end sequencing of the DNA fragments can be used.

Various embodiments of the disclosure may use sequencing by synthesis that allows paired end sequencing. In some embodiments, the sequencing by synthesis platform by Illumina involves clustering fragments. Clustering is a process in which each fragment molecule is isothermally amplified. In some embodiments, as the example described here, the fragment has two different adapters attached to the two ends of the fragment, the adapters allowing the fragment to hybridize with the two different oligos on the surface of a flow cell lane. The fragment further includes or is connected to two index sequences at two ends of the fragment, which index sequences provide labels to identify different samples in multiplex sequencing. In some sequencing platforms, a fragment to be sequenced from both ends is also referred to as an insert.

In some implementation, a flow cell for clustering in the Illumina platform is a glass slide with lanes. Each lane is a glass channel coated with a lawn of two types of oligos (e.g., P5 and P7′ oligos). Hybridization is enabled by the first of the two types of oligos on the surface. This oligo is complementary to a first adapter on one end of the fragment. A polymerase creates a compliment strand of the hybridized fragment. The double-stranded molecule is denatured, and the original template strand is washed away. The remaining strand, in parallel with many other remaining strands, is clonally amplified through bridge application.

In bridge amplification and other sequencing methods involving clustering, a strand folds over, and a second adapter region on a second end of the strand hybridizes with the second type of oligos on the flow cell surface. A polymerase generates a complementary strand, forming a double-stranded bridge molecule. This double-stranded molecule is denatured resulting in two single-stranded molecules tethered to the flow cell through two different oligos. The process is then repeated over and over, and occurs simultaneously for millions of clusters resulting in clonal amplification of all the fragments. After bridge amplification, the reverse strands are cleaved and washed off, leaving only the forward strands. The 3′ ends are blocked to prevent unwanted priming.

After clustering, sequencing starts with extending a first sequencing primer to generate the first read. With each cycle, fluorescently tagged nucleotides compete for addition to the growing chain. Only one is incorporated based on the sequence of the template. After the addition of each nucleotide, the cluster is excited by a light source, and a characteristic fluorescent signal is emitted. The number of cycles determines the length of the read. The emission wavelength and the signal intensity determine the base call. For a given cluster all identical strands are read simultaneously. Hundreds of millions of clusters are sequenced in a massively parallel manner. At the completion of the first read, the read product is washed away.

In the next step of protocols involving two index primers, an index 1 primer is introduced and hybridized to an index 1 region on the template. Index regions provide identification of fragments, which is useful for de-multiplexing samples in a multiplex sequencing process. The index 1 read is generated similar to the first read. After completion of the index 1 read, the read product is washed away and the 3′ end of the strand is de-protected. The template strand then folds over and binds to a second oligo on the flow cell. An index 2 sequence is read in the same manner as index 1. Then an index 2 read product is washed off at the completion of the step.

After reading two indices, read 2 initiates by using polymerases to extend the second flow cell oligos, forming a double-stranded bridge. This double-stranded DNA is denatured, and the 3′ end is blocked. The original forward strand is cleaved off and washed away, leaving the reverse strand. Read 2 begins with the introduction of a read 2 sequencing primer. As with read 1, the sequencing steps are repeated until the desired length is achieved. The read 2 product is washed away. This entire process generates millions of reads, representing all the fragments. Sequences from pooled sample libraries are separated based on the unique indices introduced during sample preparation. For each sample, reads of similar stretches of base calls are locally clustered. Forward and reversed reads are paired creating contiguous sequences. These contiguous sequences are aligned to the reference genome for variant identification.

Sequencing by synthesis involves paired end reads. Paired end sequencing involves 2 reads from the two ends of a fragment. Paired end reads are used to resolve ambiguous alignments. Paired-end sequencing allows users to choose the length of the insert (or the fragment to be sequenced) and sequence either end of the insert, generating high-quality, alignable sequence data. Because the distance between each paired read is known, alignment algorithms can use this information to map reads over repetitive regions more precisely. This results in better alignment of the reads, especially across difficult-to-sequence, repetitive regions of the genome. Paired-end sequencing can detect rearrangements, including insertions and deletions (indels) and inversions.

Paired end reads may use insert of different length (i.e., different fragment size to be sequenced). As the default meaning in this disclosure, paired end reads are used to refer to reads obtained from various insert lengths. In some instances, to distinguish short-insert paired end reads from long-inserts paired end reads, the latter is specifically referred to as mate pair reads. In some embodiments involving mate pair reads, two biotin junction adapters first are attached to two ends of a relatively long insert (e.g., several kb). The biotin junction adapters then link the two ends of the insert to form a circularized molecule. A sub-fragment encompassing the biotin junction adapters can then be obtained by further fragmenting the circularized molecule. The sub-fragment including the two ends of the original fragment in opposite sequence order can then be sequenced by the same procedure as for short-insert paired end sequencing described above. Further details of mate pair sequencing using an Illumina platform is shown in an online publication at the following address, which is incorporated by reference by its entirety: res.illumina.com/documents/products/technotes/technote_nextera_matepair_data_processing.pdf

After sequencing of DNA fragments, sequence reads of predetermined length, e.g., 100 bp, are localized by mapping (alignment) to a known reference genome. The mapped reads and their corresponding locations on the reference sequence are also referred to as tags. In another embodiment of the procedure, localization is realized by k-mer sharing and read-read alignment. The analyses of many embodiments disclosed herein make use of reads that are either poorly aligned or cannot be aligned, as well as aligned reads (tags). In one embodiment, the reference genome sequence is the NCBI36/hg18 sequence, which is available on the World Wide Web at genome.ucsc.edu/cgi-bin/hgGateway?org=Human&db=hg18&hgsid=166260105). Alternatively, the reference genome sequence is the GRCh37/hg19 or GRCh38, which is available on the World Wide Web at genome.ucsc.edu/cgi-bin/hgGateway. Other sources of public sequence information include GenBank, dbEST, dbSTS, EMBL (the European Molecular Biology Laboratory), and the DDBJ (the DNA Databank of Japan). A number of computer algorithms are available for aligning sequences, including without limitation BLAST (Altschul et al., 1990), BLITZ (MPsrch) (Sturrock & Collins, 1993), FASTA (Person & Lipman, 1988), BOWTIE (Langmead et al., Genome Biology 10:R25.1-R25.10 [2009]), or ELAND (Illumina, Inc., San Diego, Calif, USA). In one embodiment, one end of the clonally expanded copies of the plasma cfDNA molecules is sequenced and processed by bioinformatics alignment analysis for the Illumina Genome Analyzer, which uses the Efficient Large-Scale Alignment of Nucleotide Databases (ELAND) software.

In one illustrative, but non-limiting, embodiment, the methods described herein include obtaining sequence information for the nucleic acids in a test sample, using single molecule sequencing technology of the Helicos True Single Molecule Sequencing (tSMS) technology (e.g. as described in Harris T. D. et al., Science 320:106-109 [2008]). In the tSMS technique, a DNA sample is cleaved into strands of approximately 100 to 200 nucleotides, and a polyA sequence is added to the 3′ end of each DNA strand. Each strand is labeled by the addition of a fluorescently labeled adenosine nucleotide. The DNA strands are then hybridized to a flow cell, which contains millions of oligo-T capture sites that are immobilized to the flow cell surface. In certain embodiments the templates can be at a density of about 100 million templates/cm². The flow cell is then loaded into an instrument, e.g., HeliScope™ sequencer, and a laser illuminates the surface of the flow cell, revealing the position of each template. A CCD camera can map the position of the templates on the flow cell surface. The template fluorescent label is then cleaved and washed away. The sequencing reaction begins by introducing a DNA polymerase and a fluorescently labeled nucleotide. The oligo-T nucleic acid serves as a primer. The polymerase incorporates the labeled nucleotides to the primer in a template directed manner. The polymerase and unincorporated nucleotides are removed. The templates that have directed incorporation of the fluorescently labeled nucleotide are discerned by imaging the flow cell surface. After imaging, a cleavage step removes the fluorescent label, and the process is repeated with other fluorescently labeled nucleotides until the desired read length is achieved. Sequence information is collected with each nucleotide addition step. Whole genome sequencing by single molecule sequencing technologies excludes or typically obviates PCR-based amplification in the preparation of the sequencing libraries, and the methods allow for direct measurement of the sample, rather than measurement of copies of that sample.

In another illustrative, but non-limiting embodiment, the methods described herein include obtaining sequence information for the nucleic acids in the test sample, using the 454 sequencing (Roche) (e.g. as described in Margulies, M. et al. Nature 437:376-380 [2005]). 454 sequencing typically involves two steps. In the first step, DNA is sheared into fragments of approximately 300-800 base pairs, and the fragments are blunt-ended. Oligonucleotide adapters are then ligated to the ends of the fragments. The adapters serve as primers for amplification and sequencing of the fragments. The fragments can be attached to DNA capture beads, e.g., streptavidin-coated beads using, e.g., adapter B, which contains 5′-biotin tag. The fragments attached to the beads are PCR amplified within droplets of an oil-water emulsion. The result is multiple copies of clonally amplified DNA fragments on each bead. In the second step, the beads are captured in wells (e.g., picoliter-sized wells). Pyrosequencing is performed on each DNA fragment in parallel. Addition of one or more nucleotides generates a light signal that is recorded by a CCD camera in a sequencing instrument. The signal strength is proportional to the number of nucleotides incorporated. Pyrosequencing makes use of pyrophosphate (PPi) which is released upon nucleotide addition. PPi is converted to ATP by ATP sulfurylase in the presence of adenosine 5′ phosphosulfate. Luciferase uses ATP to convert luciferin to oxyluciferin, and this reaction generates light that is measured and analyzed.

In another illustrative, but non-limiting, embodiment, the methods described herein includes obtaining sequence information for the nucleic acids in the test sample, using the SOLiD™ technology (Applied Biosystems). In SOLiD™ sequencing-by-ligation, genomic DNA is sheared into fragments, and adapters are attached to the 5′ and 3′ ends of the fragments to generate a fragment library. Alternatively, internal adapters can be introduced by ligating adapters to the 5′ and 3′ ends of the fragments, circularizing the fragments, digesting the circularized fragment to generate an internal adapter, and attaching adapters to the 5′ and 3′ ends of the resulting fragments to generate a mate-paired library. Next, clonal bead populations are prepared in microreactors containing beads, primers, template, and PCR components. Following PCR, the templates are denatured and beads are enriched to separate the beads with extended templates. Templates on the selected beads are subjected to a 3′ modification that permits bonding to a glass slide. The sequence can be determined by sequential hybridization and ligation of partially random oligonucleotides with a central determined base (or pair of bases) that is identified by a specific fluorophore. After a color is recorded, the ligated oligonucleotide is cleaved and removed and the process is then repeated.

In another illustrative, but non-limiting, embodiment, the methods described herein include obtaining sequence information for the nucleic acids in the test sample, using the single molecule, real-time (SMRT™) sequencing technology of Pacific Biosciences. In SMRT sequencing, the continuous incorporation of dye-labeled nucleotides is imaged during DNA synthesis. Single DNA polymerase molecules are attached to the bottom surface of individual zero-mode wavelength detectors (ZMW detectors) that obtain sequence information while phospholinked nucleotides are being incorporated into the growing primer strand. A ZMW detector includes a confinement structure that enables observation of incorporation of a single nucleotide by DNA polymerase against a background of fluorescent nucleotides that rapidly diffuse in an out of the ZMW (e.g., in microseconds). It typically takes several milliseconds to incorporate a nucleotide into a growing strand. During this time, the fluorescent label is excited and produces a fluorescent signal, and the fluorescent tag is cleaved off. Measurement of the corresponding fluorescence of the dye indicates which base was incorporated. The process is repeated to provide a sequence.

In another illustrative, but non-limiting embodiment, the methods described herein include obtaining sequence information for the nucleic acids in the test sample, using nanopore sequencing (e.g. as described in Soni G V and Meller A. Clin Chem 53: 1996-2001 [2007]). Nanopore sequencing DNA analysis techniques are developed by a number of companies, including, for example, Oxford Nanopore Technologies (Oxford, United Kingdom), Sequenom, NABsys, and the like. Nanopore sequencing is a single-molecule sequencing technology whereby a single molecule of DNA is sequenced directly as it passes through a nanopore. A nanopore is a small hole, typically of the order of 1 nanometer in diameter. Immersion of a nanopore in a conducting fluid and application of a potential (voltage) across it results in a slight electrical current due to conduction of ions through the nanopore. The amount of current that flows is sensitive to the size and shape of the nanopore. As a DNA molecule passes through a nanopore, each nucleotide on the DNA molecule obstructs the nanopore to a different degree, changing the magnitude of the current through the nanopore in different degrees. Thus, this change in the current as the DNA molecule passes through the nanopore provides a read of the DNA sequence.

In another illustrative, but non-limiting, embodiment, the methods described herein includes obtaining sequence information for the nucleic acids in the test sample, using the chemical-sensitive field effect transistor (chemFET) array (e.g., as described in U.S. Patent Application Publication No. 2009/0026082). In one example of this technique, DNA molecules can be placed into reaction chambers, and the template molecules can be hybridized to a sequencing primer bound to a polymerase. Incorporation of one or more triphosphates into a new nucleic acid strand at the 3′ end of the sequencing primer can be discerned as a change in current by a chemFET. An array can have multiple chemFET sensors. In another example, single nucleic acids can be attached to beads, and the nucleic acids can be amplified on the bead, and the individual beads can be transferred to individual reaction chambers on a chemFET array, with each chamber having a chemFET sensor, and the nucleic acids can be sequenced.

Ion Torrent PGM™ sequencer (Life Technologies) and the Ion Torrent Proton™ Sequencer (Life Technologies) are ion-based sequencing systems that sequence nucleic acid templates by detecting ions produced as a byproduct of nucleotide incorporation. Typically, hydrogen ions are released as byproducts of nucleotide incorporations occurring during template-dependent nucleic acid synthesis by a polymerase. The Ion Torrent PGM™ sequencer and Ion Torrent Proton™ Sequencer detect the nucleotide incorporations by detecting the hydrogen ion byproducts of the nucleotide incorporations. The Ion Torrent PGM™ sequencer and Ion Torrent Proton™ sequencer include a plurality of nucleic acid templates to be sequenced, each template disposed within a respective sequencing reaction well in an array. The wells of the array are each coupled to at least one ion sensor that can detect the release of H+ ions or changes in solution pH produced as a byproduct of nucleotide incorporation. The ion sensor comprises a field effect transistor (FET) coupled to an ion-sensitive detection layer that can sense the presence of H+ ions or changes in solution pH. The ion sensor provides output signals indicative of nucleotide incorporation which can be represented as voltage changes whose magnitude correlates with the H+ ion concentration in a respective well or reaction chamber. Different nucleotide types are flowed serially into the reaction chamber, and are incorporated by the polymerase into an extending primer (or polymerization site) in an order determined by the sequence of the template. Each nucleotide incorporation is accompanied by the release of H+ ions in the reaction well, along with a concomitant change in the localized pH. The release of H+ ions is registered by the FET of the sensor, which produces signals indicating the occurrence of the nucleotide incorporation. Nucleotides that are not incorporated during a particular nucleotide flow will not produce signals. The amplitude of the signals from the FET may also be correlated with the number of nucleotides of a particular type incorporated into the extending nucleic acid molecule thereby permitting homopolymer regions to be resolved. Thus, during a run of the sequencer multiple nucleotide flows into the reaction chamber along with incorporation monitoring across a multiplicity of wells or reaction chambers permit the instrument to resolve the sequence of many nucleic acid templates simultaneously. Further details regarding the compositions, design and operation of the Ion Torrent PGM™ sequencer can be found, for example, in U.S. patent application Ser. No. 12/002,781, now published as U.S. Patent Publication No. 2009/0026082; U.S. patent application Ser. No. 12/474,897, now published as U.S. Patent Publication No. 2010/0137143; and U.S. patent application Ser. No. 12/492,844, now published as U.S. Patent Publication No. 2010/0282617, all of which applications are incorporated by reference herein in their entireties. In some embodiments, amplicons can be manipulated or amplified through bridge amplification or emPCR to generate a plurality of clonal templates that are suitable for a variety of downstream processes including nucleic acid sequencing. In one embodiment, nucleic acid templates to be sequenced using the Ion Torrent PGM™ or Ion Proton PGM™ system can be prepared from a population of nucleic acid molecules using one or more of the target-specific amplification techniques outlined herein. Optionally, following target-specific amplification a secondary and/or tertiary amplification process including, but not limited to a library amplification step and/or a clonal amplification step such as emPCR can be performed. The use of such next generation sequencers is contemplated herein for rapidly characterizing at a single cell level alterations in gDNA, cDNA and ADT libraries relative to reference sequence.

In another embodiment, the present method includes obtaining sequence information for the nucleic acids in the test sample, using sequencing by hybridization. Sequencing-by-hybridization involves contacting the plurality of polynucleotide sequences with a plurality of polynucleotide probes, wherein each of the plurality of polynucleotide probes can be optionally tethered to a substrate. The substrate might be flat surface including an array of known nucleotide sequences. The pattern of hybridization to the array can be used to determine the polynucleotide sequences present in the sample. In other embodiments, each probe is tethered to a bead, e.g., a magnetic bead or the like. Hybridization to the beads can be determined and used to identify the plurality of polynucleotide sequences within the sample.

In some embodiments of the methods described herein, the sequence reads are about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. It is expected that technological advances will enable single-end reads of greater than 500 bp enabling for reads of greater than about 1000 bp when paired end reads are generated. In some embodiments, paired end reads are used to determine sequences of interest, which include sequence reads that are about 20 bp to 1000 bp, about 50 bp to 500 bp, or 80 bp to 150 bp. In various embodiments, the paired end reads are used to evaluate a sequence of interest. The sequence of interest is longer than the reads. In some embodiments, the sequence of interest is longer than about 100 bp, 500 bp, 1000 bp, or 4000 bp. Mapping of the sequence reads is achieved by comparing the sequence of the reads with the sequence of the reference to determine the chromosomal origin of the sequenced nucleic acid molecule, and specific genetic sequence information is not needed. A small degree of mismatch (0-2 mismatches per read) may be allowed to account for minor polymorphisms that may exist between the reference genome and the genomes in the mixed sample. In some embodiments, reads that are aligned to the reference sequence are used as anchor reads, and reads paired to anchor reads but cannot align or poorly align to the reference are used as anchored reads. In some embodiments, poorly aligned reads may have a relatively large number of percentage of mismatches per read, e.g., at least about 5%, at least about 10%, at least about 15%, or at least about 20% mismatches per read.

A plurality of sequence tags (i.e., reads aligned to a reference sequence) are typically obtained per sample. In some embodiments, at least about 3×10⁶ sequence tags, at least about 5×10⁶ sequence tags, at least about 8×10⁶ sequence tags, at least about 10×10⁶ sequence tags, at least about 15×10⁶ sequence tags, at least about 20×10⁶ sequence tags, at least about 30×10⁶ sequence tags, at least about 40×10⁶ sequence tags, or at least about 50×10⁶ sequence tags of, e.g., 100 bp, are obtained from mapping the reads to the reference genome per sample. In some embodiments, all the sequence reads are mapped to all regions of the reference genome, providing genome-wide reads. In other embodiments, reads mapped to a sequence of interest.

### Implementation in Hardware

In various aspects, the methods described herein are conducted with the aid of a computer-based system configured to execute machine-readable instructions, which, when executed by a processor of the system causes the system to perform steps including determining the identity, size, nucleotide sequence or other measurable characteristics of the amplicons produced in the method of the invention. One or more features of any one or more of the above-discussed teachings and/or exemplary embodiments may be performed or implemented using appropriately configured and/or programmed hardware and/or software elements. Determining whether an embodiment is implemented using hardware and/or software elements may be based on any number of factors, such as desired computational rate, power levels, heat tolerances, processing cycle budget, input data rates, output data rates, memory resources, data bus speeds, etc., and other design or performance constraints.

Examples of hardware elements may include processors, microprocessors, input(s) and/or output(s) (I/O) device(s) (or peripherals) that are communicatively coupled via a local interface circuit, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. The local interface may include, for example, one or more buses or other wired or wireless connections, controllers, buffers (caches), drivers, repeaters and receivers, etc., to allow appropriate communications between hardware components. A processor is a hardware device for executing software, particularly software stored in memory. The processor can be any custom made or commercially available processor, a central processing unit (CPU), an auxiliary processor among several processors associated with the computer, a semiconductor based microprocessor (e.g., in the form of a microchip or chip set), a macroprocessor, or generally any device for executing software instructions. A processor can also represent a distributed processing architecture. The I/O devices can include input devices, for example, a keyboard, a mouse, a scanner, a microphone, a touch screen, an interface for various medical devices and/or laboratory instruments, a bar code reader, a stylus, a laser reader, a radio-frequency device reader, etc. Furthermore, the I/O devices also can include output devices, for example, a printer, a bar code printer, a display, etc. Finally, the I/O devices further can include devices that communicate as both inputs and outputs, for example, a modulator/demodulator (modem; for accessing another device, system, or network), a radio frequency (RF) or other transceiver, a telephonic interface, a bridge, a router, etc.

Examples of software may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. A software in memory may include one or more separate programs, which may include ordered listings of executable instructions for implementing logical functions. The software in memory may include a system for identifying data streams in accordance with the present teachings and any suitable custom made or commercially available operating system (O/S), which may control the execution of other computer programs such as the system, and provides scheduling, input-output control, file and data management, memory management, communication control, etc.

According to various exemplary embodiments, one or more features of any one or more of the above-discussed teachings and/or exemplary embodiments may be performed or implemented at least partly using a distributed, clustered, remote, or cloud computing resource.

According to various exemplary embodiments, one or more features of any one or more of the above-discussed teachings and/or exemplary embodiments may be performed or implemented using a source program, executable program (object code), script, or any other entity comprising a set of instructions to be performed. When using a source program, the program can be translated via a compiler, assembler, interpreter, etc., which may or may not be included within the memory, so as to operate properly in connection with the O/S. The instructions may be written using (a) an object oriented programming language, which has classes of data and methods, or (b) a procedural programming language, which has routines, subroutines, and/or functions, which may include, for example, C, C++, Pascal, Basic, Fortran, Cobol, Pert, Java, and Ada.

According to various exemplary embodiments, one or more of the above-discussed exemplary embodiments may include transmitting, displaying, storing, printing or outputting to a user interface device, a computer readable storage medium, a local computer system or a remote computer system, information related to any information, signal, data, and/or intermediate or final results that may have been generated, accessed, or used by such exemplary embodiments. Such transmitted, displayed, stored, printed or outputted information can take the form of searchable and/or filterable lists of runs and reports, pictures, tables, charts, graphs, spreadsheets, correlations, sequences, and combinations thereof, for example.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

MINECRAFTseq is a single cell multi-omic approach that captures DNA amplicons, 3′ mRNA transcripts, antibody derived tags (ADT), and index flow sorting information from CRISPR-edited and sorted cells. The method can be applied to cell lines and primary blood cells, particularly B and T cells, to simultaneously examine the effects of CRISPR editing and the outcome on RNA and cell surface expression. It is a highly adaptable technique that can be used on any cell lines and primary edited cells. It is also highly modular and scalable using automated liquid handing.

The technique relies on sorted and pooled cells in order to control for plate and sample effects. The technique can also be applied on low-input <1000 cells for a bulk multi-omic estimate. Importantly, no technique to date that capture DNA and mRNA has been applied to CRISPR edited cells, instead focusing on cancer related heterogeneity and applications.

As described in the Examples below, MINECRAFTseq was used to characterize the following CRISPR edits:
a. CRISPR-Cas9 cutting in cell lines can be used to examine regulatory regions in detail.
b. CRISPR-Cas Base Editing (BE4) can be performed in cell lines to examine variant to function
c. CRISPR-Cas Base Editing can be performed in primary cells to investigate gene knockout
d. CRISPR-Cas Base Editing can be performed in primary cells to examine autoimmune variants
e. CRISPR-Cas Base Editing can be multiplexed in primary cells
f. CRISPR-Cas HDR can be performed in primary cells

The MINECRAFTseq method involves can be started with CRISPR-edited cell lines or primary cells and relies on sorting single cells using a FACS sorter such as an ARIA II (BD) into either 96 or 384 well plates for further processing into sequencing libraries. Processing of plates can be automated using liquid handling platforms to reduce volumes.

Cell lines obtained from ATCC are nucleofected with CRISPR reagents to induce genomic perturbations. The exact conditions for these nucleofections are highly dependent on the cell of choice and reagents used. To date, we have successfully performed genomic editing on HH and Jurkat T cell lines with CRISPR-Cas RNP (Ribonuclear Proteins) with or without a single stranded oligo donor for HDR repair and base-editing Cas mRNAs. For primary immune cells, we have genomically edited primary T cells with the same set of reagents.

After cells are genomically edited with any number of conditions, they are subjected to MINECRAFTseq—a protocol that sorts and prepares single cell libraries for sequencing. The protocol is divided into 6 sections.

Briefly, cells are labeled with antibodies, single-cell index sorted into plates, and lysed in the presence of proteases. Reverse transcription with a template switch oligo is performed to convert mRNA to cDNA and add well-specific barcodes and UMIs. The cDNA along with the ADT and specific genomic DNA is amplified at this stage in one large pool per well. After amplification, a sample of the product is used for further amplification with nested and barcoded primers, adding a well-specific identifier. At this point, the DNA products are pooled, cleaned up, and amplified with Illumina specific P5/P7 primers with barcodes per plate, pooled, and ready for sequencing. The rest of the cDNA/ADT/DNA amplified product can be used to isolate the cDNA and ADT using solid phase reversible immobilization (SPRI) cell size exclusion. The ADT is then amplified once more with Illumina specific P5/P7 primers with barcodes per plate, pooled, and ready for sequencing. The cDNA is first tagmented with NexteraXT Tn5 and only the 3′ ends are preferentially amplified with custom Illumina specific P5/P7 primers with barcodes per plate, pooled, and ready for sequencing.

### Example 1: Multiomic Investigation of Nucleotide Editing by CRISPR with ADT, Flow Cytometry and Transcriptome Sequencing (MINECRAFT-seq)

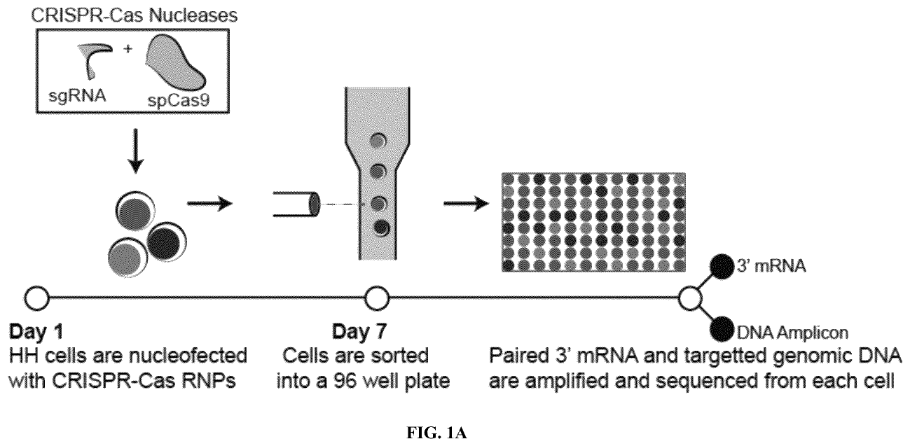

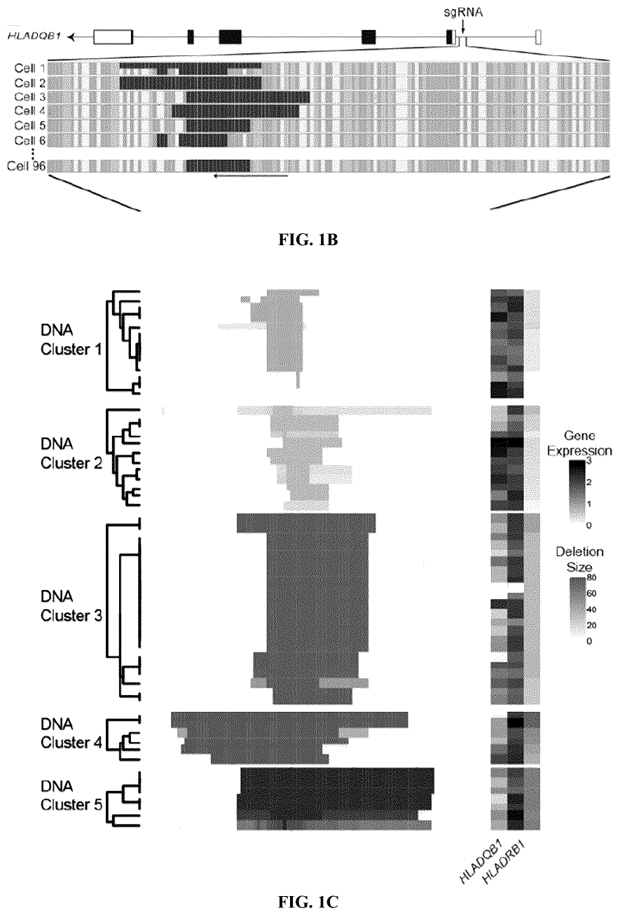

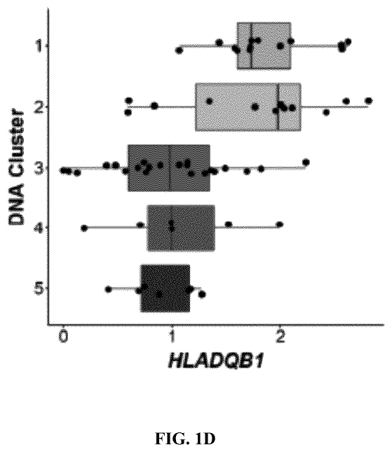

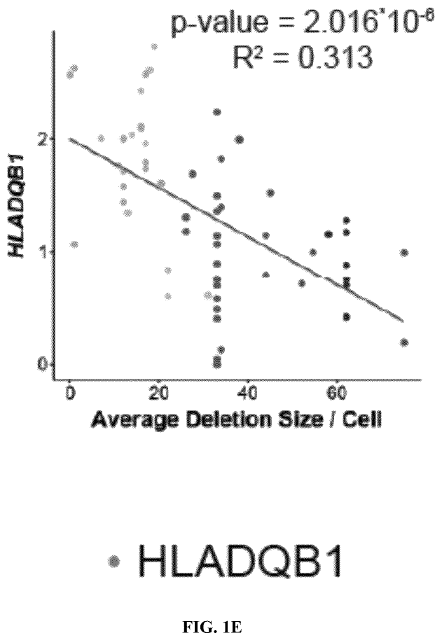

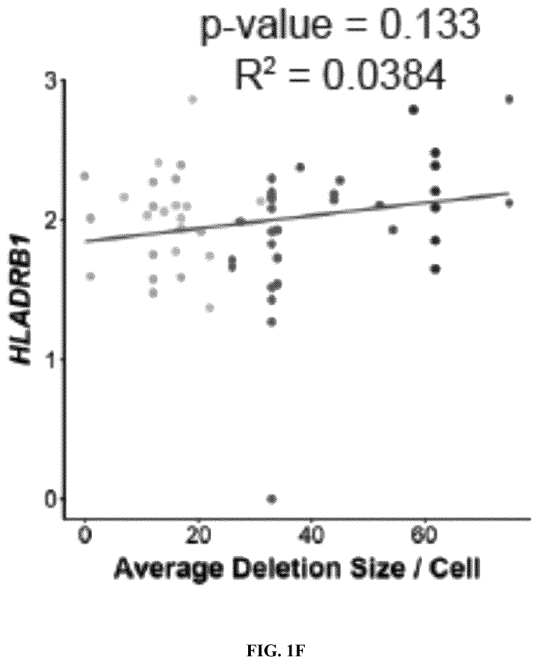

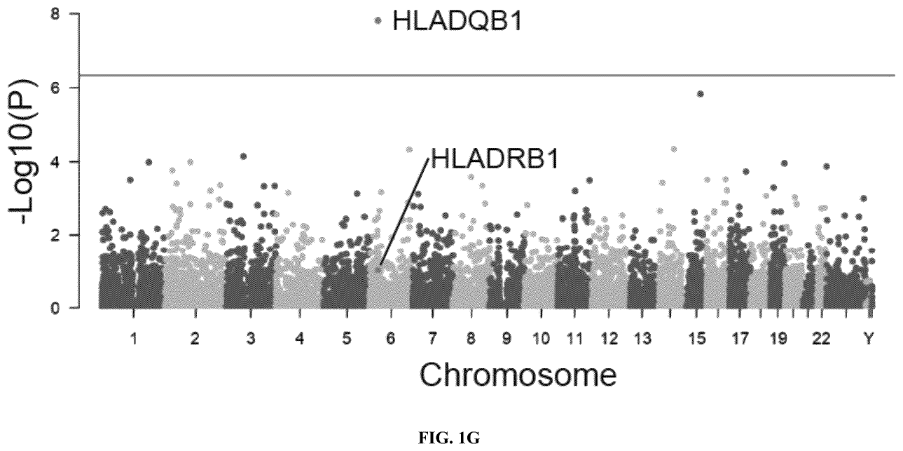

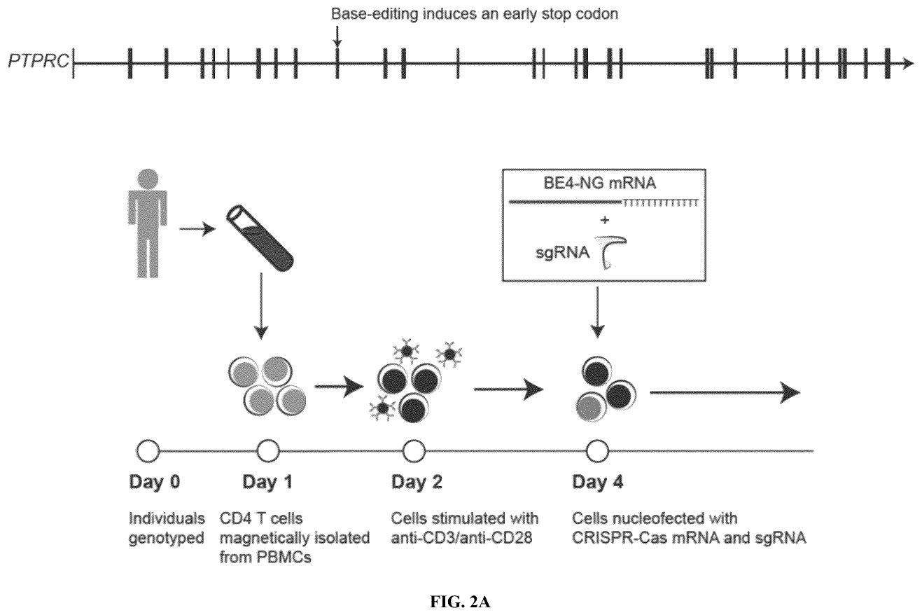

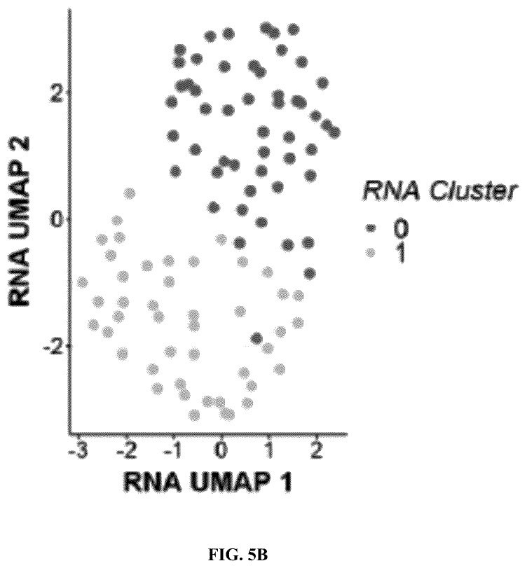

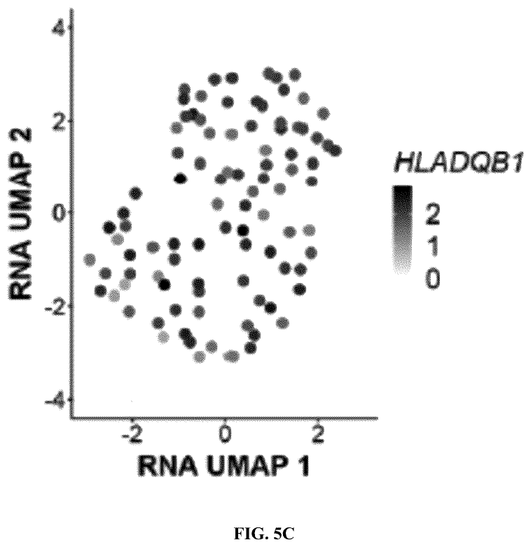

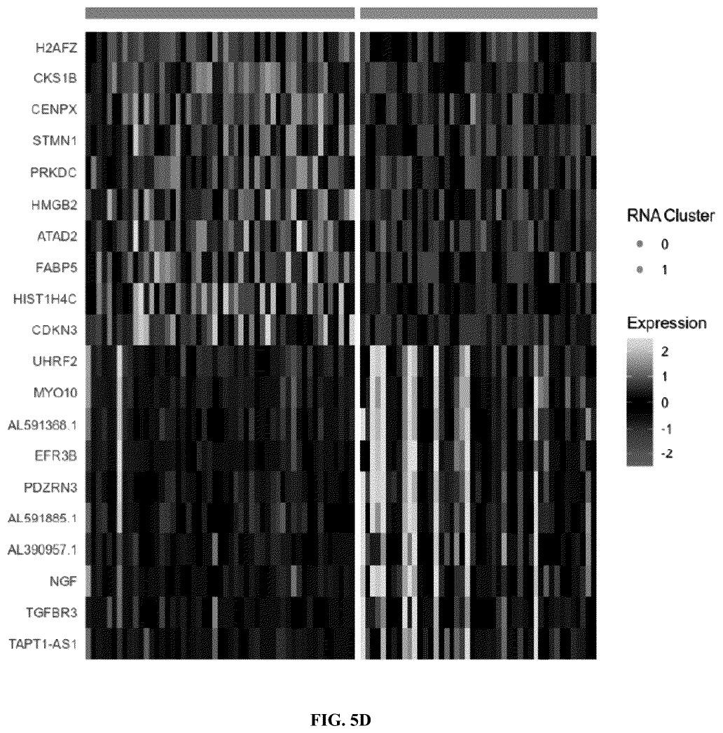

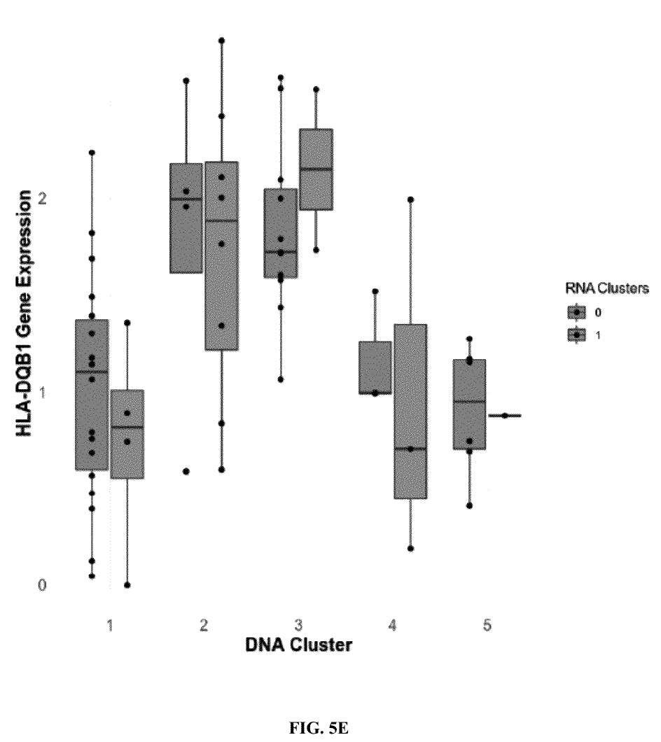

To highlight the utility of a high dimensional multi-modal targeted approach in resolving CRISPR editing outcomes, a modified TARGETseq protocol was applied to a single plate of 96 HH cells edited with CRISPR-Cas9 nucleases targeting a previously validated regulatory region upstream of HLADQBJ (FIG. 1A). In these 96 cells paired genomic DNA and mRNA was recovered from 68 samples filtering on at least 10 aligned genomic DNA reads per cell, greater than 300 mRNA genes per cell and less than 10% mitochondrial gene reads. Genomic DNA amplicons were analyzed around the targeted site from these single cells and enormous heterogeneity in genomic editing was observed. In total 29 unique genotypes were observed that could be grouped into 5 distinct clusters. (FIGS. 1B and 1C). Then mRNA expression levels were tested in each individual cell by calculating the number of unique molecular identifiers per gene allowing for barcode error correction using STARSolo. HLADQB1 expression normalized per cell and scaled across all cells was different for the distinct clusters of DNA edits (p=4.05e-05, ANOVA). It was observed that HLADQB1 was associated with average deletion size after adjusting for the 21815 tested genes (p<4.58e-07=0.01/21815, FIGS. 1D-1G, and FIGS. 5A-5E). It was observed that none of the other genes were associated with deletion size, demonstrating the precision with which this single cell assay can define a target gene in a limited number of cells.

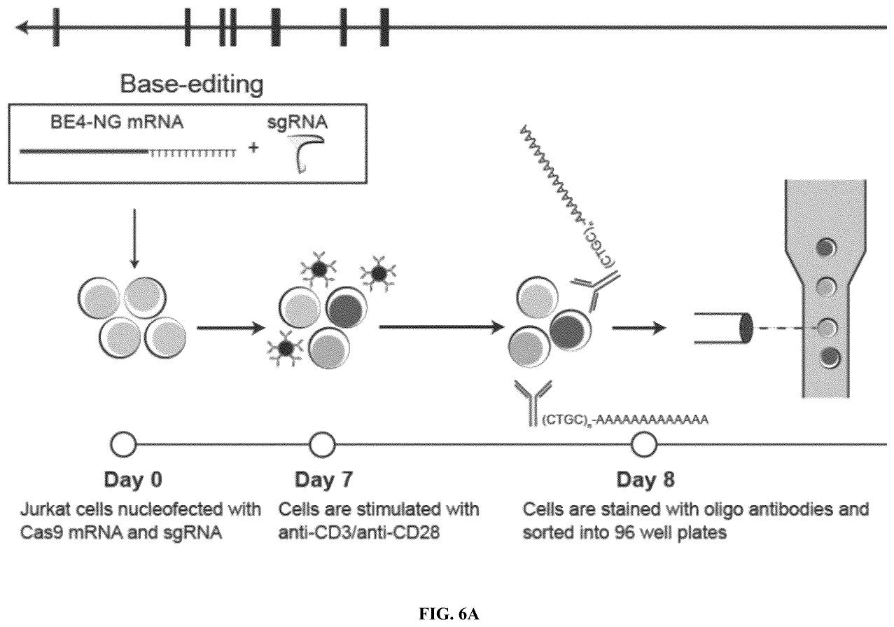

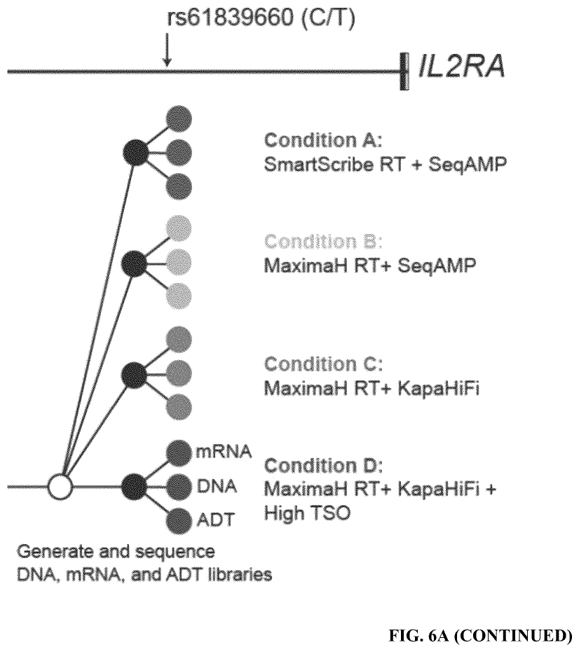

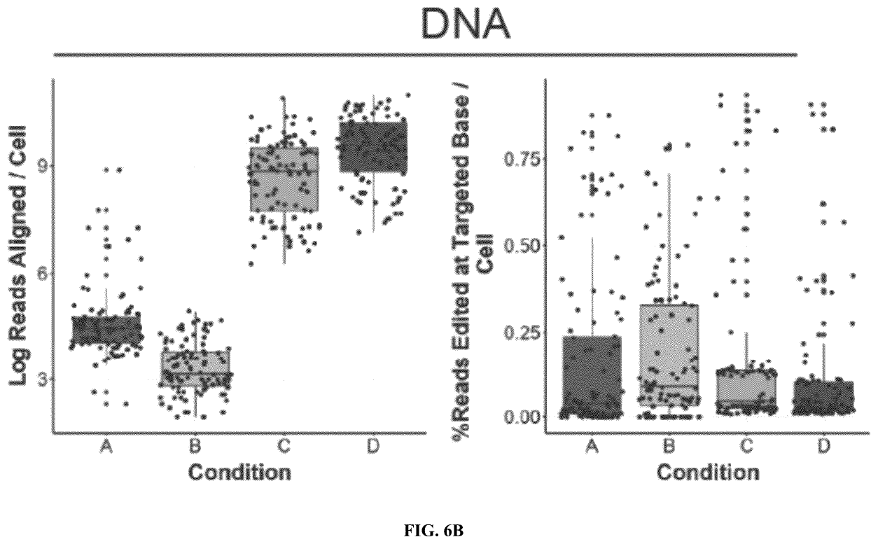

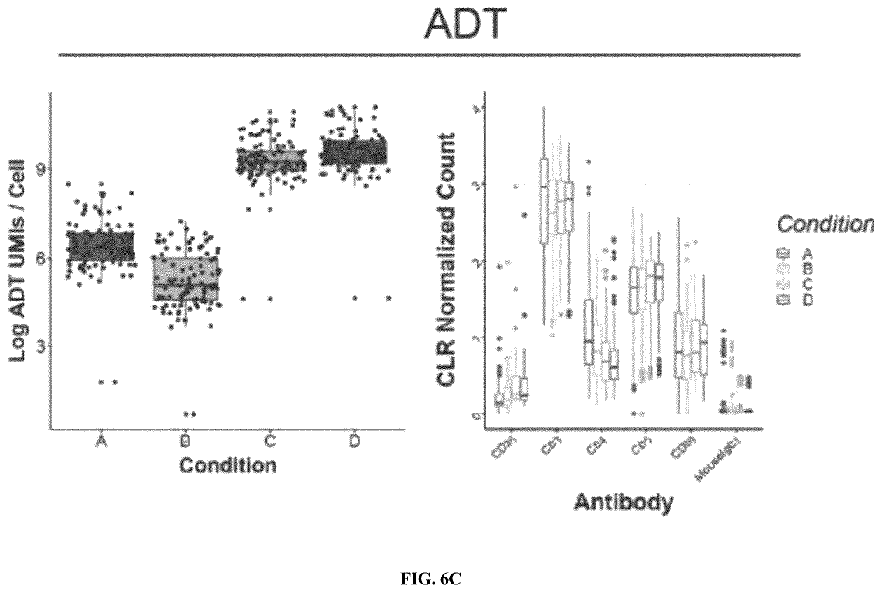

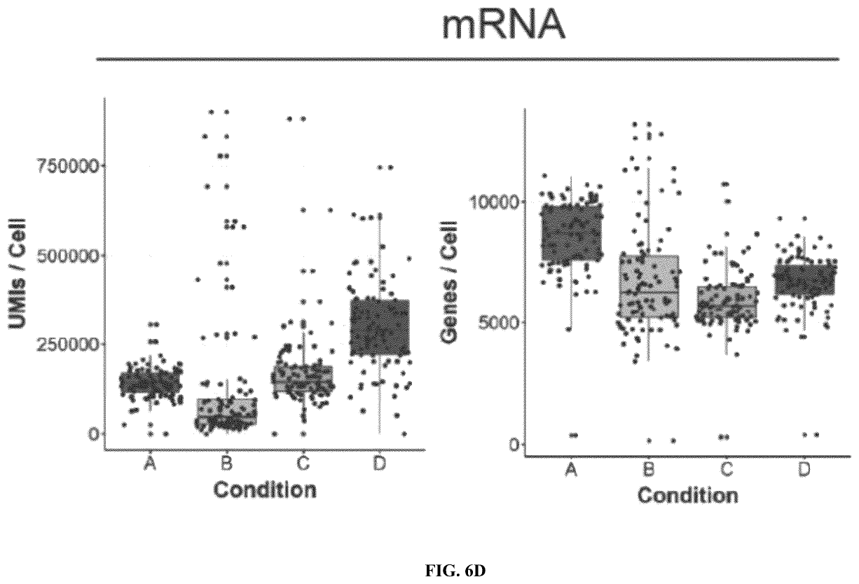

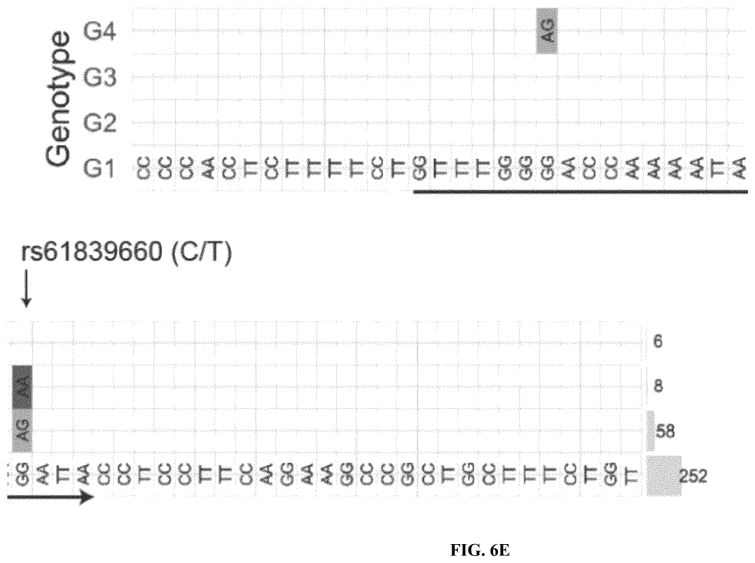

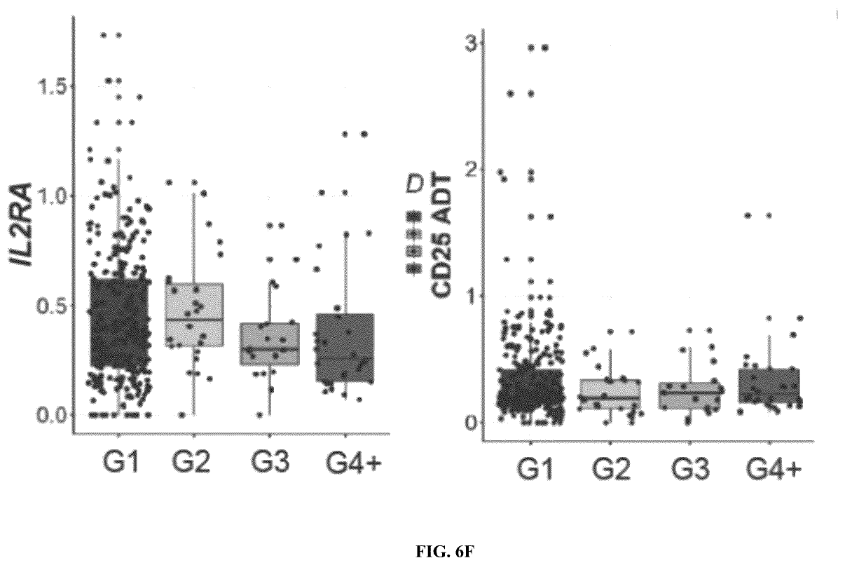

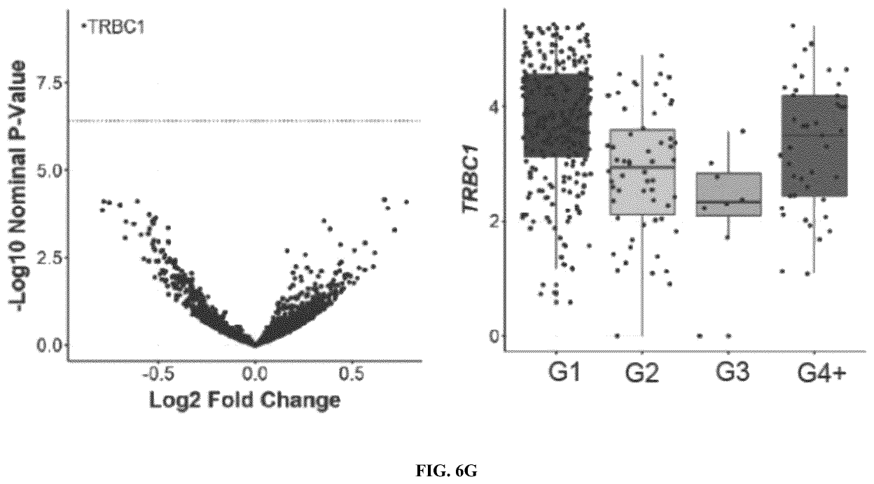

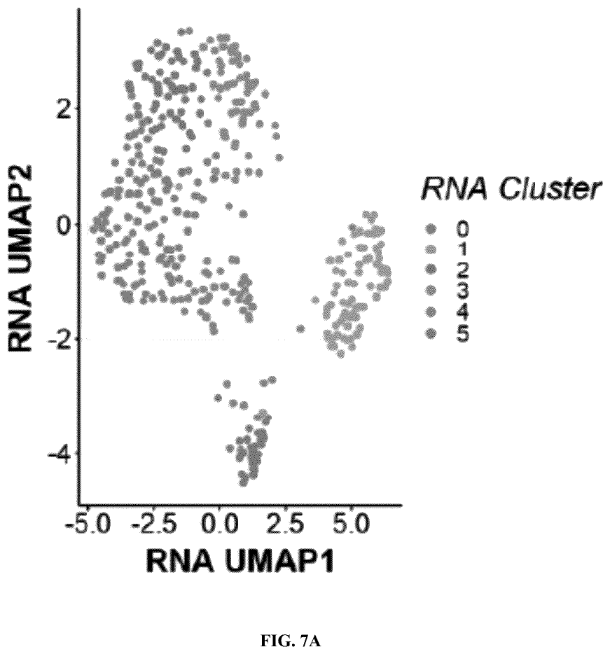

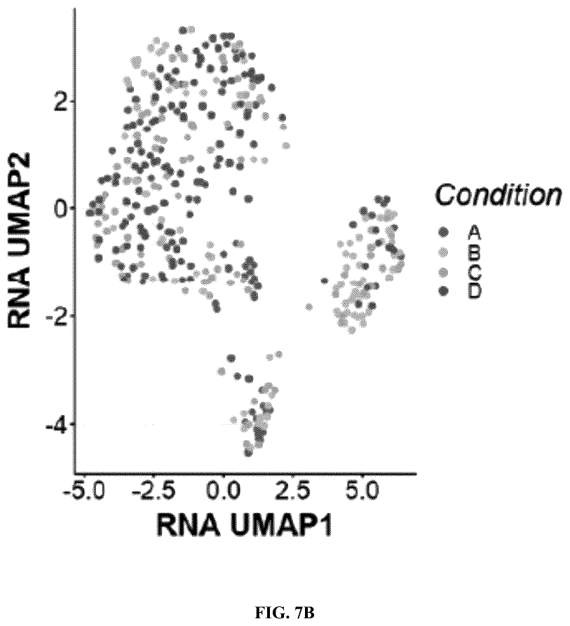

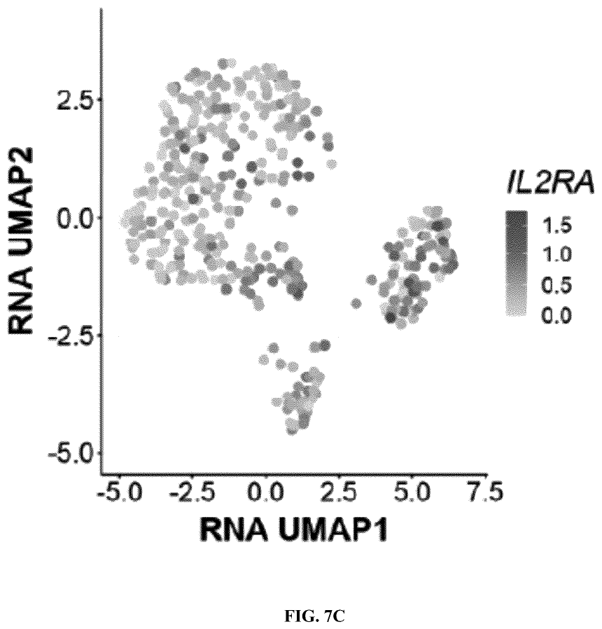

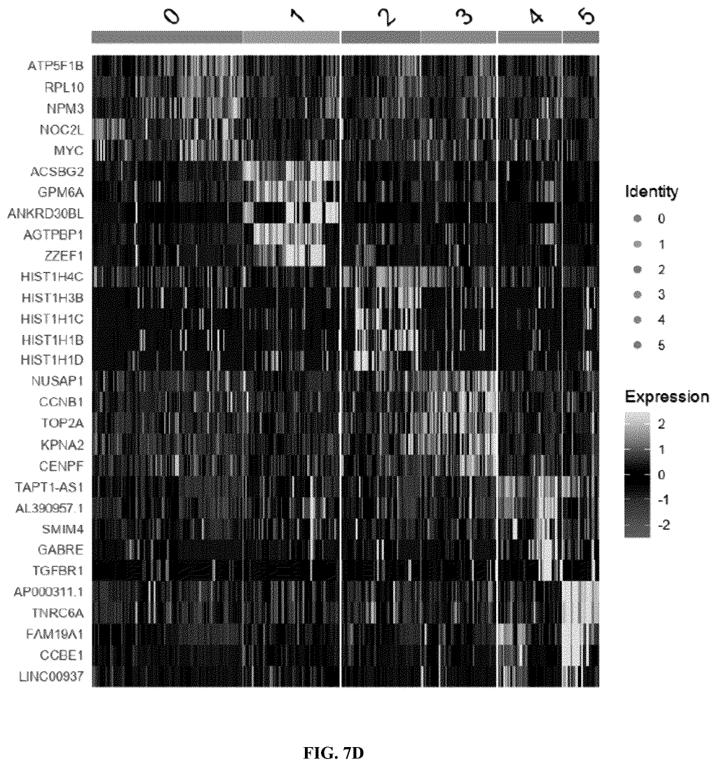

It was recognized that cell surface protein antibody assays would be essential to understand protein expression and to characterize cell states. For immune cell populations, phenotyping cells with antibody derived tags has proven to be essential to resolving single cell immune populations. To improve on current plate-based methodologies and incorporate this critical modality, several multi-omic protocols were tested (FIGS. 6A-6G). CRISPR-Cas base-editors (BE4-NG) were used to target a variant, rs61839660, in IL2RA to investigate recoverability of all three modalities: genomic DNA amplicons, 3′mRNA, and antibody-derived tags (ADT) expression. From optimizations, it was noted that a Smart-seq3-like approach with MaximaH, KapaHIf1, and increased Template Switch Oligo (TSO) concentration resulted in good recoverability of all three modalities as measured by the number of recovered and aligned genomic DNA reads per cell, unique molecular identifiers (UMIs) of ADT per cell, and UMIs of genes per cell (FIGS. 6A-6G). Pooling all the data together, a correlation was not found between editing at rs61839660 and expression of either IL2RA or CD25 ADT (FIGS. 6A-6G and 7A-7D).

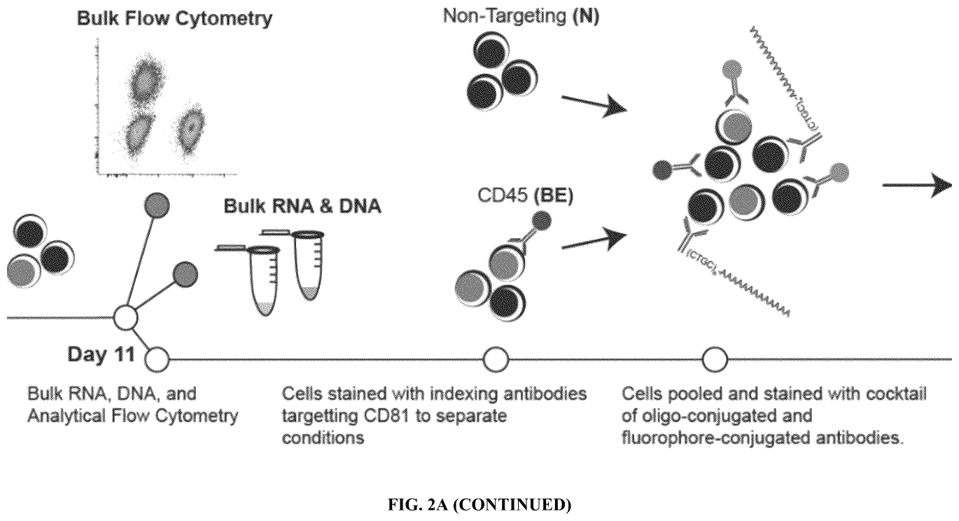

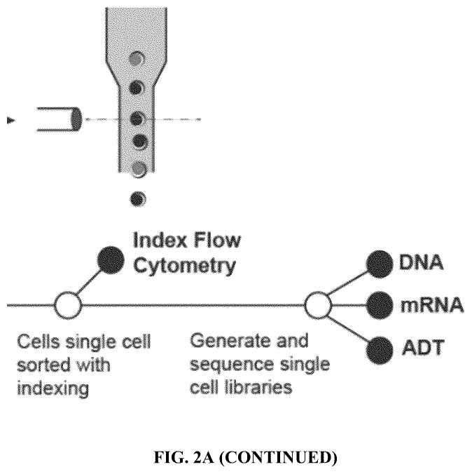

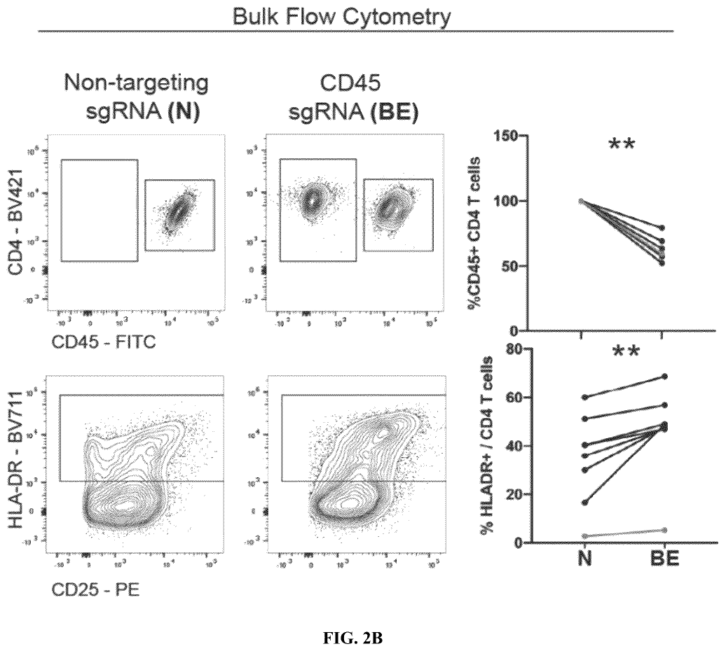

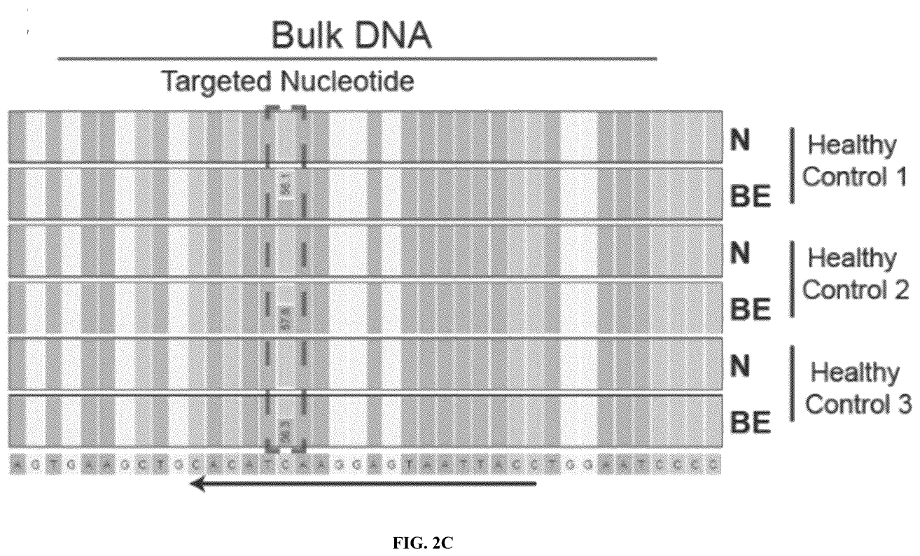

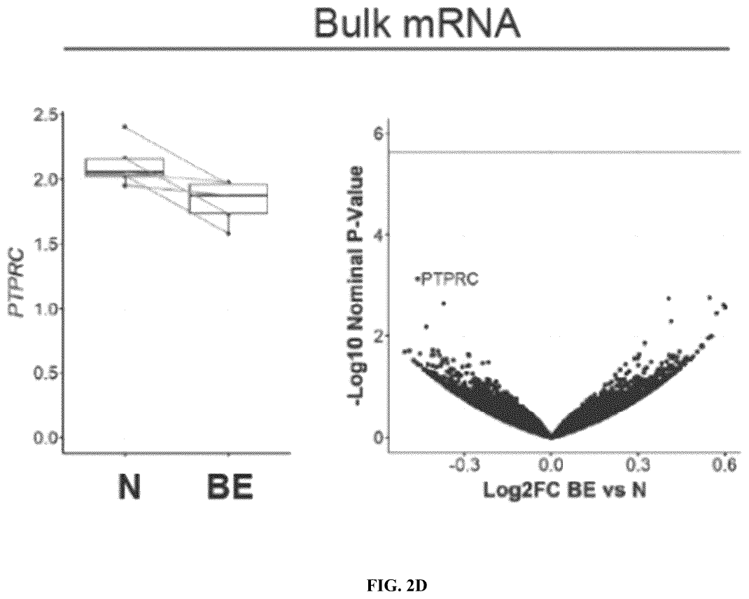

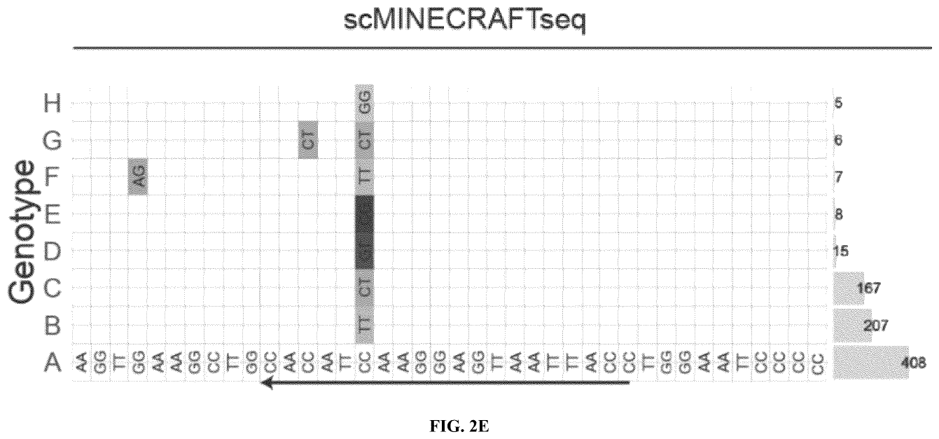

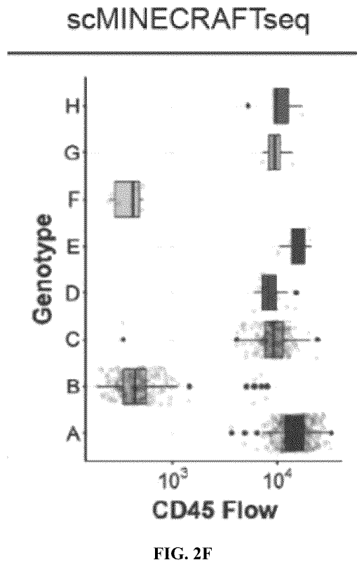

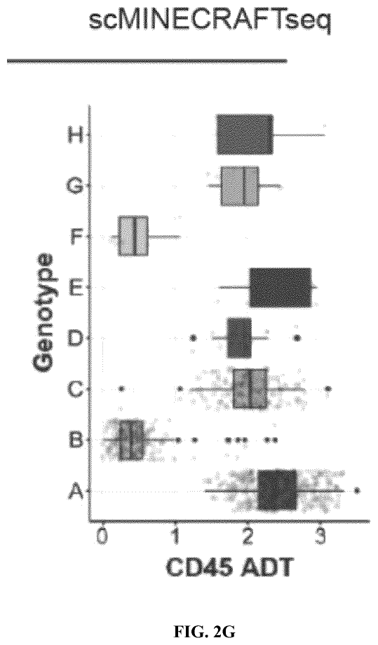

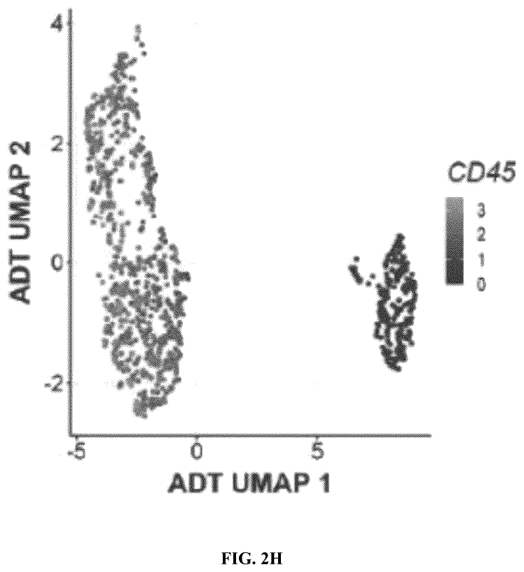

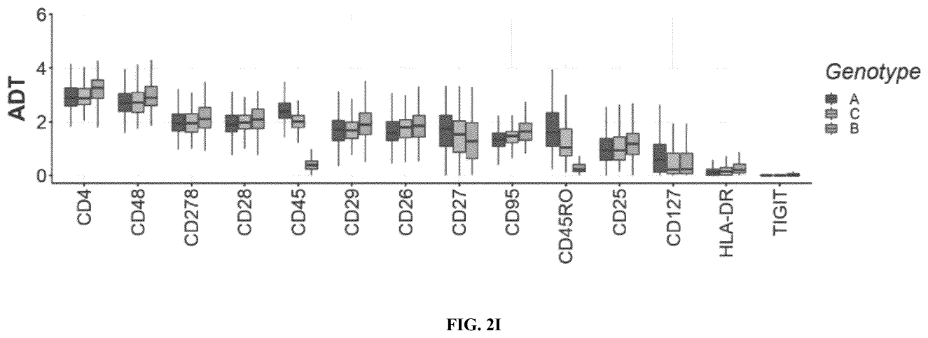

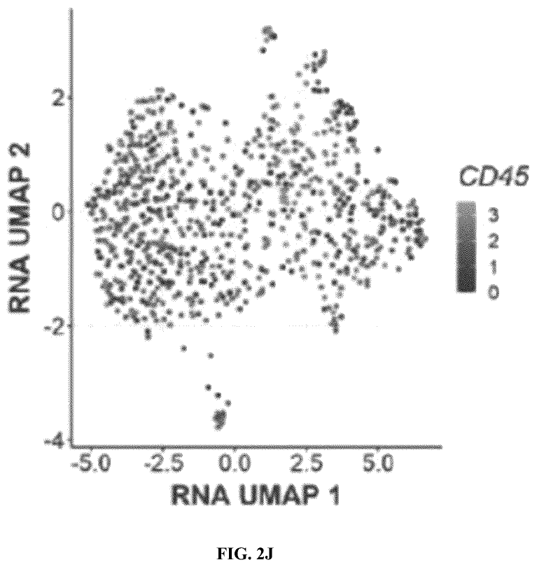

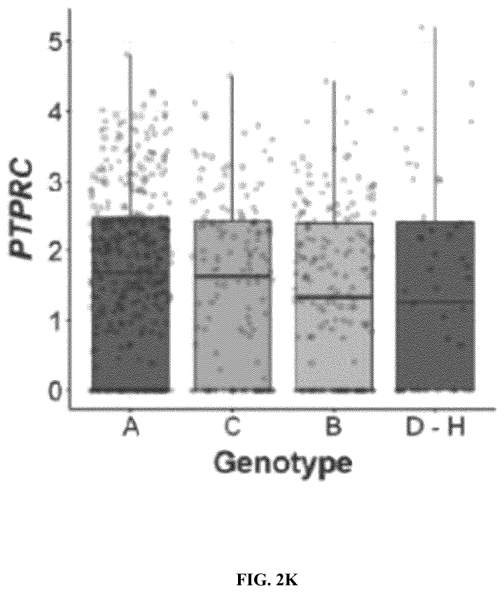

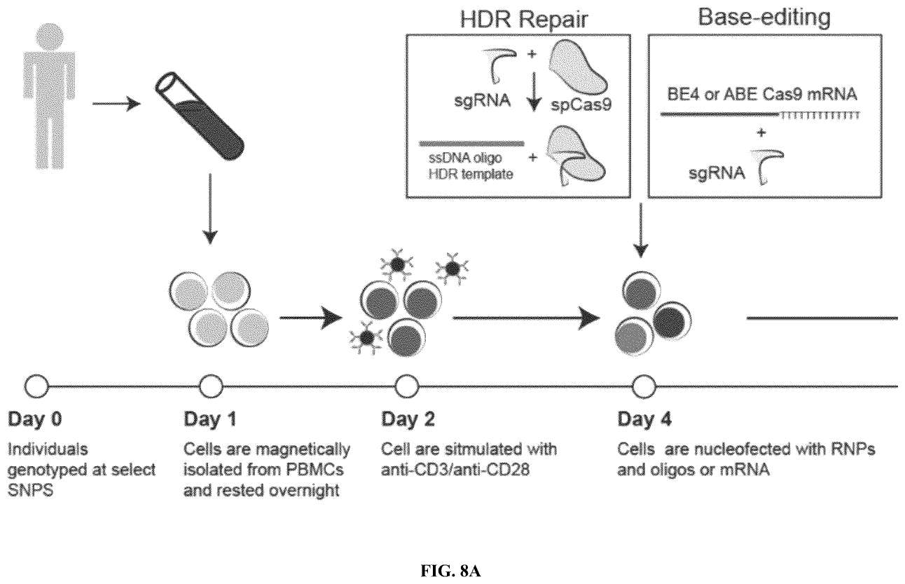

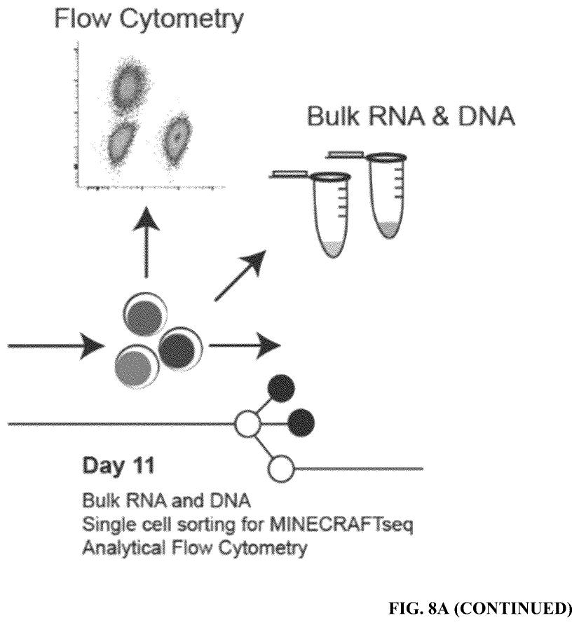

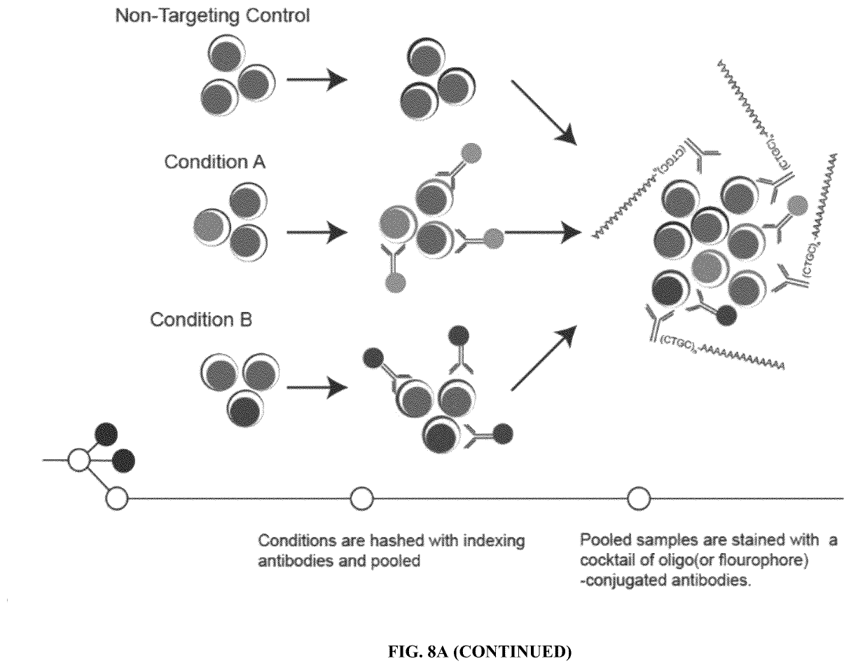

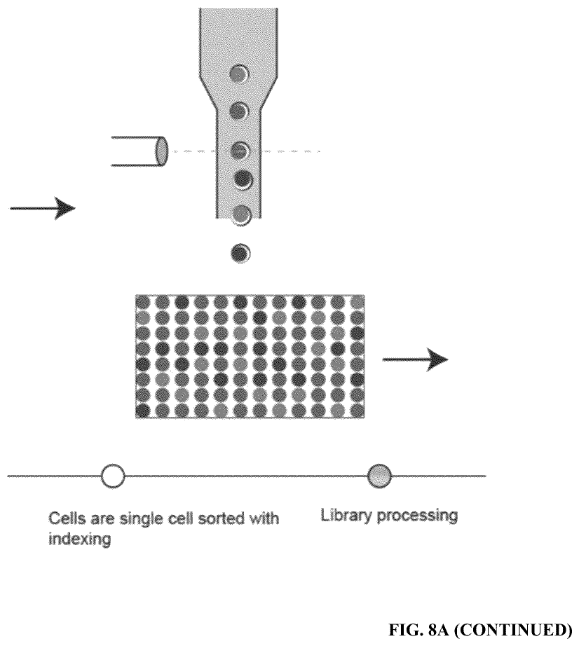

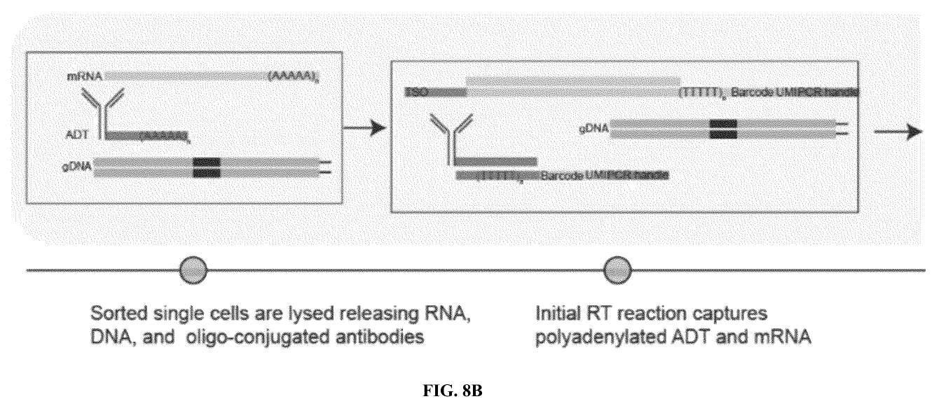

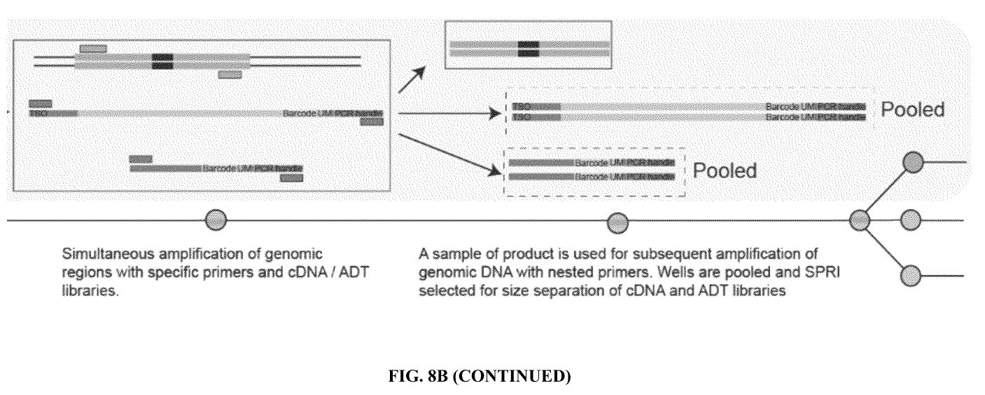

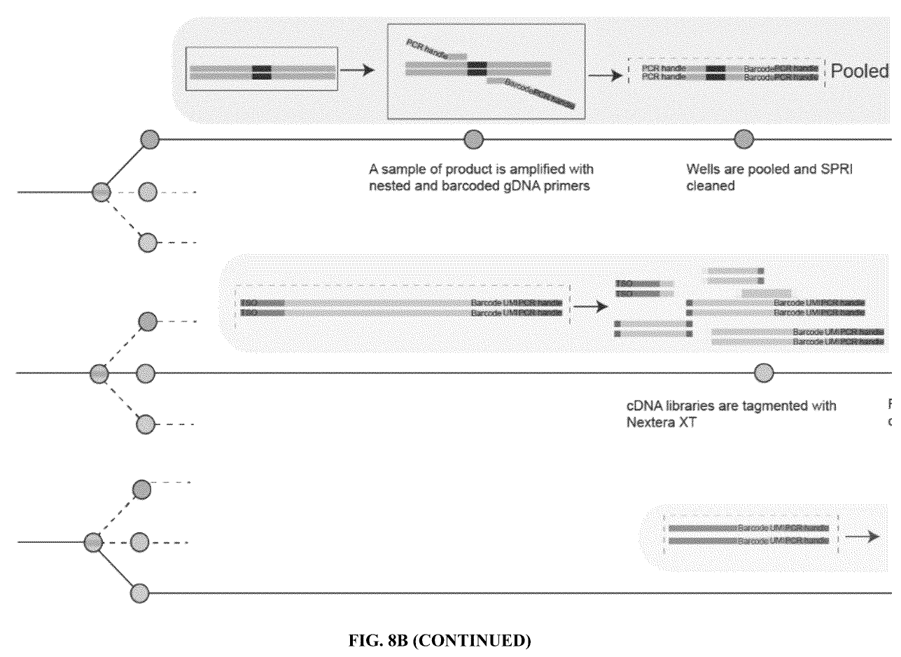

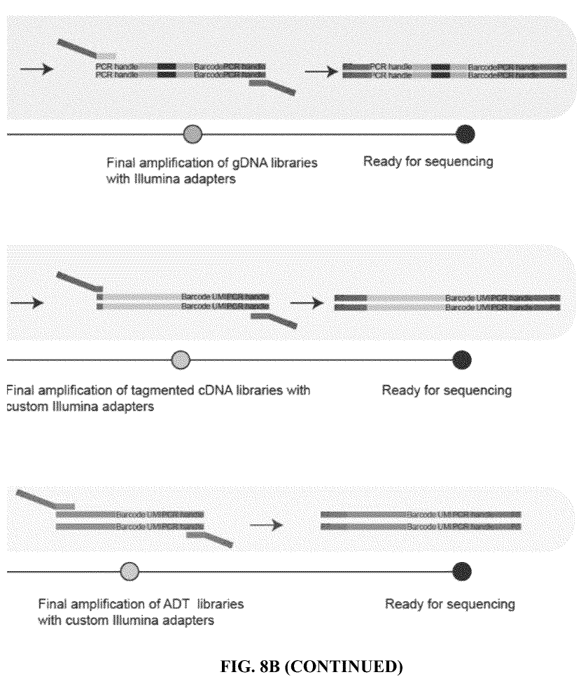

The examination of disease loci and variants in primary cells of interest represents the next frontier in functional genomics. To investigate the effects of CRISPR editing in primary immune cells, all three single cell modalities, genomic DNA, ADT, and 3′ mRNA, were integrated with index sorting to create a multi-omic plate-based single cell approach termed MINECRAFT-seq (Multiomic Investigation of Nucleotide Editing by CRISPR with ADT, Flow cytometry and Transcriptome sequencing) (FIGS. 2A, 8A, and 8B). Critically, this methodology allowed for mixing of conditions reducing batch effects and does not require any expensive droplet generation equipment.

### Example 2: Application of Multiomic Investigation of Nucleotide Editing by CRISPR with ADT, Flow Cytometry and Transcriptome Sequencing (MINECRAFT-Seq) to CD4 T Cells

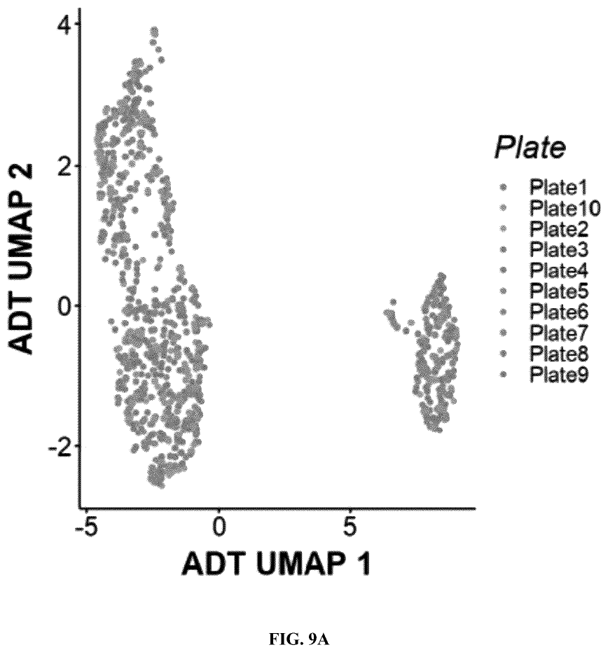

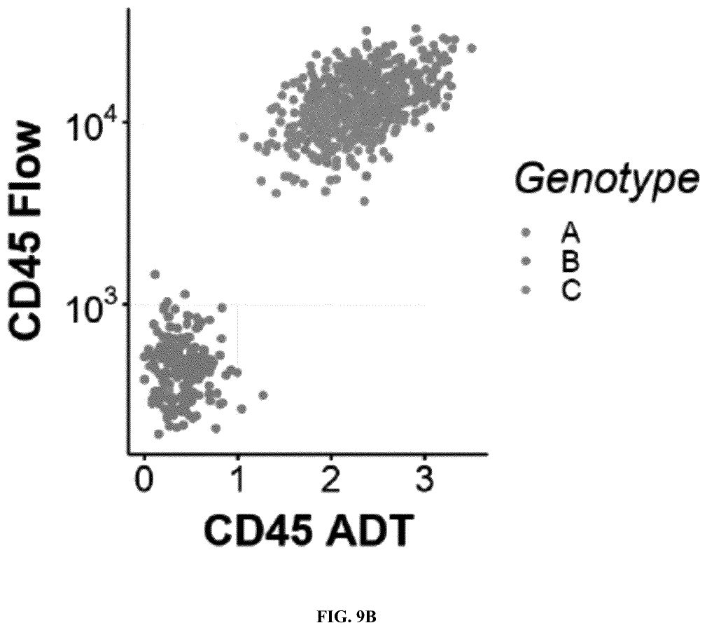

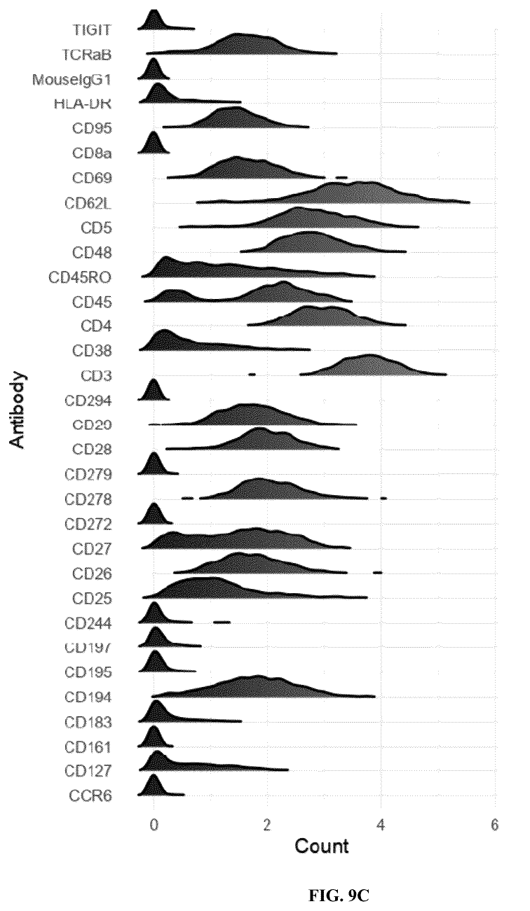

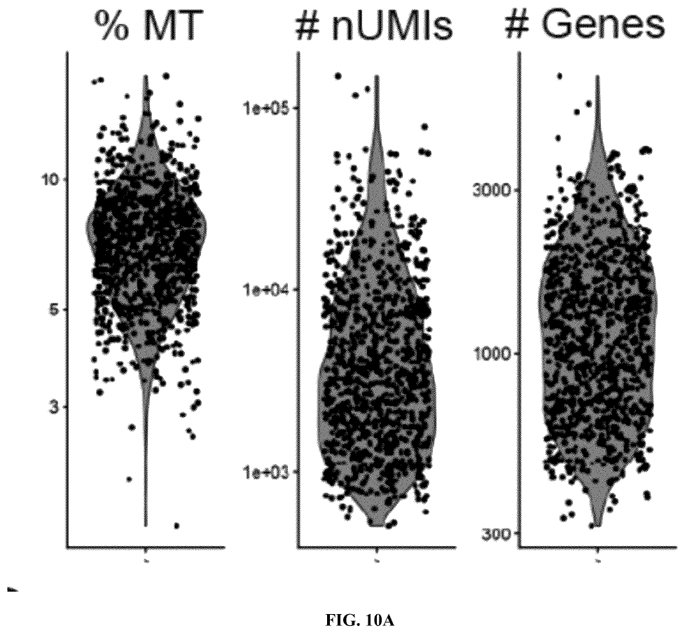

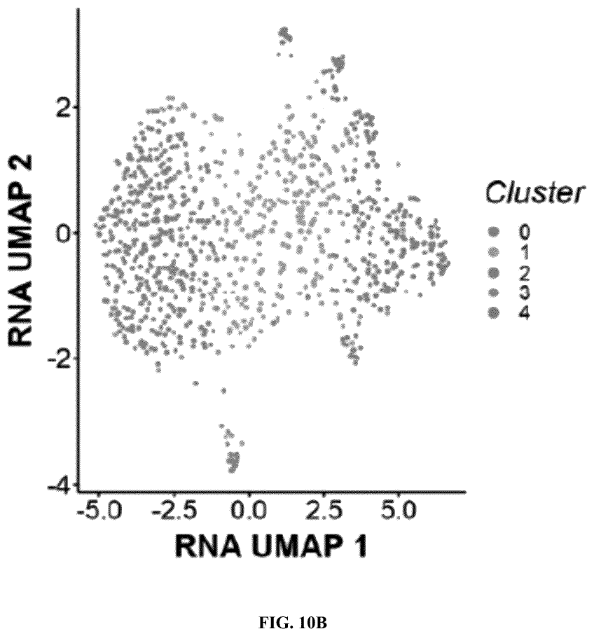

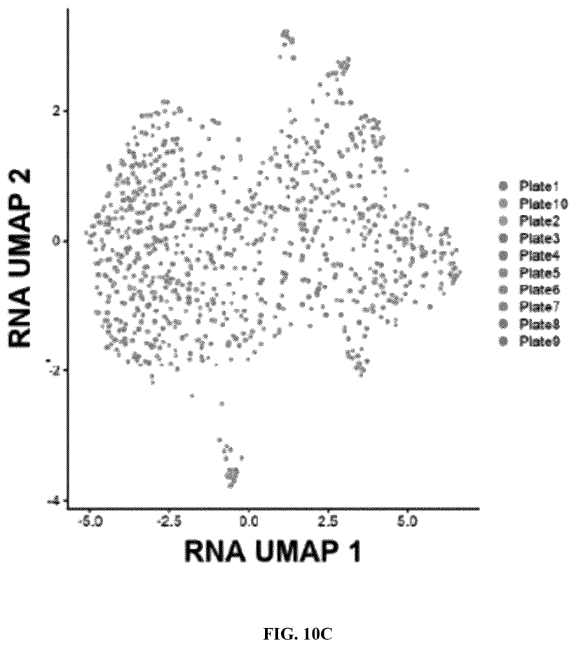

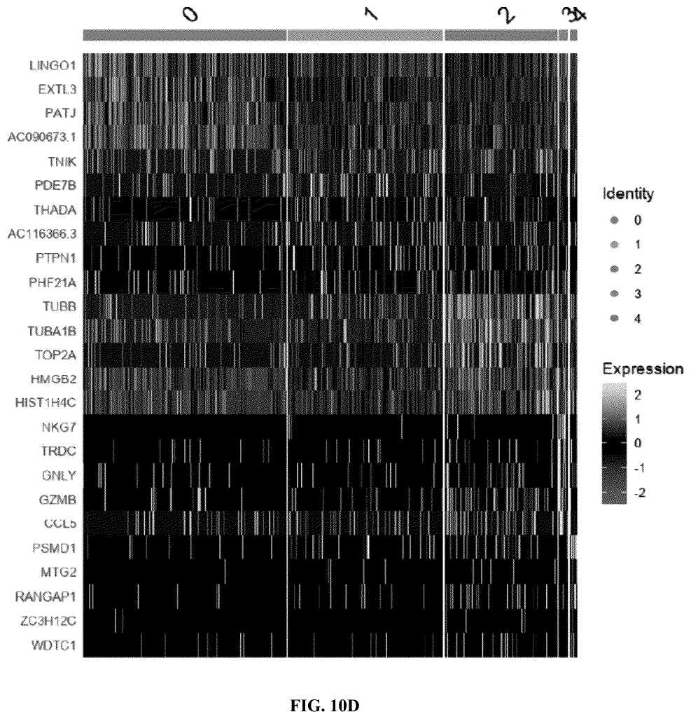

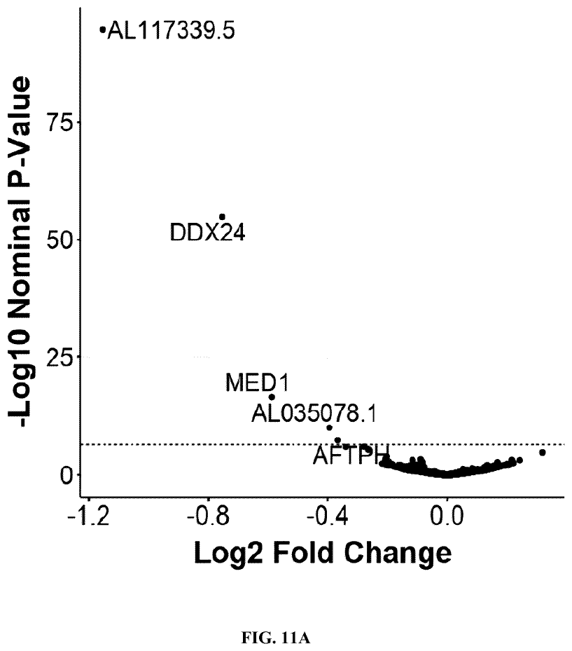

To showcase the utility of this approach in primary CD4 T cells, CRISPR-Cas base editors were used to induce an early stop codon in PTPRC and processed 960 cells from one healthy individual using single cell MINECRAFT-seq (FIGS. 2A-2K). Genomic DNA was filtered on at least 10 reads per cell and aligned to a reference amplicon sequence using CRISPResso2. The mRNA counts were aligned and calculated with STARSolo and ADT counts calculated using kallisto KITE. For comparison bulk analysis from was also conducted additional healthy individuals. It was observed that CRISPR base-editing in PTRPC caused a substantial knockout of CD45 and skewed cells towards late-stage activation, but did not cause changes in overall gene expression (FIGS. 2B-2D). Using the multi-omic single cell approach, 8 unique genotypes (with at least four cells) were identified, including bystander outcomes (FIG. 2E). Of the edited cells some had no introduced edits, and some had the intended GGG genotype change. These unique genotypes correlate to varying levels of CD45 protein expression and identified a clear heterozygote effect (p<2e-16, ANOVA, FIGS. 2F and 2G). Analysis of all ADT markers revealed a distinct knockout cluster (FIGS. 2H, 2I, and FIGS. 9A-9C). Furthermore, a strong correlation was shown between flow based cell surface expression and a sequencing readout (FIGS. 9A-9C). Clustering of mRNA, unlike the ADT, did not identify a unique knockout cluster that was supported by only a modest and insignificant decrease in PTPRC (FIGS. 2J-2K and FIGS. 10A-10D). Differential gene expression at the dosage of the targeted base (comparing genotypes A, C, & B) did reveal broader expressional changes suggesting a subtle change in cell state that could not have been identified in bulk data (FIGS. 11A-11D).

### Example 3: Application of Multiomic Investigation of Nucleotide Editing by CRISPR with ADT, Flow Cytometry and Transcriptome Sequencing (MINECRAFT-Seq) to Investigate Causal Variants in Disease

Capturing gDNA, cell surface protein expression, and mRNA from single cells was effective in revealing heterogeneity in CRISPR editing and inferring phenotypic outcomes. This presented a rare opportunity to study disease-associated variants directly in the primary cell of interest. For a number of autoimmune disorders, that cell type is CD4 T cells. Recent work fine-mapping autoimmune loci has identified potentially causal variants shared in Type 1 Diabetes and Rheumatoid Arthritis with two loci in particular, UBASH3A and IL2RA. Four variants in UBASH3A and three variants in IL2RA were selected for functional follow up using single cell MINECRAFT-seq in primary CD4 T cells.

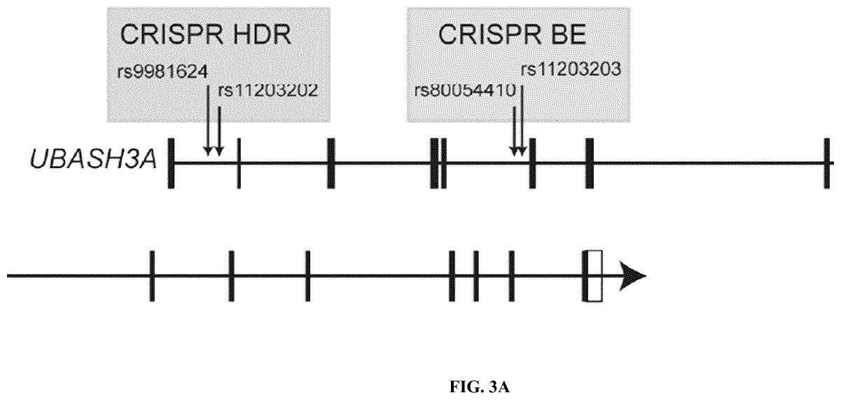

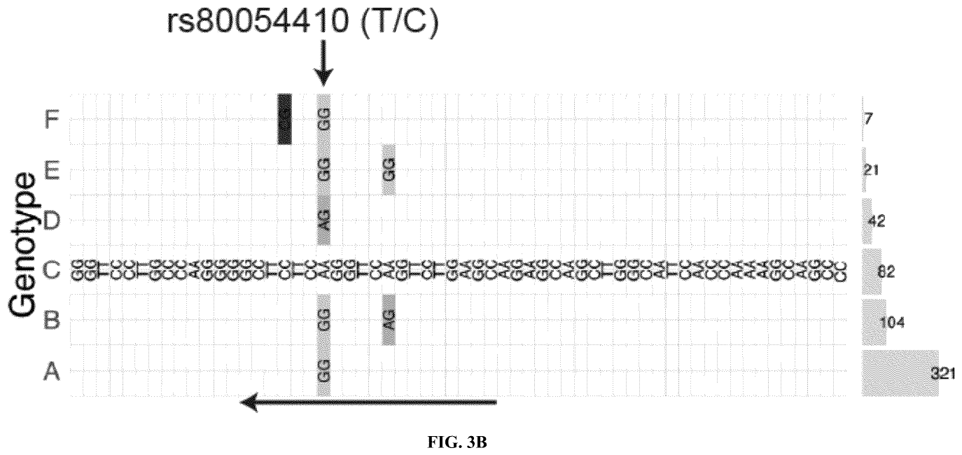

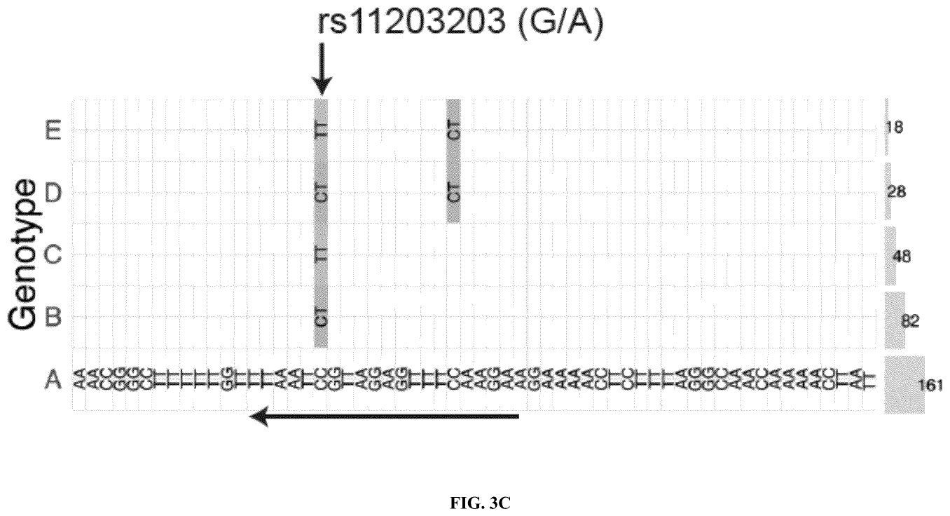

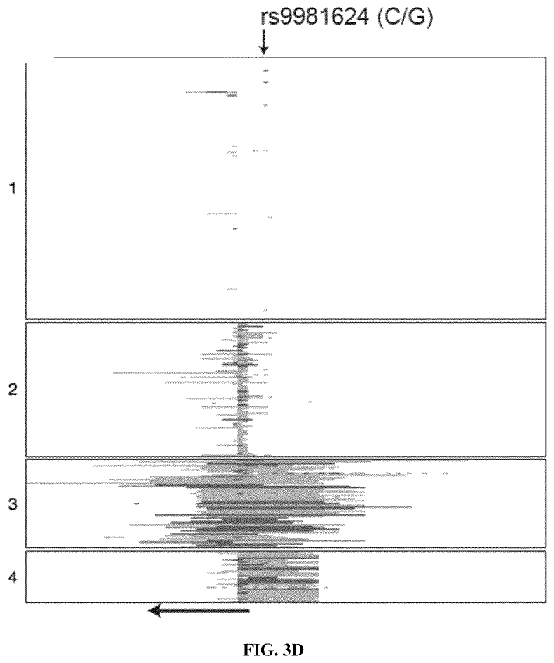

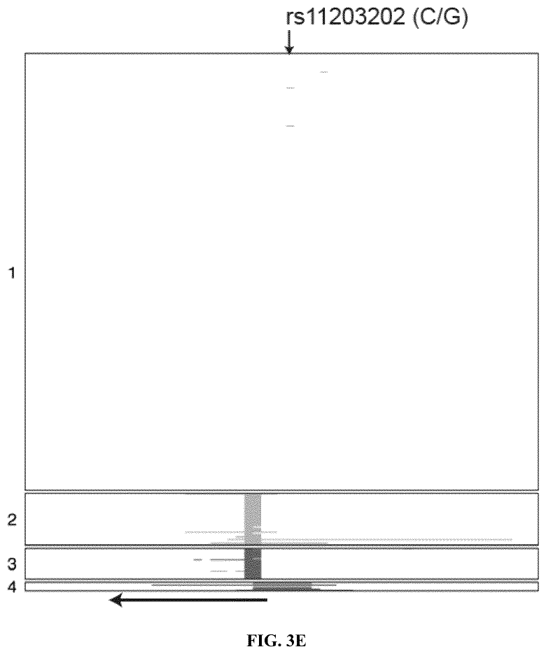

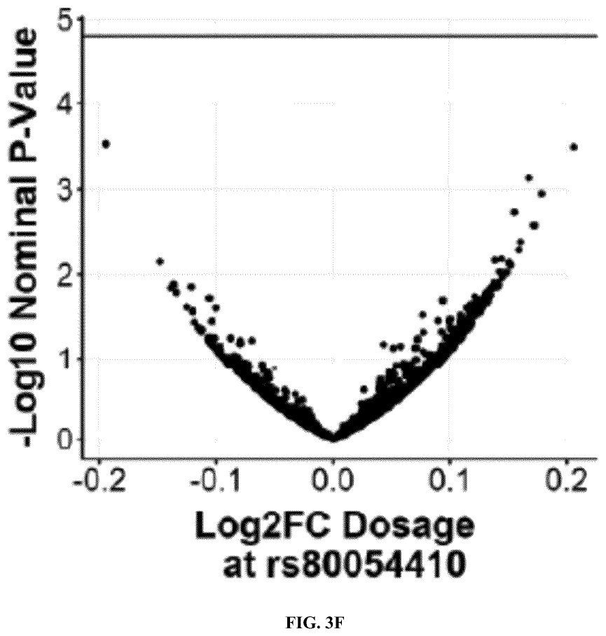

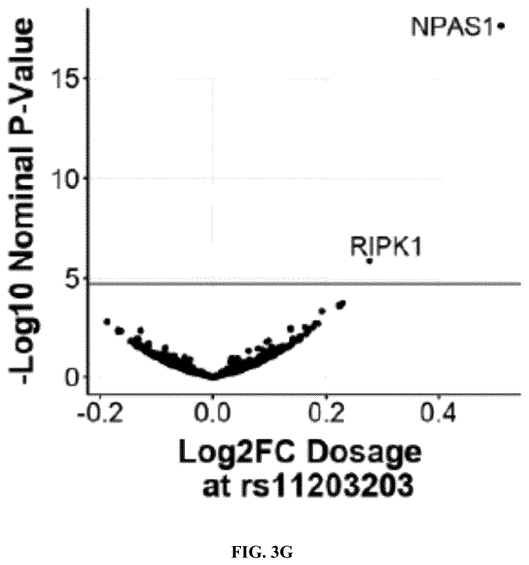

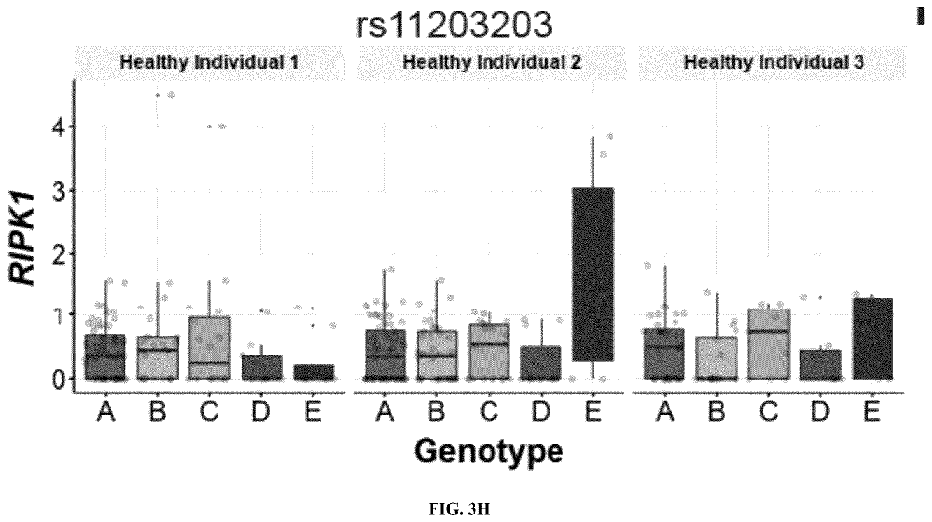

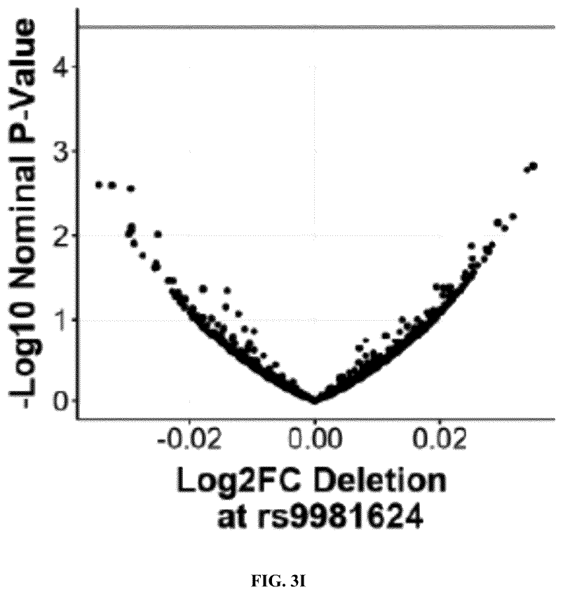

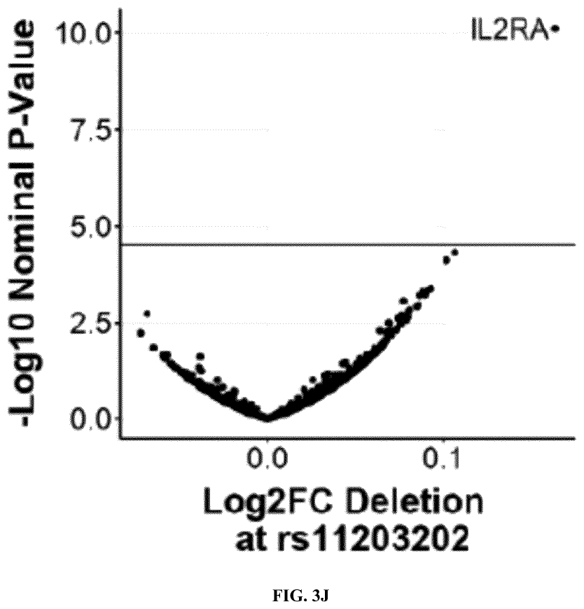

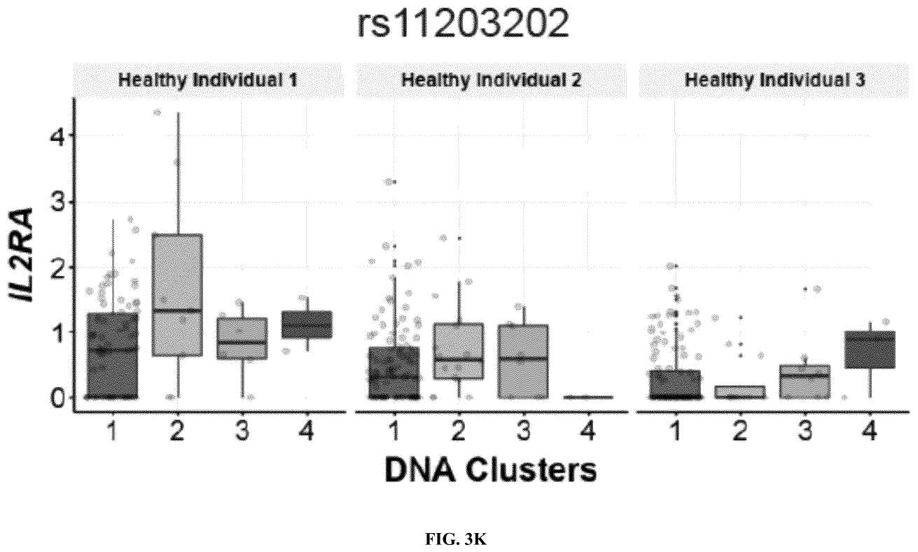

UBASH3A is a ubiquitin associated protein that likely regulates T cell simulation through the T cell receptor (TCR). Knockout of Ubash3a enhances signaling capacity with increased proliferation and IL-2 expression. To investigate all four potentially causal variants in UBASH3A CRISPR-Cas base-editing and HDR repair tools were applied (FIG. 3A and FIGS. 8A and 8B).

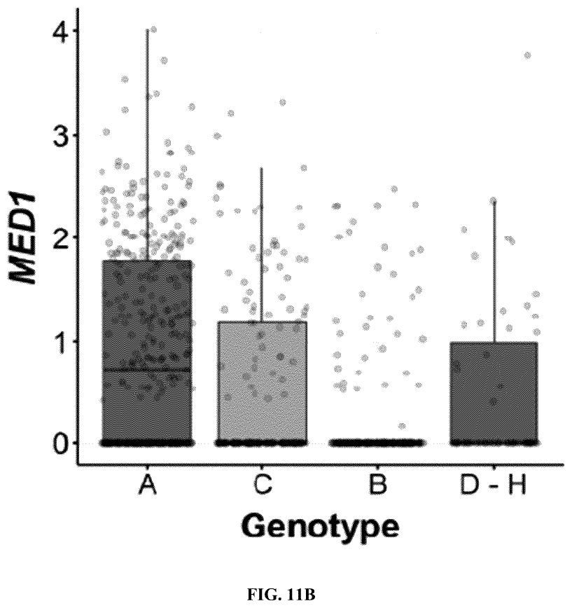

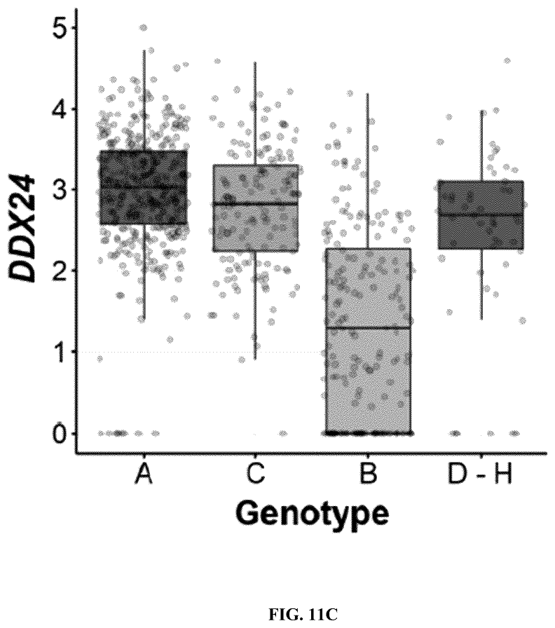

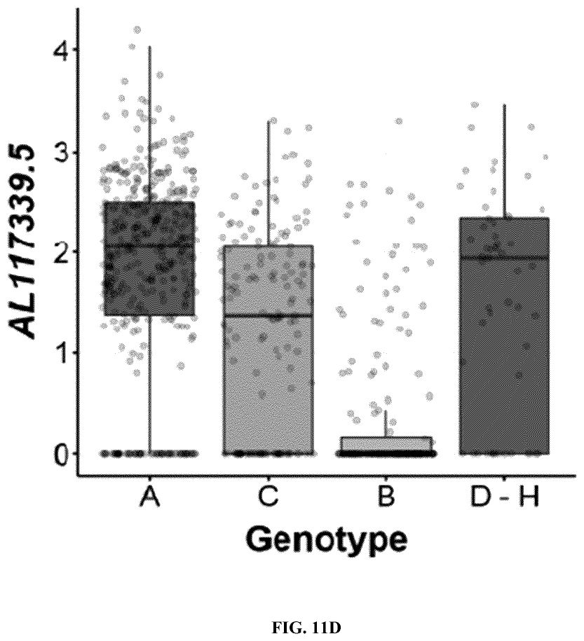

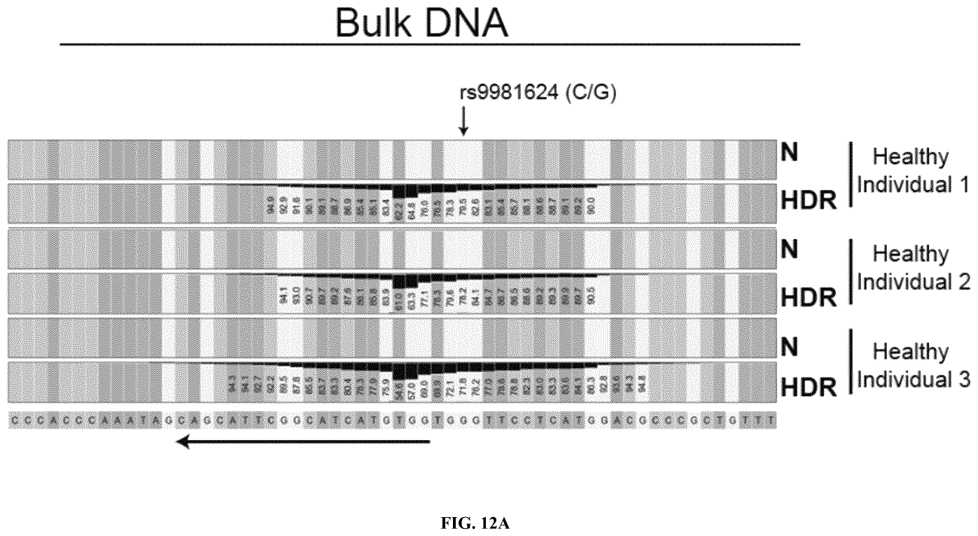

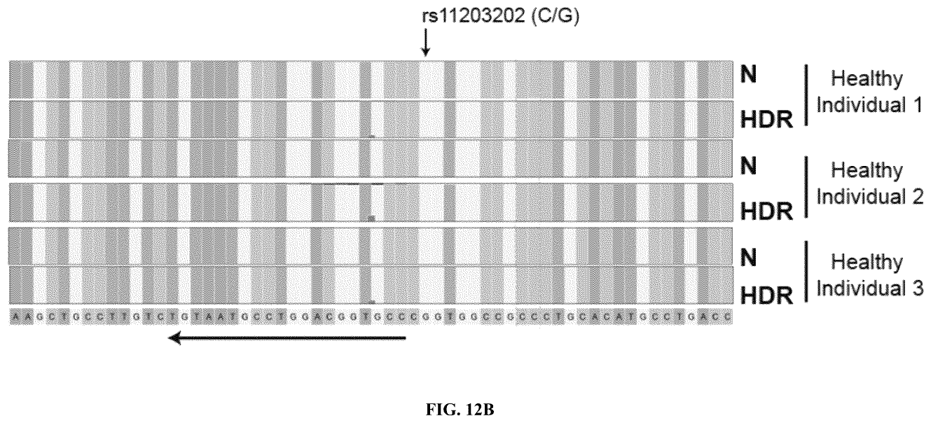

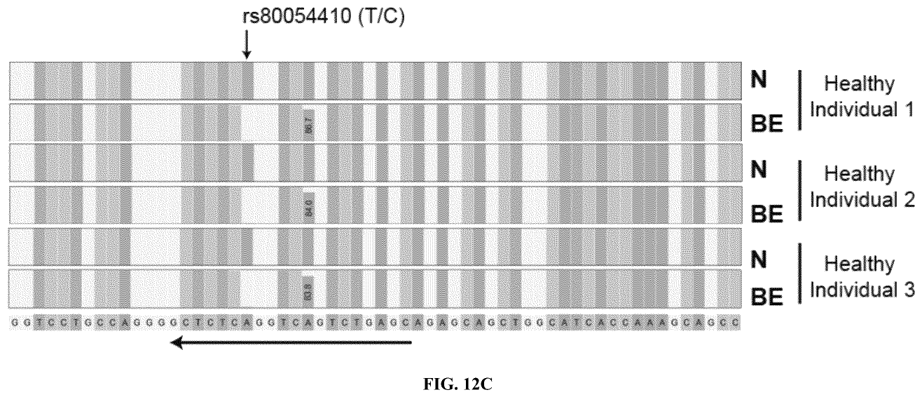

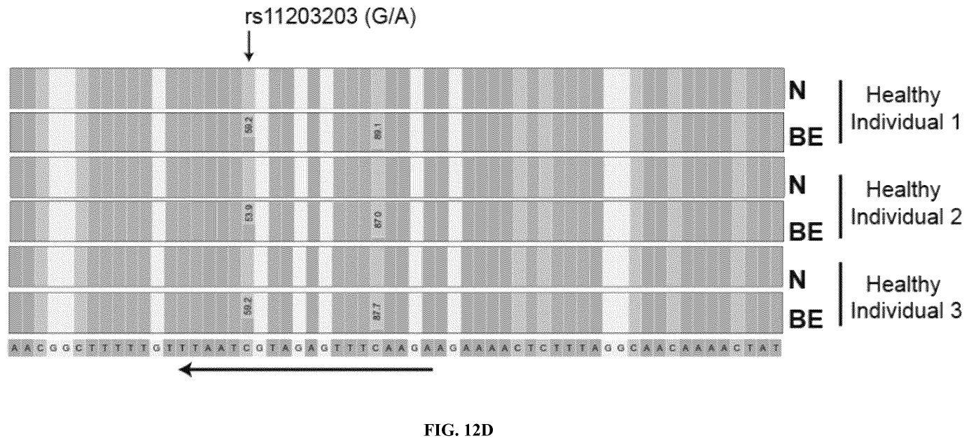

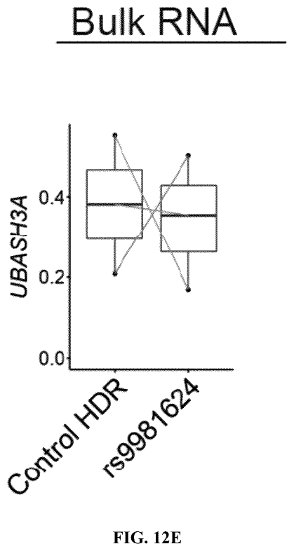

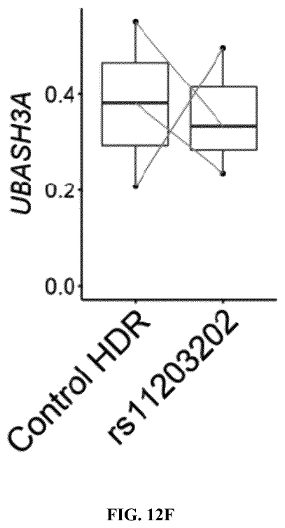

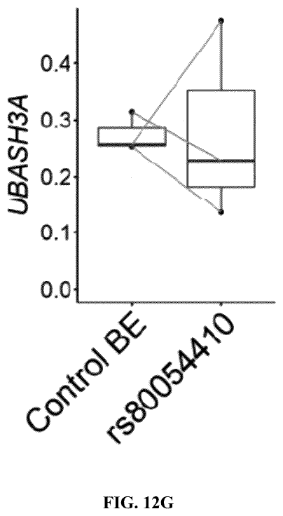

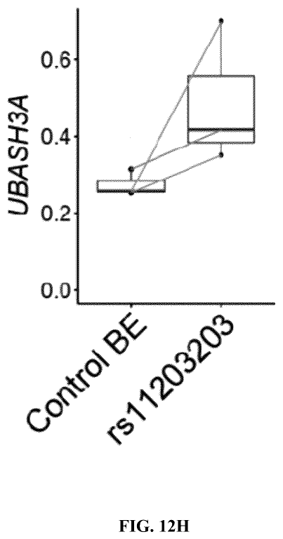

Analysis of bulk genomic DNA, mRNA, and flow cytometry data identified differences in editing efficiency and evidence of predominant indels (insertion and deletions) in HDR targeted samples (FIGS. 12A-12J). Targeting only rs11203203 and not any other variants resulted in a nominal increase in UBASH3A expression (FC=0.83, p=0.1791 Welch t-test) that was not significant after genome wide correction (FIGS. 12A-12J).

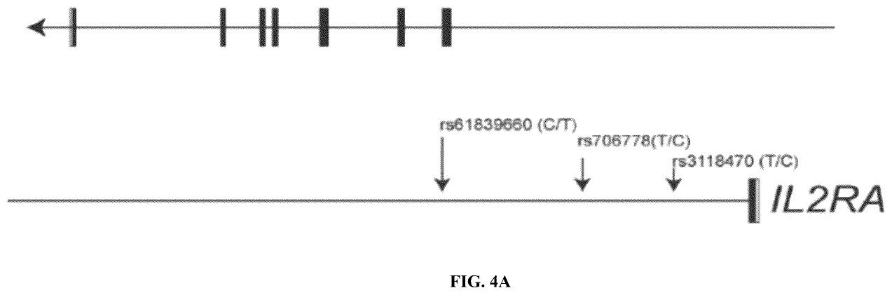

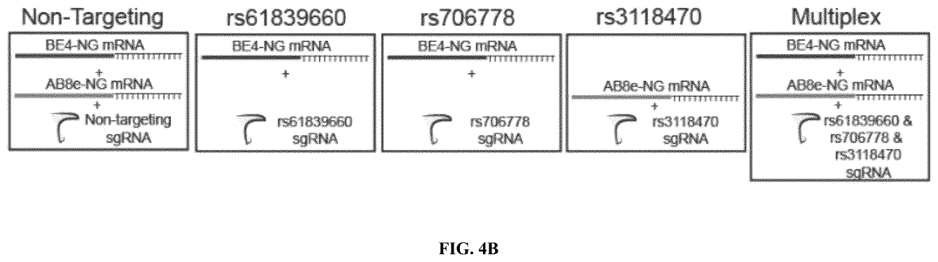

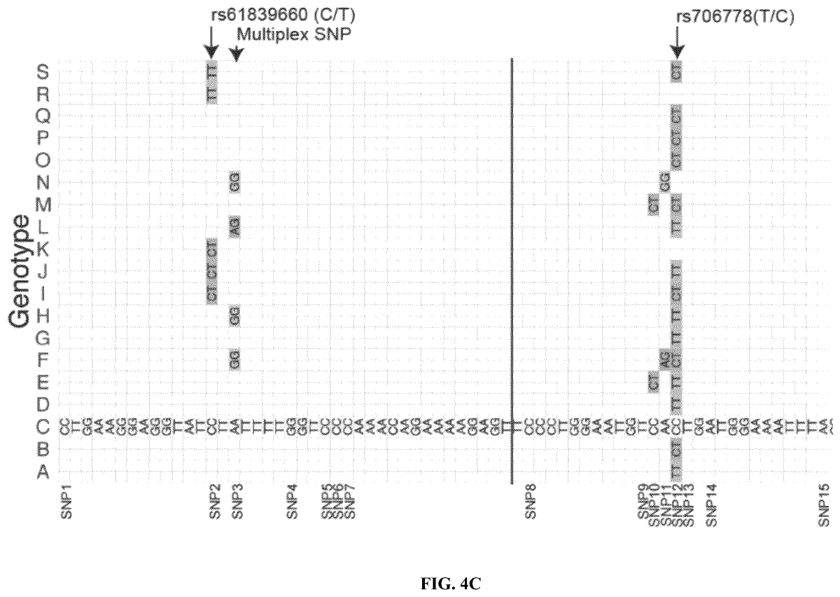

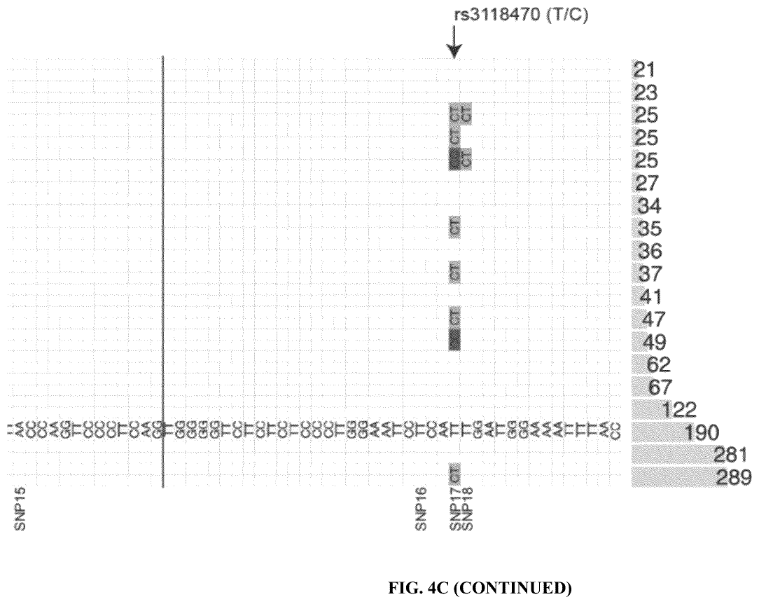

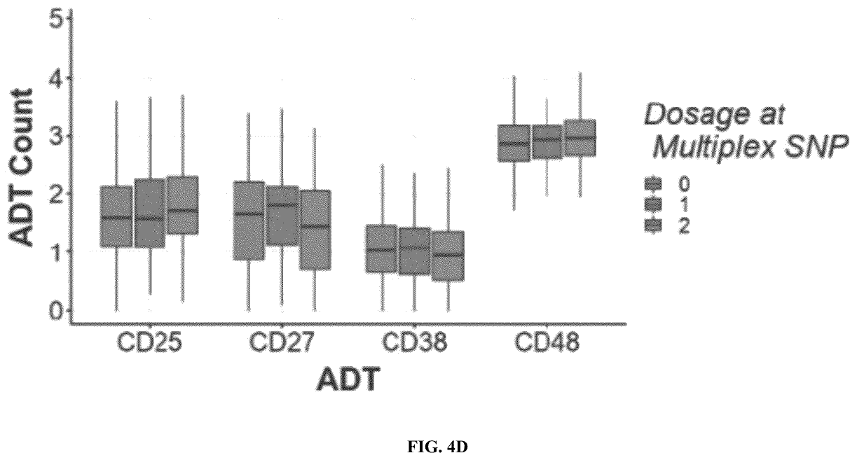

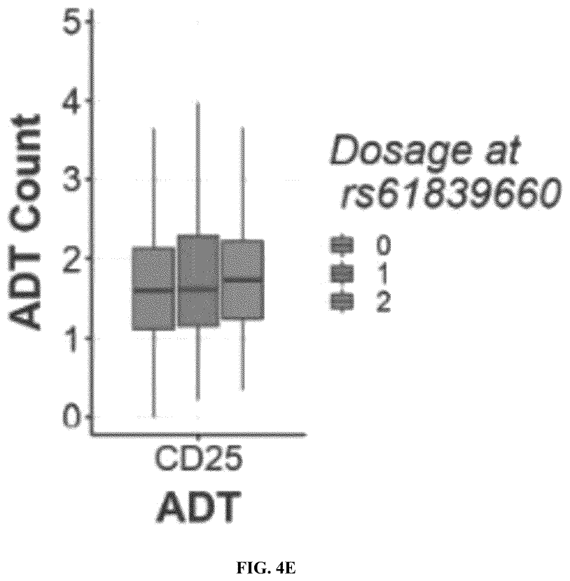

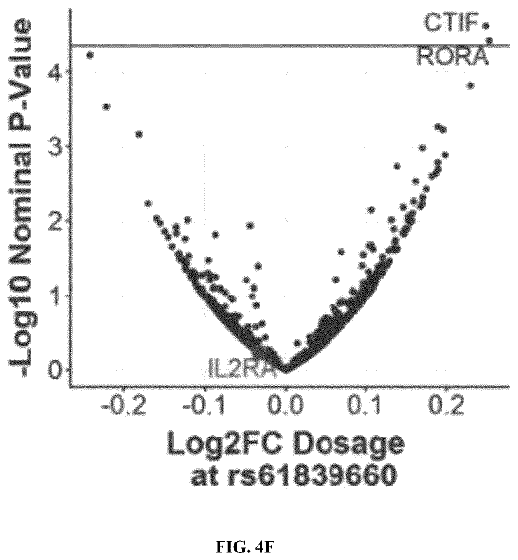

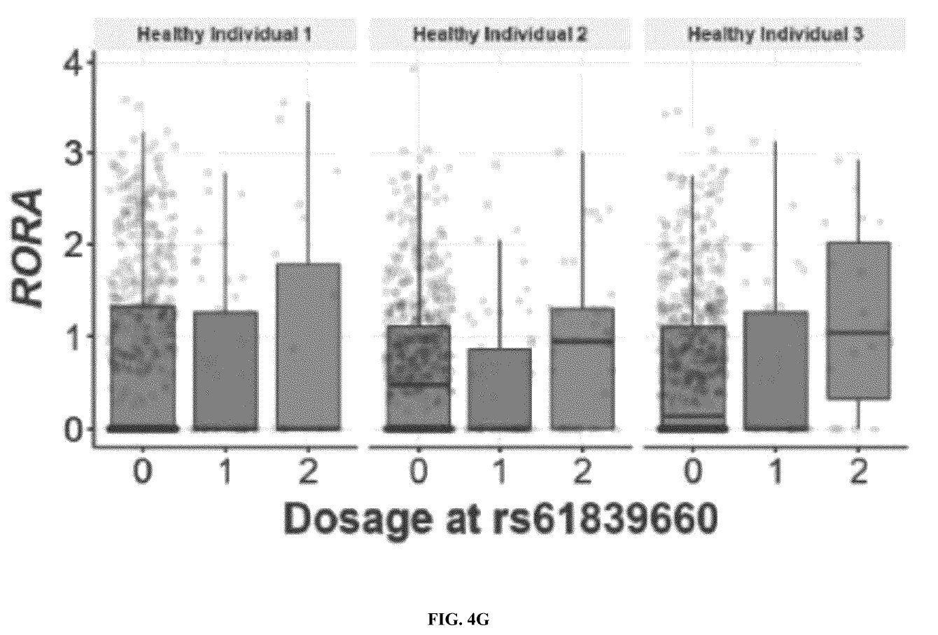

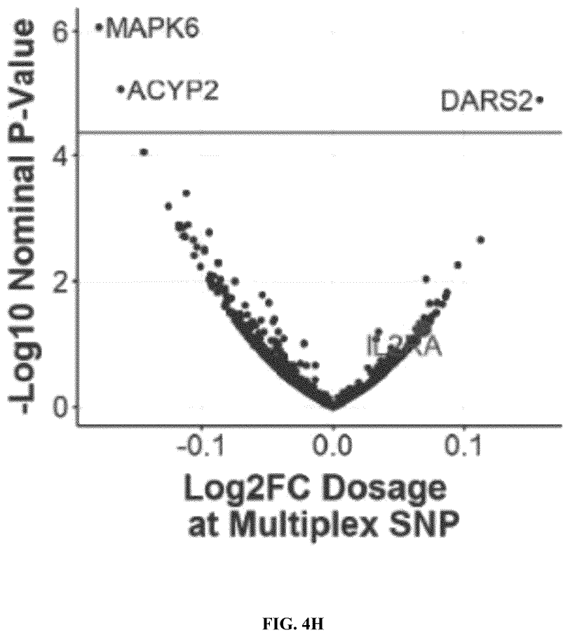

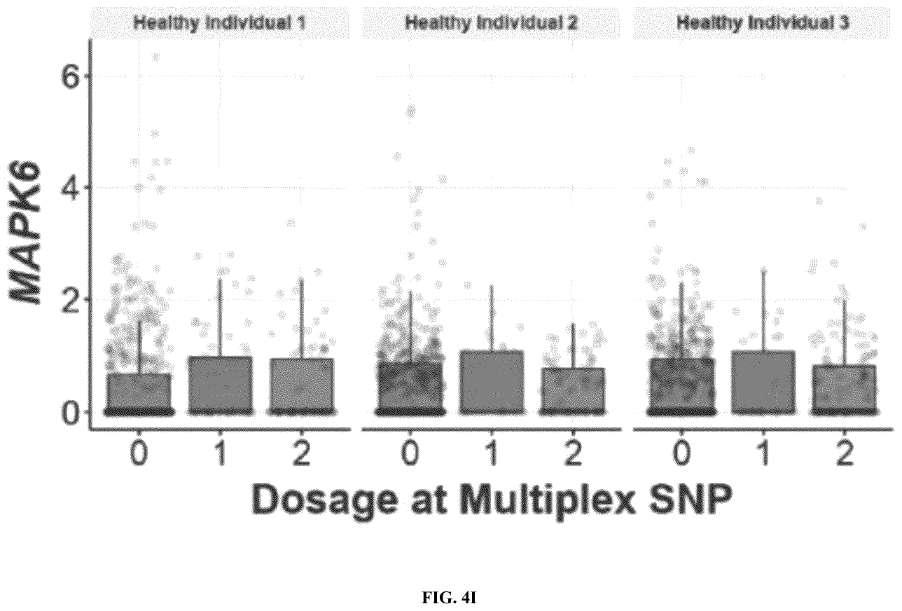

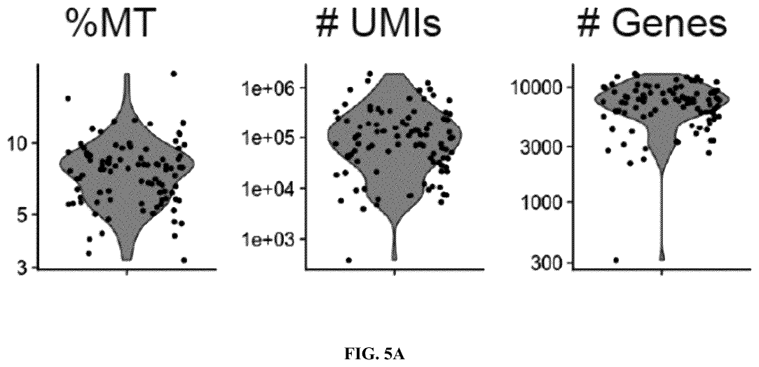

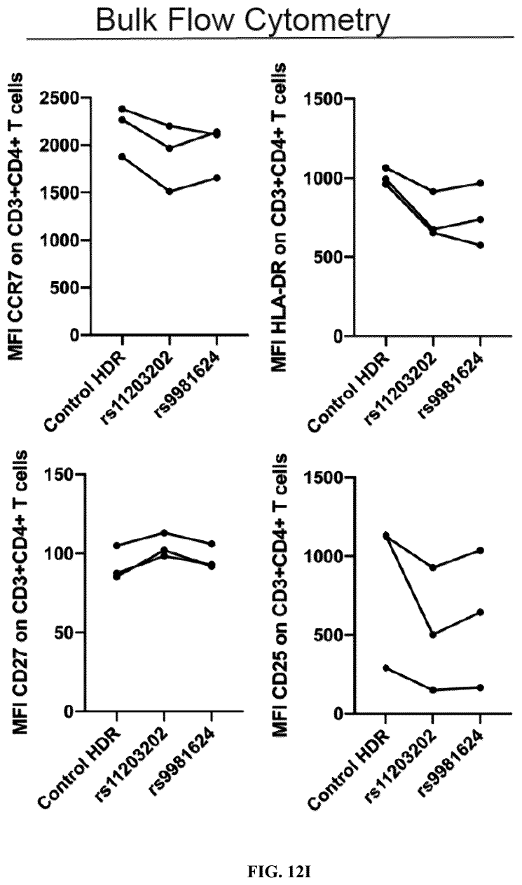

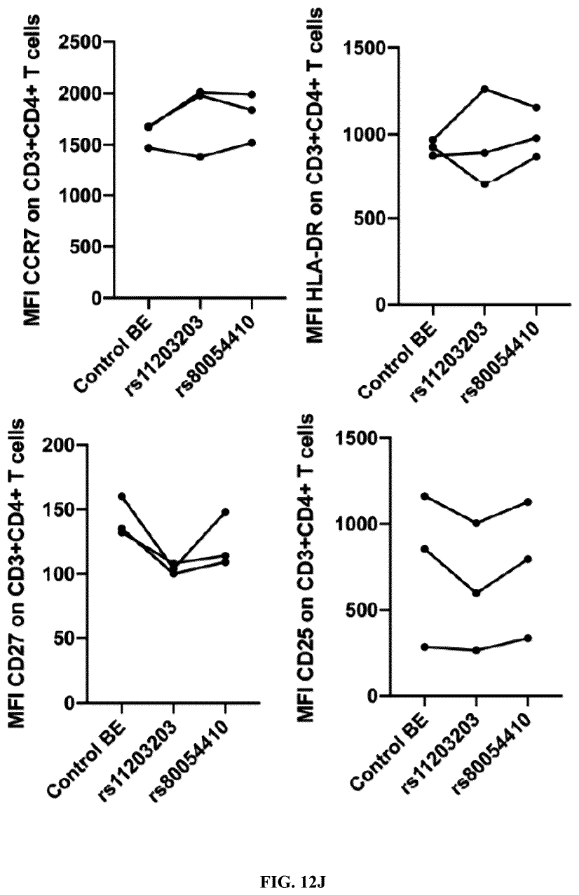

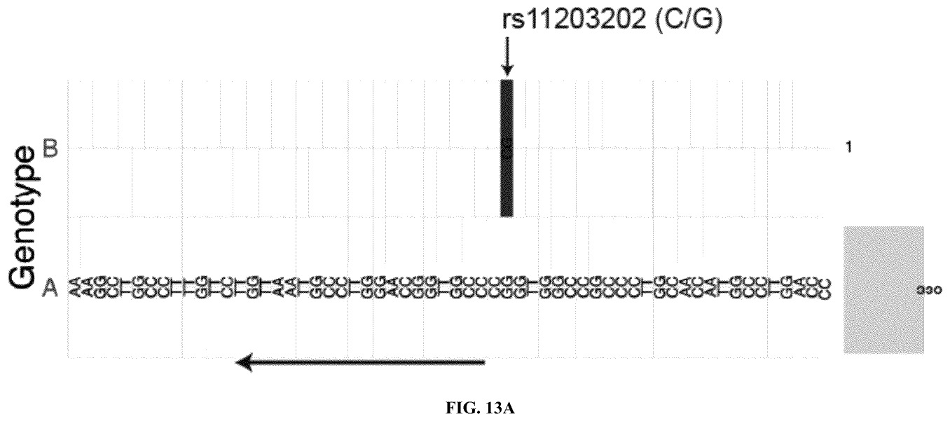

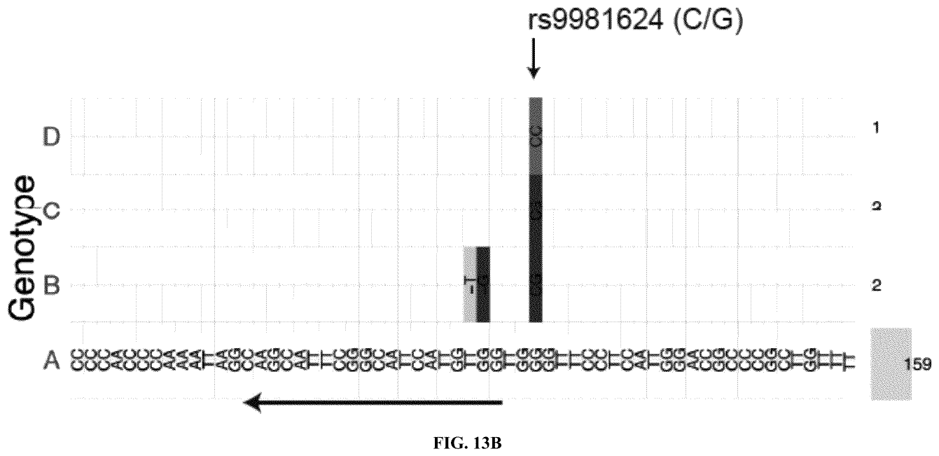

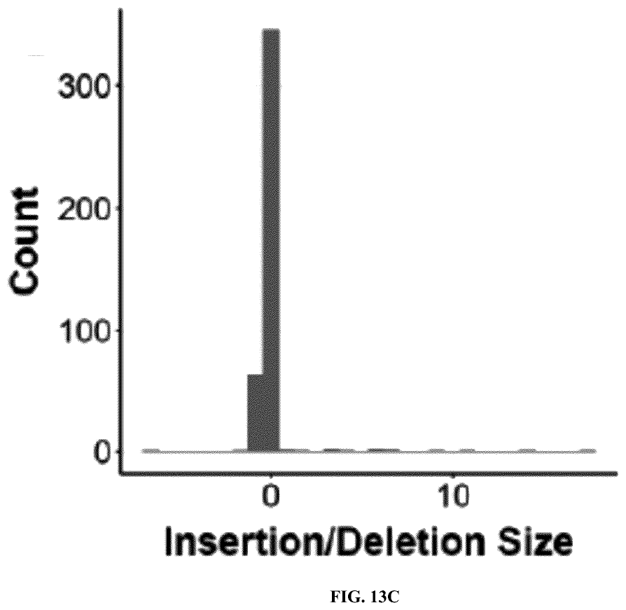

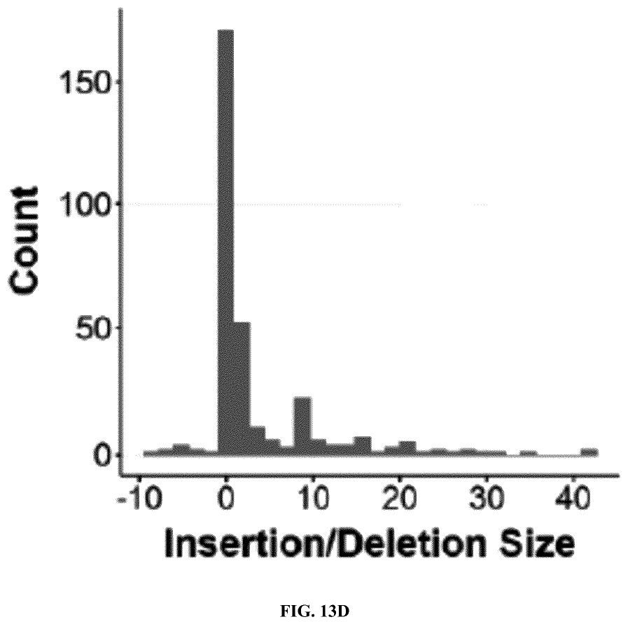

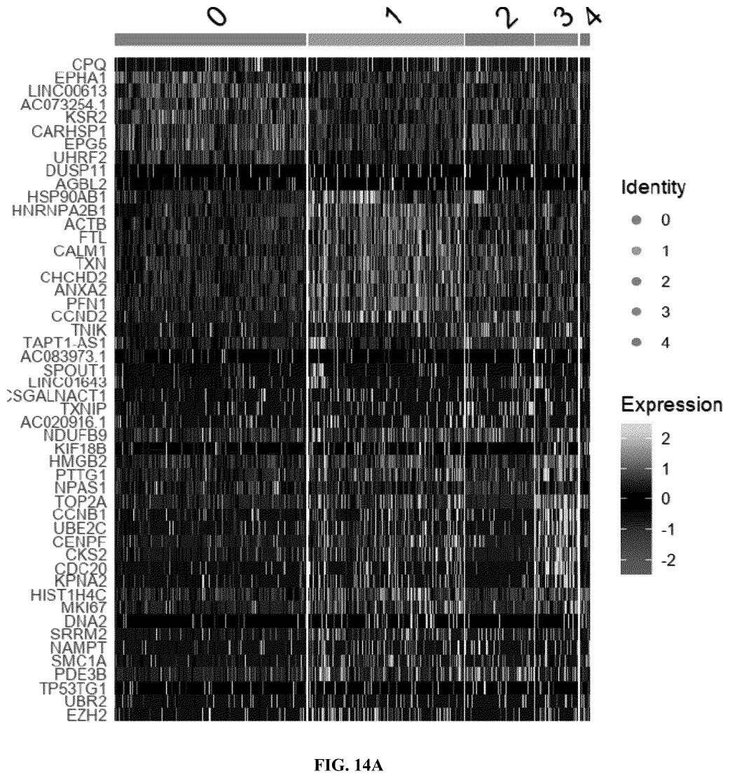

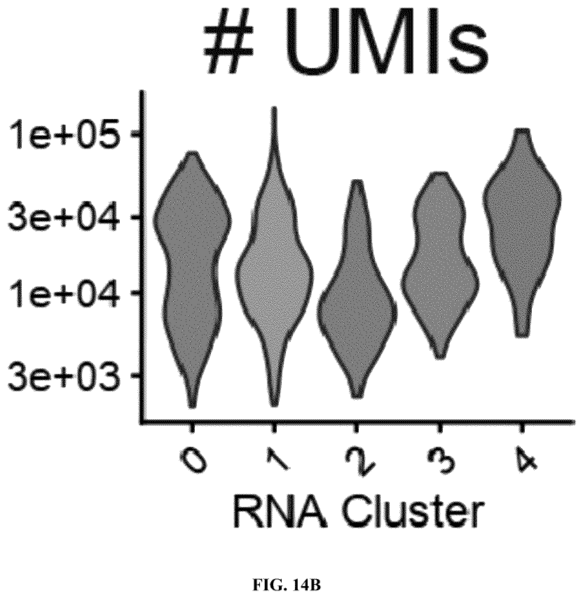

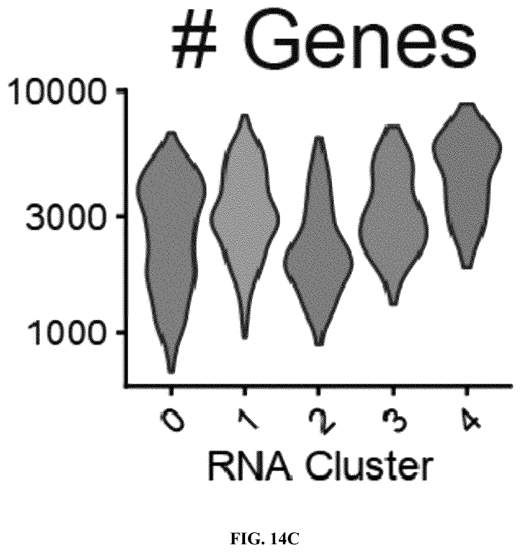

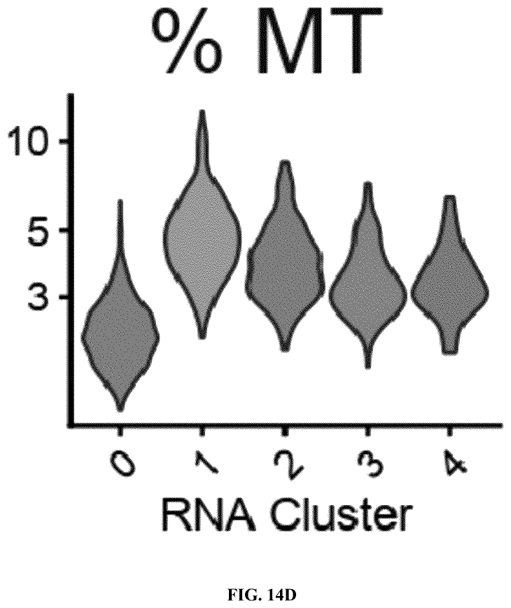

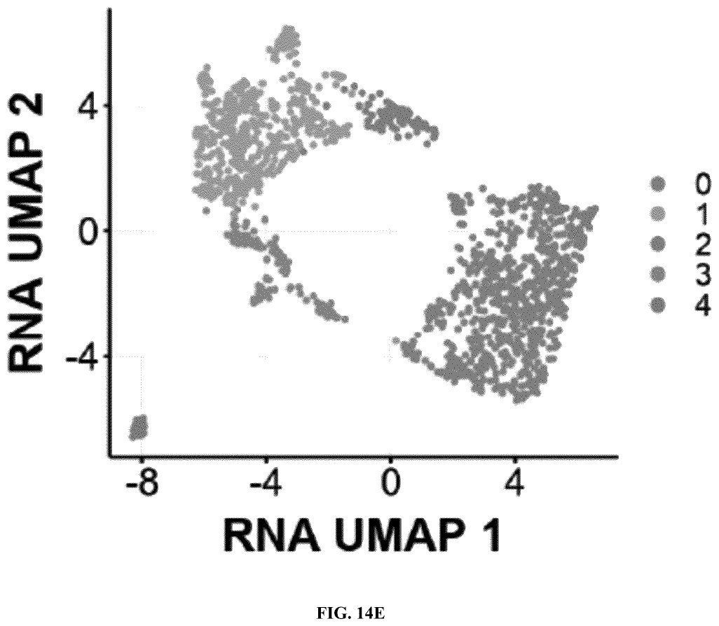

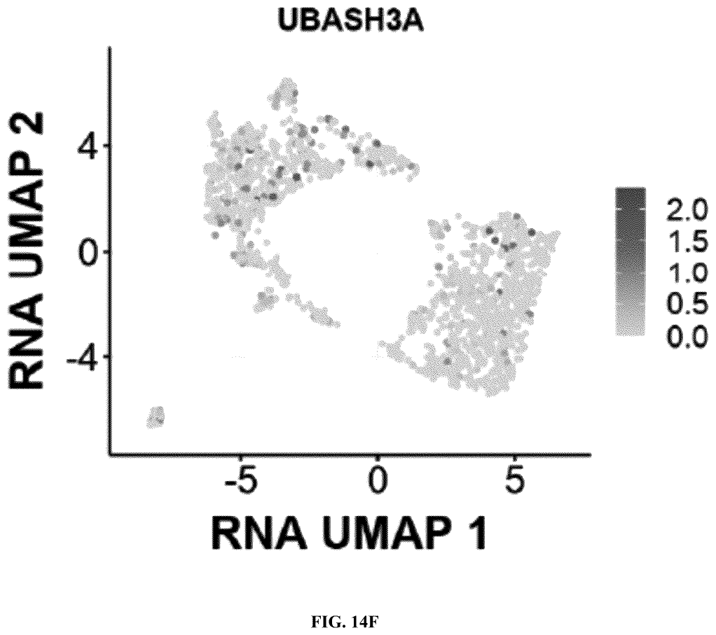

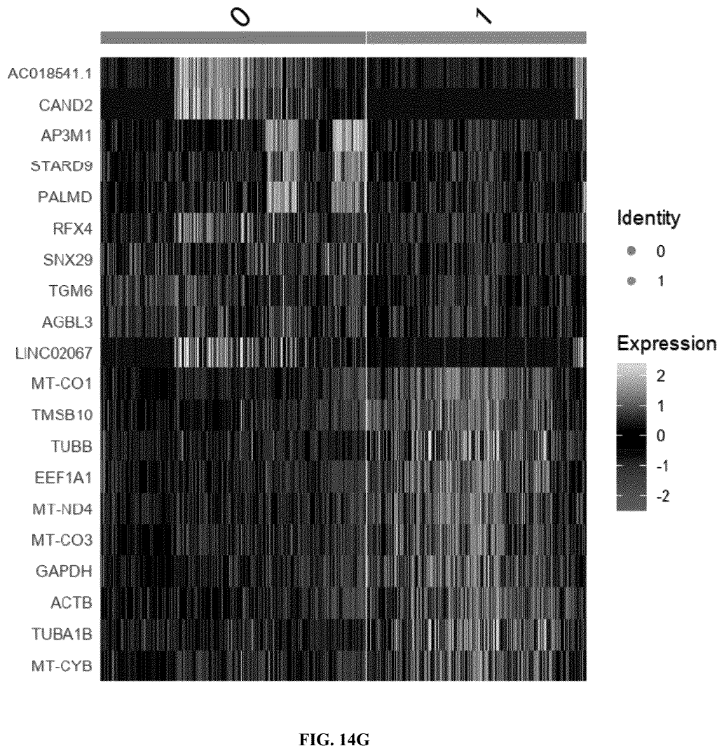

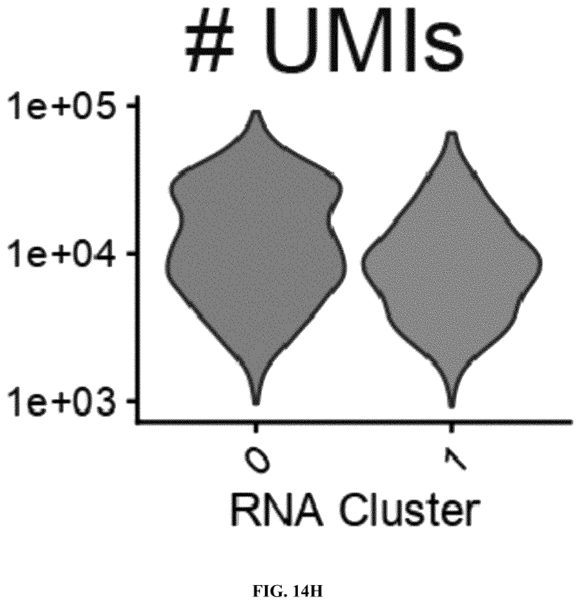

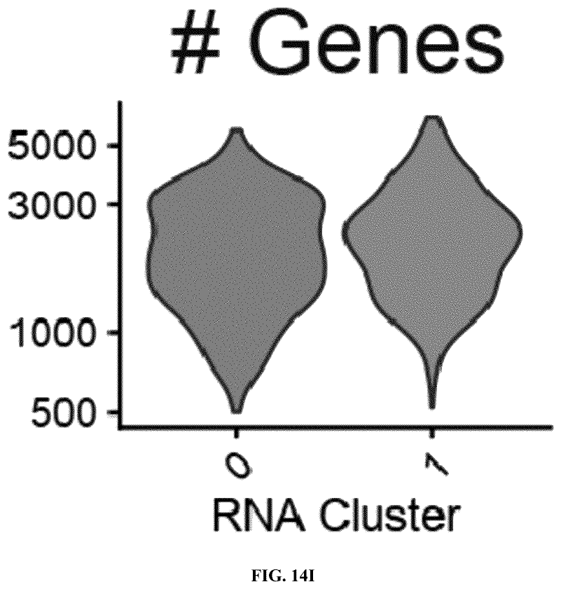

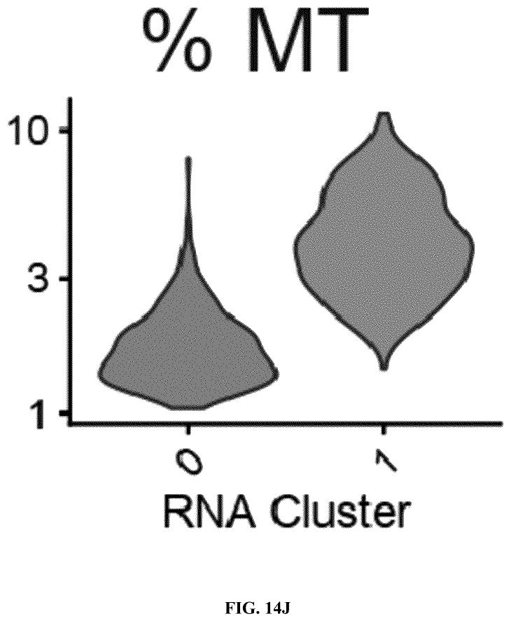

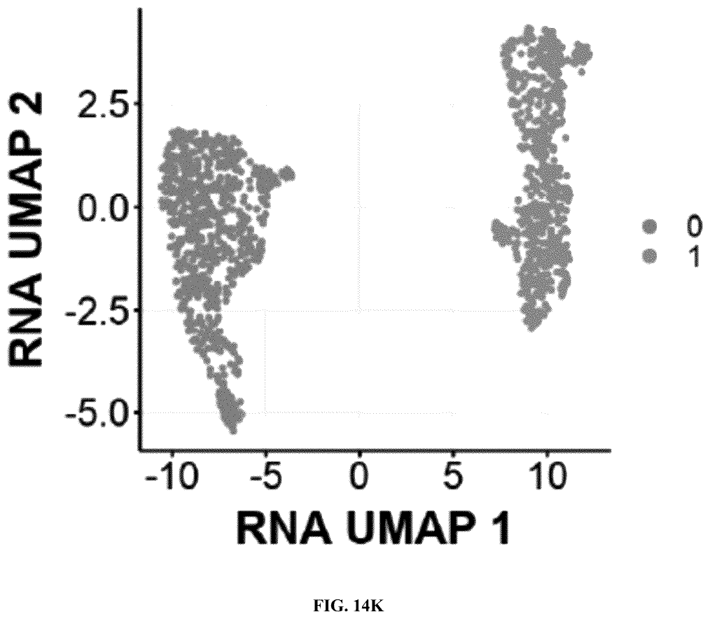

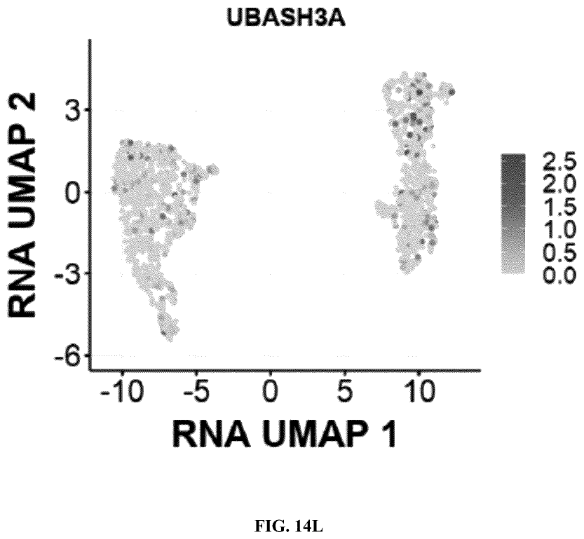

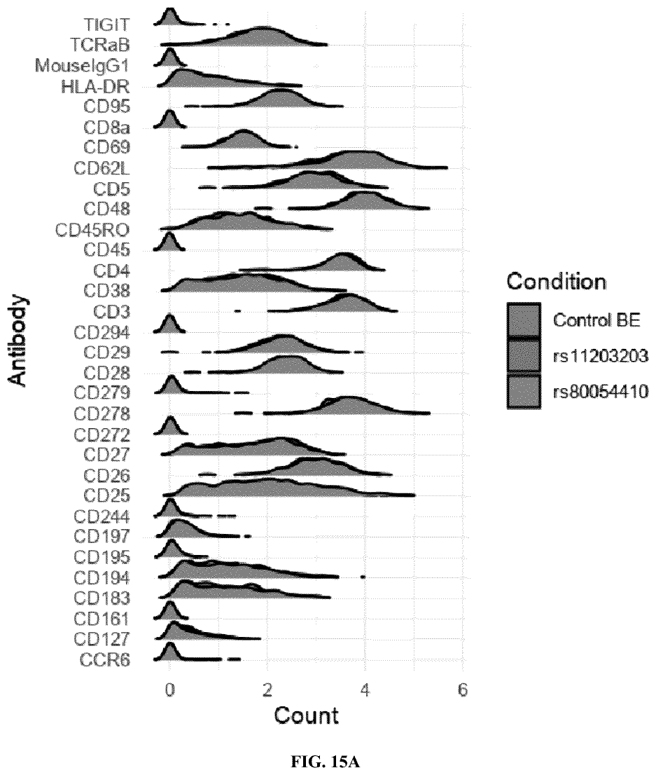

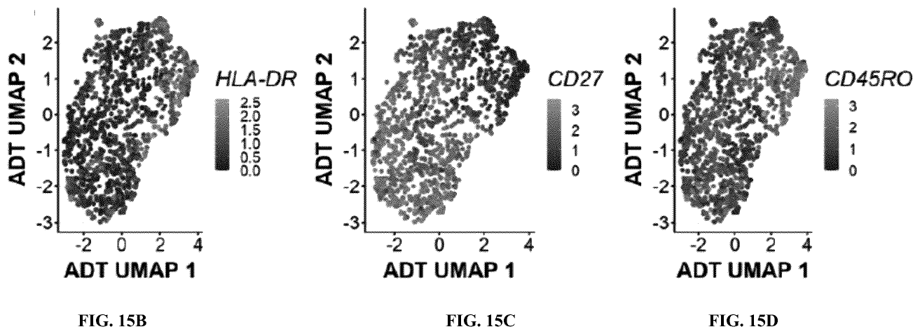

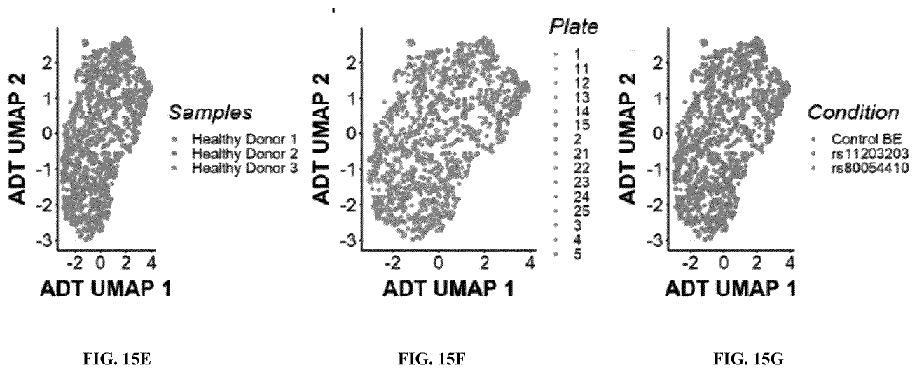

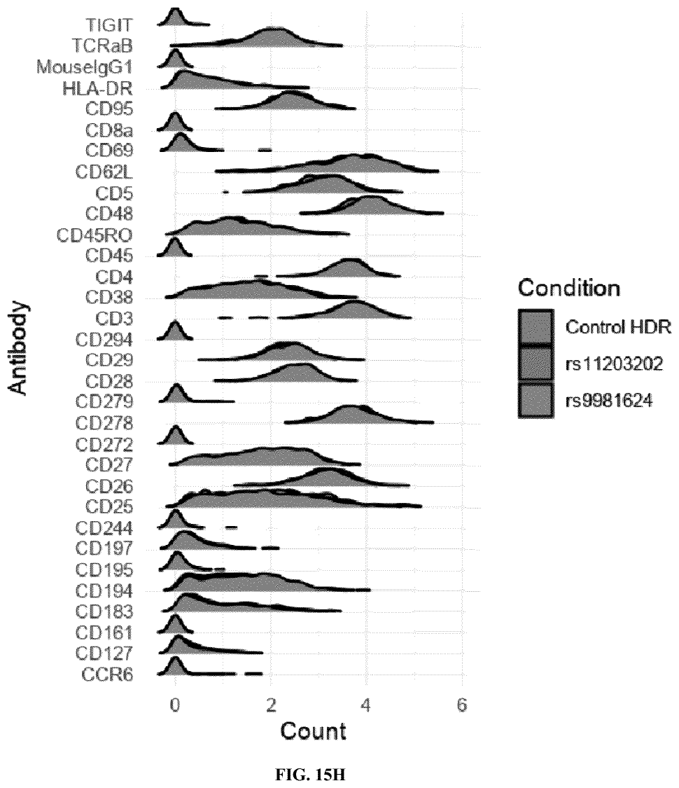

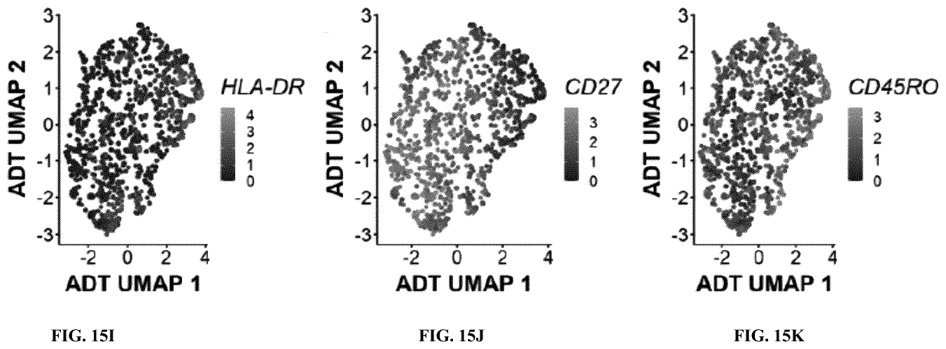

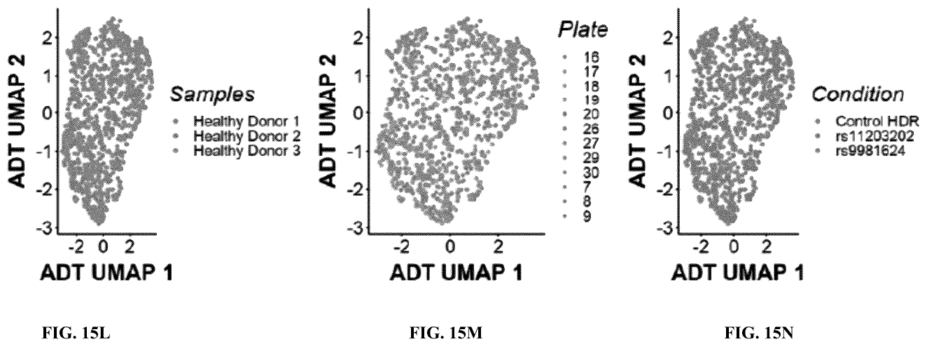

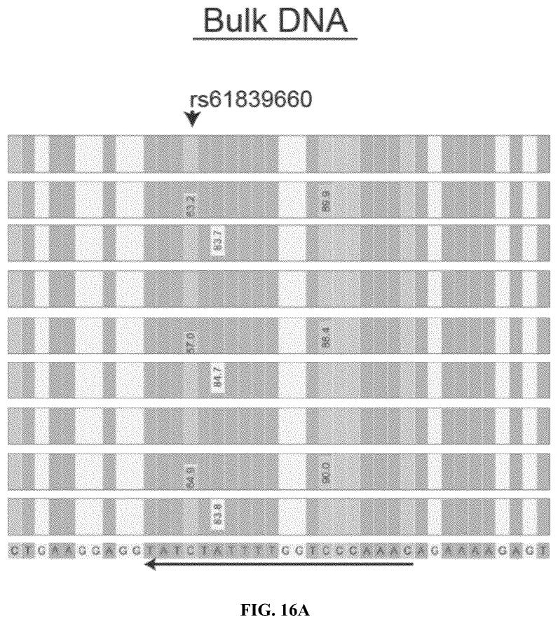

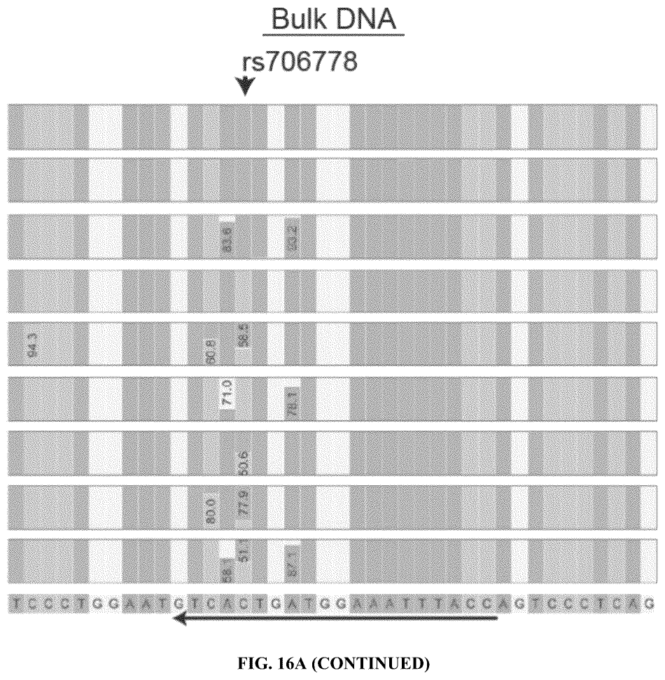

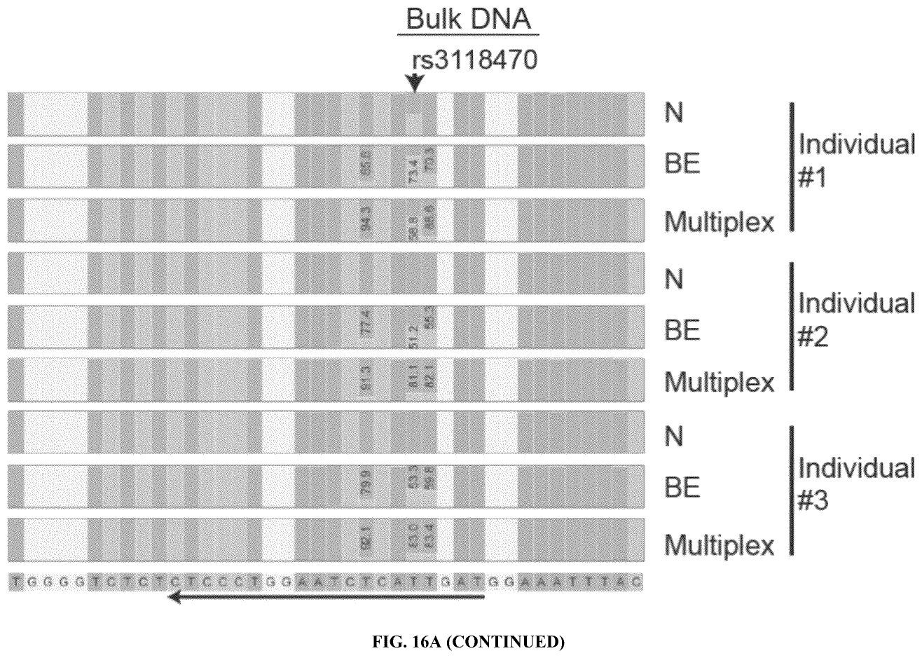

Single cell MINECRAFTseq provided a clearer picture of the editing effects in both base-edited and HDR-edited variants. Single-cell genomic DNA sequencing identified considerable bystander editing in base-edited cells and distinct clusters of indels in HDR edited cells (FIGS. 3B-3E). Although HDR editing was successful for both rs9981624 and rs11203202, it was incredibly rare with insertions dominating editing surrounding rs11203202 and deletions in rs9981624 (FIGS. 13A-13D). The advantages of the method allows for utilization of this varied and heterogeneous editing to still discern effects on gene and protein expression. Differential gene expression of base-edited cells modeling both the dosage of the targeted single-nucleotide polymorphism (SNP) along with plate revealed changes associated with rs11203203 in RIPK1 and NPAS1 expression (FIGS. 3F-3H). Similarly, for HDR edited samples editing around rs11203202 and not rs9981624 caused an upregulation of several genes including IL2RA (FIGS. 3I-3K) providing a possible link to proliferation and IL-2 signaling. There was no effect on mRNA clustering or ADT expression (FIGS. 14A-14L and 15A-15N). Overall, using this multi-omic, single-cell approach with a limited number of samples and cells it was discerned that two of the variants in UBASH3A were causal, potentially impacting cell proliferation and IL2 signaling. Lastly, single cell MINECRAFTseq was applied to the IL2RA locus in primary CD4 T cells. Previous computational work identified 3 possible causal variants of which two (rs706778 & rs3118470) had high TBET IMPACT scores and were chosen for functional follow up. Another previously validated variant, rs61839660, was also selected for investigation as it has been shown to have a strong activation dependent effect that regulates T cell differentiation. Three individuals were recruited with unique genotypes at the three variants of interest and used CRISPR-Cas base-editors to either target each variant individually or as one large, multiplexed pool (FIGS. 4A and 4B). A combination of base-editors and different genotypes were selected in order to investigate the effects on heterozygotes and non-targetable editing sites.

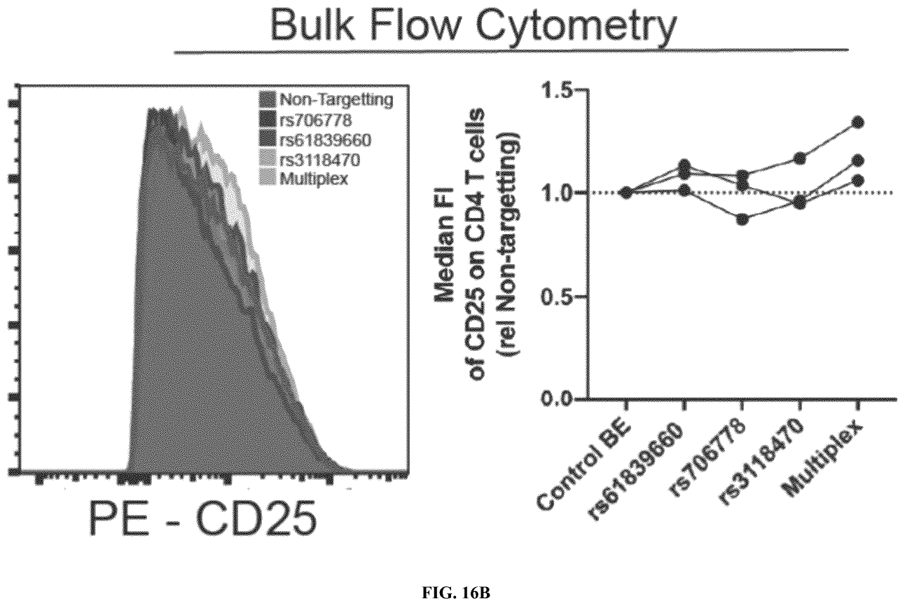

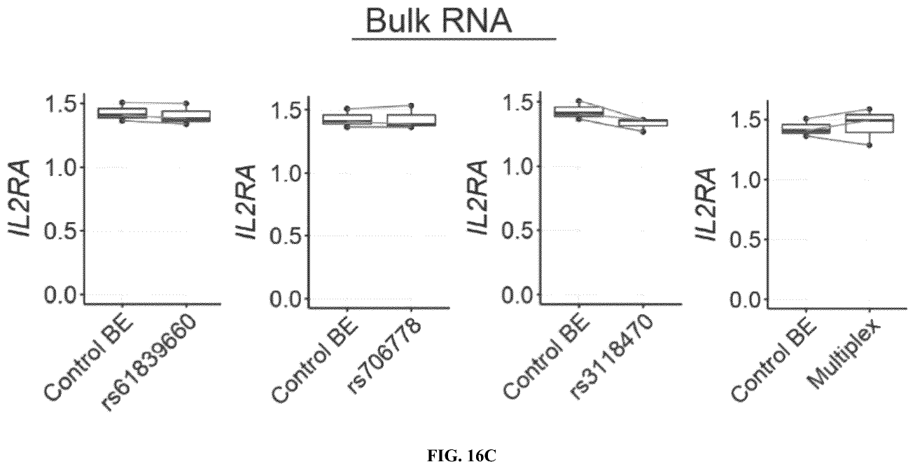

Bulk analysis of flow cytometry, genomic DNA, and mRNA revealed that multiplexed editing with different base-editors was possible but had preferential usage of A to T base-editors (FIGS. 16A-16C). Bulk flow cytometry data, but not bulk mRNA sequencing, showed a consistent increase in CD25 expression from rs61839660 and the multiplex samples (FIGS. 16A-16C).

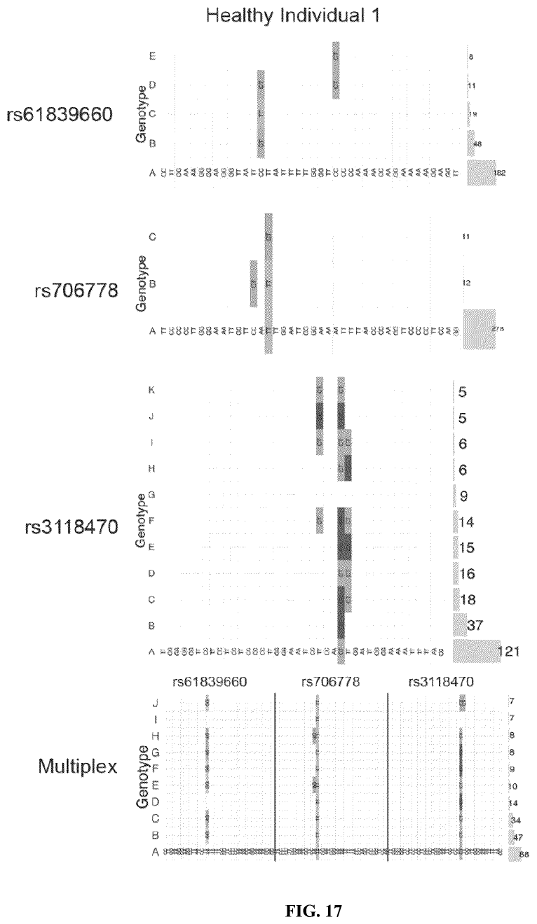

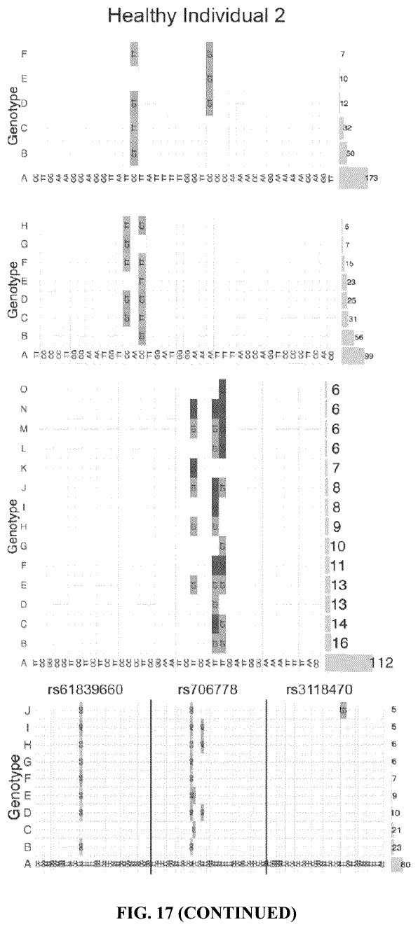

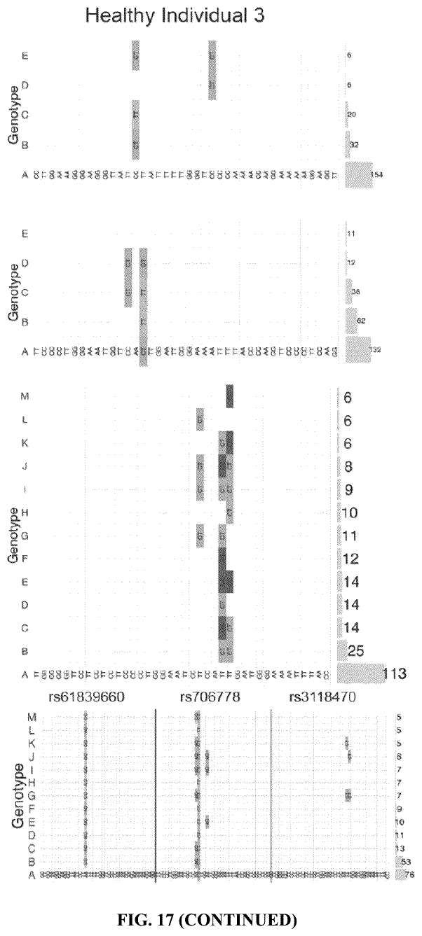

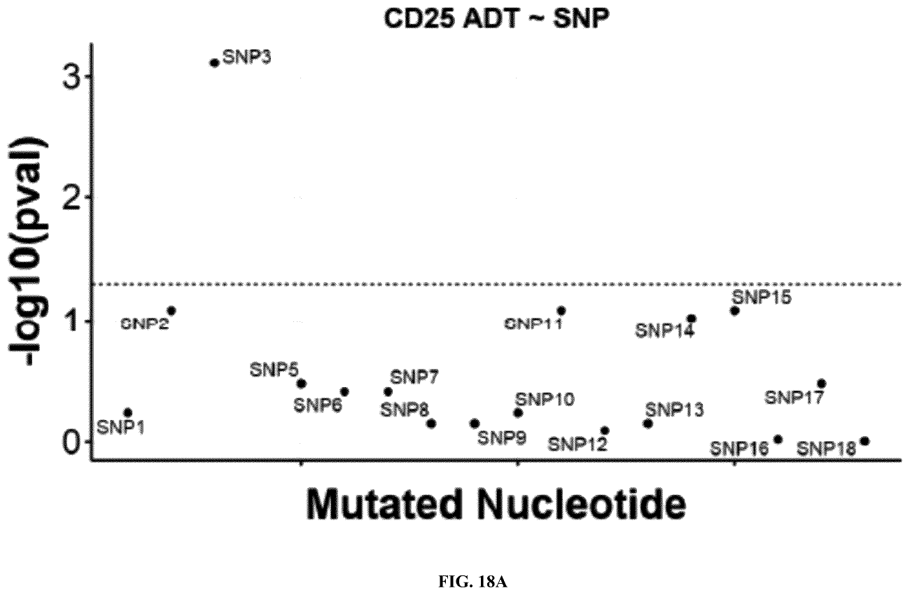

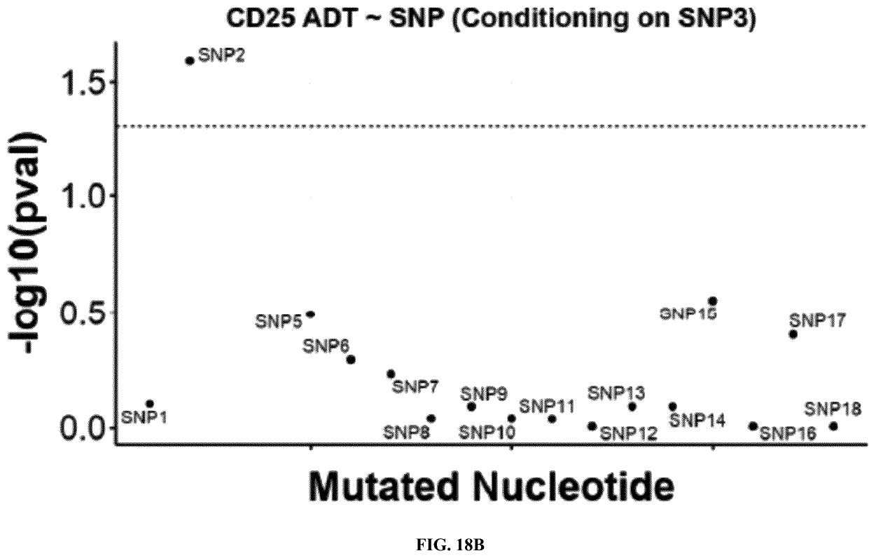

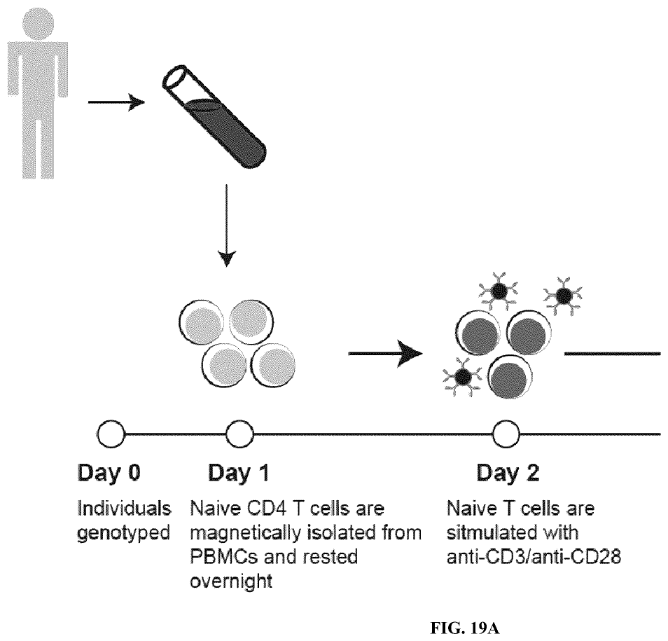

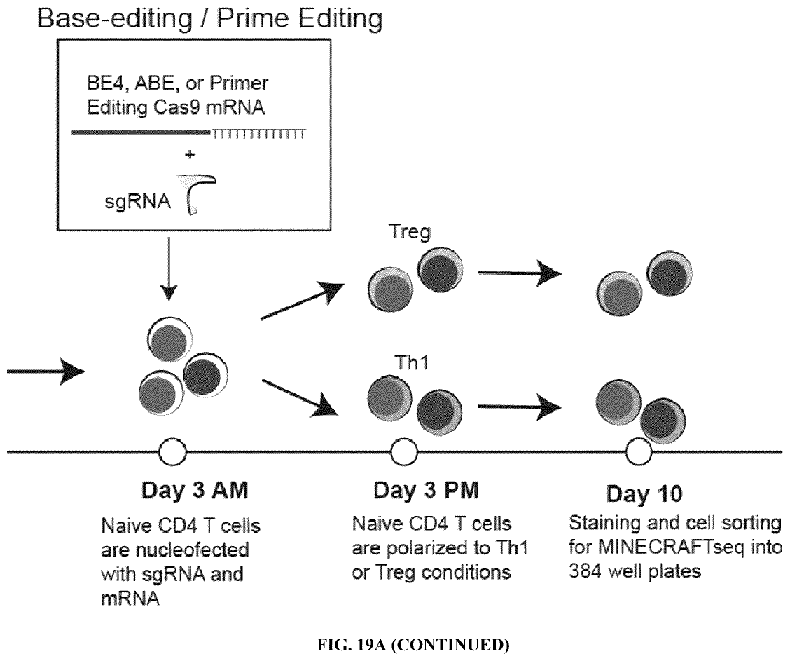

Single cell MINECRAFTseq identified many unique genotypes in various combinations (FIGS. 4C and 17). As expected, targeting heterozygous individuals could be used to convert to homozygotes. Given the wide range of induced mutations, every targeted nucleotide was codified in the regions of interest (labelled as SNP1-SNP18) (FIG. 4C). Using these labels, it was investigated which targeted nucleotide was correlated to CD25 ADT expression using a linear regression framework accounting for plate effects (FIGS. 18A and 18B). It was found that targeting SNP3 (hereafter named the multiplex SNP) and not any of the investigated variants had the strongest effect on CD25 expression. When this analysis was conditioned on SNP3, a secondary effect in rs61839660 was identified (FIGS. 18A and 18B). Using additional linear models it was determined that significant changes in CD25, CD27, CD38, and CD48 were associated with the multiplex SNP and only CD25 with rs61839660 when conditioned on the multiplex SNP (single nucleotide polymorphism) (FIGS. 4D and 4E). Using the integrated single cell genomic DNA and mRNA, differential gene expression was performed to identify a correlation to CTIF and RORA with dosage at rs61839660 (conditioning on the multiplex SNP), indicative of a possible link to Tregs (FIGS. 4F and 4G). Similarly, dosage at the multiplex SNP was correlated to MAPK6, ACYP2, and DARS2 gene expression (FIGS. 4H and 4I). Taken together, the single cell multi-omic approach was used to experimentally investigate three variants in primary CD4 T cells and identify a clear effect at rs61839660 and a nearby nucleotide, helping to further resolve this locus and its effects.

As the lists of loci and variants implicated in disease continue to grow, scalable and flexible methods need to be used to investigate potential causality. Herein is provided a highly customizable multi-omic approach applied to a multitude of genomic editing techniques in several disease loci in cell lines and primary immune cells to find causal variants, regions, and genes. By capturing and integrating four single cell modalities, index flow cytometry, genomic DNA amplicons, 3′ mRNA sequencing, and ADT sequencing, single cell MINECRAFTseq presents a holistic view of genomic editing paving the way for future studies linking variant to function in primary immune cells.

The following materials and methods were employed in the above examples.

### Cell Line Cultures and Genomic Editing

HH cutaneous T cell lines (ATCC: CRL-2105) and Jurkat E6-1 (ATCC: TIB-152) were cultured in complete RPMI, RPMI 1640 supplemented with 10% heat inactivated FBS, and 1% non-essential amino acids, sodium pyruvate, HEPES, L-Glutamine, Penn-Strep, and 0.1% b-mercaptoethanol. To investigate regulatory regions around HLADQB1, the nearest sgRNA to the validated single-nucleotide polymorphism (SNP) of interest was selected and HH cells genomically modified as previously described (CITATION, Nat Gen, Maria and Yuriy). Briefly, 40 μM Cas9 protein (QB3 Mircolabs) was mixed with equal volumes of 40 μM modified sgRNA (Synthego) and incubated at 37° C. for 15 minutes to form ribonuclear protein (RNP) complexes. HH cells were nucleofected with 24 of RNPs in an Amaxa 4D nucleofector (SE protocol: CL-120). Cells were immediately transferred to 24 well plates with pre-warmed media and cultured. After 10 days, cells were single cell sorted with BD FACS ARIA II into 96 well plates for processing following a modified TARGETseq protocol. For Jurkat cells targeting single-nucleotide polymorphism (SNP) rs61839660, 1 μl of mRNA (2 ug/ul) encoding the base editor BE4-NG was mixed with 1 μl of 40 μM modified sgRNA (Synthego) targeting the variant of interest. Jurkat cells were then nucleofected with 2111 of mRNA/sgRNA mixture in an Amaxa 4D nucleofector (SE protocol:CL-120). Cells were incubated as described above in 24 well plates for 7 days then stimulated for 18 hours with anti-CD3/anti-CD28 microbeads (ThermoFisher) at a ratio of 1 bead to 1 cell. After stimulation, Jurkats were stained with ADT antibodies, single cell sorted, and processed with one of four optimization protocols. ADT staining of Jurkats was performed identical to staining of primary CD4 T cells described below.

### Healthy Individual Recruitment, PBMC Isolation and CD4 T Cell Magnetic Selection

To investigate select variants using single cell MINECRAFTseq protocols, healthy individuals were recruited and 40-50 ml of peripheral blood was processed under an IRB-approved protocol (IRB #2008P000427). PBMCs were isolated by layering Ficoll Paque (Sigma-Aldrich) underneath 1:1 PBS-diluted blood followed by centrifugation. Buffy coat layers were extracted and washed in PBS and then resuspending in XVIVO15 Media(Lonza) supplemented with 5% FBS (Gemini Bio), 55 μM 2-mercaptoethanol (Sigma), and 10 mM N-acetyl-L cysteine (Sigma), hereafter referred to as cVIVO15. At this stage, 500K cells were taken for DNA isolation and Sanger Sequencing of selected variants. The remainder of the cells were stored by adding an equal volume of freezing media (10% DMSO and 50% FBS in xVIVO15) and frozen in liquid nitrogen until use. All individuals were between 20 to 40 years old with no reported autoimmune disease. For isolation of CD4 T cells, frozen PBMCs were quickly thawed and put immediately in warm cXVIVO15. Cells were washed twice and the total CD4 T cell isolated using a magnetic negative selection kit (Miltenyi, CD4⁺ T cell Isolation Kit human) following manufacturers protocols.

Isolated cells were then rested overnight at a concentration of 2.5 million/250 μl of cXVIVO15 mL in 96 well U bottom plates until use.

### Sanger Sequencing of Selected Variants

Prior to performing CRISPR editing, the genotype of the individuals at targeted variants was identified by Sanger sequencing. Genomic DNA was isolated using a Qiagen DNA extraction kit following manufacturers protocols. A 200 bp-1 kb fragment surrounding the variants of interest was then amplified using custom PCR primers and Sanger sequenced (Eurofins Genomics). Chromatograms of the sequences were analyzed with SNAPGENE (v4.3.6) and genotypes determined based on distributions at the variant of interest.

### CRISPR Editing of Primary CD4 T Cells

After resting isolated CD4 T cells overnight, 1 million cells are stimulated with anti-CD3 and anti-CD28 dynabeads (ThermoFisher) at a ratio of 1:1 (cell:dynabead) in the presence of 5 ng/ml rhIL-2 (Biolegend) in 48 well plates. After 2 days, cells are extracted and dynabeads removed using a magnet before proceeding to CRISPR editing. To study regions and variants of interest, CRISPR-Cas9 C to T(BE4-NG), A to G base-editors (ABE8e-NG), or CRISPR-Cas9 mediated HDR repair was used. For base-editors, 0.5 million stimulated CD4 T cells were nucleofected with 1111 of mRNA (2 ug/μl) encoding the modified Cas9 protein complexed with 1 μl of sgRNA (40 μM, Synthego) in an Amaxa 4D nucleofector (P3 protocol:EH-115). To induce HDR repair, 0.5 million CD4 T cells were nucleofected with 2111 of Cas9 RNPs and 1111 of asymmetrical ssDNA donors in an Amaxa 4D nucleofector (P3 protocol:EH-115). Following nucleofection, cells were transferred to 48 well plates and cultured in cXVIVO15 media supplemented with 5 ng/ml rhIL-2 until use.

Cell Staining with Indexing and Oligo-Conjugated Antibodies.

After culturing nucleofected samples for an additional 7 days, cells were isolated, washed, and resuspended in FACS buffer (2% FBS in PBS with EDTA). An aliquot of cells was then used for bulk RNA, DNA, and flow cytometry analysis. The remainder of the sample (200K cells) was stained with fluorophore-conjugated antibodies for 20 minutes on ice. After staining, cells were washed, counted, and different conditions pooled together on a per sample basis. Samples were then spun down and stained with oligo-conjugated antibodies as previously described. Briefly, 1 million cells were resuspended in FC blocking solution (Biolegend) for 5 minutes at room temperature followed by staining with room temperature antibodies for an additional 15 minutes. Afterwards, samples were stained with the cold-temperature antibody mix for 20 minutes on ice before washing and proceeding to single cell sorting.

### Bulk RNA and DNA Isolation

Cells were pelleted, resuspended in RLT+ buffer (Qiagen), flash frozen on dry ice and stored at −80 until processing. For RNA/DNA isolation, samples were thawed, vortexed and incubated for 5 minutes at room temperature before proceeding to RNA/DNA isolation using the Qiagen RNA/DNA extraction kit following manufacturer protocols. After isolation, RNA and DNA concentrations were measured by spectrometry (Nanovue) and stored at −20 until use. For bulk mRNA sequencing, samples were converted to cDNA with custom oligoDT and TSO primers in an optimized Reverse Transcription reaction and then amplified by PCR for 10 cycles. Full length cDNA was then tagmented with Nextera XT reagents and custom Illumina adaptors added to amplify 3′mRNA products for sequencing. For DNA, successive nested PCR reactions were used to amplify 200-400 bp genomic regions of interest with custom Illumina adapters added for sequencing. Bulk samples were sequenced alongside single cell libraries.

### Bulk Flow Cytometry

Stimulated and genomically edited CD4 T cells were assayed for expression of key protein markers by flow cytometry on day 7 post-nucleofection with a panel of fluorophore-conjugated antibodies. For all samples, cells were isolated, washed twice in PBS, and FC receptors blocked with FcX True Stain (Biolegend) for 15 minutes on ice followed by staining with directly-conjugated antibodies for 30 minutes on ice. Cells were then washed and samples analyzed on a BD LSR Fortessa. All data was processed using FlowJo and analyzed with GraphPad PRISM.

Modified TARGETseq—Genomic DNA and mRNA Sequencing from HH Cells.

Genomically editing HH cells were processed with a modified TARGETseq approach that allowed for the capture of genomic DNA amplicons and mRNA with increased multiplexing. Cells were edited as described above, washed twice, filtered through 40 μM, and single cell sorted into 96 well plates with lysis buffer and well/cell barcoded oligoDT primers using a FACS ARIA II. After sorting, plates were spun down and incubated for 5 minutes at room temperature before being flash frozen on dry ice and stored at −80 until use. After thawing, plates were incubated at 72 degrees for proteinase deactivation and cDNA synthesis performed. After synthesis, cDNA was amplified in the presence of genomic DNA specific primers targeting the targeted HLADQB1 region with SeqAMP PCR reagents for 22 cycles. After amplification, 1 μl of the product was taken for further amplification of genomic DNA with nested HLADQB1 primers with well/cell specific barcodes. The remainder of the cDNA amplified product was solid phase reversible immobilization (SPRI) cleaned at 0.65× (beads:sample) to purify the full length cDNA and then tagmented with Nextera XT reagents and custom Illumina adaptors added to amplify 3′mRNA products for sequencing. After the nested genomic DNA PCR, samples were pooled per column (8 reactions) and further amplified with custom Illumina primers adding indexing barcodes for sequencing. 3′ mRNA and DNA libraries were SPRI cleaned at 1×, concentrations measured by QuBit (ThermoFisher) using a 1×HS DNA kit, distributions examined on a D1000 Agilent TScreenTape, and submitted to sequencing at either the Genomic Platform at the Broad Institute or the Molecular Biology Core Facilities (MBCF) at Dana-Farber Cancer institute (DFCI).

### Optimizations

Genomically editing Jurkat cells were processed using four different protocols. As before, cells were edited, stained with oligo and fluorophore conjugated antibodies sorted into PCR plates with a lysis buffer, and stored until use. For library generation, plates were incubated at 72 degrees Celsius for proteinase deactivation and cDNA synthesis was performed. After synthesis, cDNA was amplified in the presence of gDNA specific primers targeting the IL2RA region with one of four reaction conditions for 20 cycles. After amplification, an aliquot of product was taken for further amplification of genomic DNA with nested IL2RA primers with well/cell specific barcodes. The remainder of the product was solid phase reversible immobilization (SPRI) cleaned for size selection at 0.65× (beads:sample) to purify the full length cDNA. The flowthrough was collected and re-SPRIed at 2× to isolate the ADT fraction. Full length cDNA was then tagmented as before and amplified with custom Illumina adaptors for sequencing. The ADT fractions were PCR amplified with custom Illumina adaptors for 10 cycles. As before, library concentrations and distributions were measured before proceeding to sequencing.

### MINECRAFTseq

Genomically editing primary CD4 T cells were stained with oligo and fluorophore conjugated antibodies and sorted into PCR plates with 2.1 μl of lysis buffer. Plates were stored at −80 degrees Celsius until use. For library generation, plates were thawed and incubated at 72 degrees Celsius for proteinase deactivation. An additional 2.91 μl of cDNA synthesis mixture was then added to each well containing MaximaH RT enzyme and a custom buffer with GTP and PEG (full details in Supplementary Tables). After first strand synthesis, an additional 7.5111 of PCR mix was added to amplify the cDNA, ADT, and genomic DNA. Specific genomic DNA primers targeting the variant of interest were added. After 20 cycles of amplification, 0.5111 of product was taken for further amplification of genomic DNA with nested primers containing well/cell specific barcodes. After nested genomic DNA barcoded, samples were pooled per plate, purified with 1×SPRI and DNA quantified with a QuBit. 5 ng of the product per plate was then amplified with custom Illumina compatible primers and cleaned with 1×SPRI before submitting to sequencing. The remainder of the cDNA product was pooled per plate and SPRI cleaned for size selection at 0.65× to purify the full length cDNA. The flowthrough was collected and re-SPRIed at 2× to isolate the ADT fraction. cDNA concentrations were measured on a QuBit and 0.5 ng used for tagmentation with the NexteraXT kit (Illumina). Following tagmentation, the 3′ end of the cDNA molecule was amplified with custom Illumina compatible primers. Following amplification, PCR products were cleaned with 1×SPRI reagents before being submitted to sequencing. The ADT fraction was quantified on a Qubit and 5 ng of product used for subsequence amplification with custom Illumina primers. Again, the final ADT product was purified with 1×SPRI and quantified before sequencing. For experiments involving multiple conditions per healthy individual, all related conditions were indexed with fluorophore conjugated antibodies and pooled into one sample prior to sorting. Each sorted and processed plate represents a mixture of conditions, reducing batch effects. For UBASH3A experiments, HDR and BE conditions were separately pooled and processed. For IL2RA experiments, all conditions were pooled and processed together. During genomic DNA amplification, all regions in the pool were amplified in the same reaction using multiple specific and nested primer sets.

### Illumina Deep Sequencing

Prior to submission to sequencing at the Genomics Platform at the Broad or the Molecular Biology Core Facilities (MBCF) at Dana-Farber Cancer institute (DFCI), sample DNA concentrations were quantified on a QuBit and distributions measured using a TapeStation (Agilent). All DNA amplicon libraries were sequenced on a MiSeq for 300 cycles. ADT amplicon libraries were either sequenced on a MiSeq for 50 cycles or pooled and sequenced with RNA libraries on a NextSeq550 for 75 cycles or NovaSeq6000 for 100 cycles.

### Sequencing Data Processing and Statistical Analysis

17 and IS demultipled raw fastqs were merged across different lanes and runs using a custom bash script. For analysis of DNA amplicon data, fastqs were further demultiplexed based on cellular barcodes using a custom bash script. Fastqs were then aligned to reference sequences with CRISPResso2. Deletion statistics, allele usage, and nucleotide modification frequencies were calculated and imported from CRISPResso2 output into custom R analyses and graphing pipelines. For all DNA analyses, cells were filtered on a minimum of 10 aligned reads. In order to identify cellular genotypes, expressed alleles were filtered on at least 10 reads and at least 10% of all recovered alleles. Assuming dizygosity, cells with more than 2 alleles after filtering were excluded from the analysis. At this stage, if only one allele was recovered, the cell was assumed to be homozygous. Heatmaps of DNA editing were generated from nucleotide modification tables generated with CRISPResso2. All nucleotide modification frequency (including substitution, deletions, and insertion) per nucleotide was used to generate heatmaps using the complex Heatmap package. Frequencies were binned into 3 groups, <0.3, 0.3-0.7, and >0.7 encompassing reference (0), heterozygote (0.5), and homozygote (1) editing for visualization. In these analyses, insertions were quantified as affecting both nucleotides at the insertion site (FIGS. 3B-3E). Clustering of DNA editing was performed with supervised k-means clustering. For analysis of ADT, kallisto KITE was used. References were created based on the barcodes used per experiment and ADT sequences aligned. UMI counts were generated, imported into R, and CLR normalized using a custom R function. For visualization, a PCA was performed on CLR normalized and scaled variable ADTs followed by plate correction using Harmony and Uniform Manifold Approximation and Projection (UMAP) dimension-reduction on harmonized PCs. Linear modeling of ADTs was performed in R with the lm function and significance calculated using an anova to the null model. For analysis of RNA, STARSolo was utilized to generate gene counts. A reference was created from the human GRCh38 transcriptome and reads mapped with custom barcode and UMI lengths. Resulting count matrices were imported into R and processed with Seurat. For all experiments, cells were filtered on at least 300 genes, 500 UMIs, and less than 10% mitochondrial reads. For visualization, PCA was performed on variable genes followed by batch correction with Harmony and dimension reduction with UMAP. Differential gene expression was performed on expressed genes (>30% of cells with non-zero expression) with DESeq2 modeling plate effects. Normalized and scaled counts were used for visualization. In some experiments, we noted that certain cellular barcodes resulted in inaccurate RNA mapping. For UBASH3A experiments, all cells with the cellular barcode CTGGTTCTGTTG (SEQ ID NO: 1) were excluded. For IL2RA experiments, all cells with the cellular barcode GCTACTCCAGTT (SEQ ID NO: 2) were excluded. For index flow cytometry analysis, raw fluorescence values were imported and processed in R using the ggcyto package. Using index flow cytometry information we also excluded any rare cells from the control non-targeting condition that had a non-reference genotype as quantified above.

### Protocol Modification

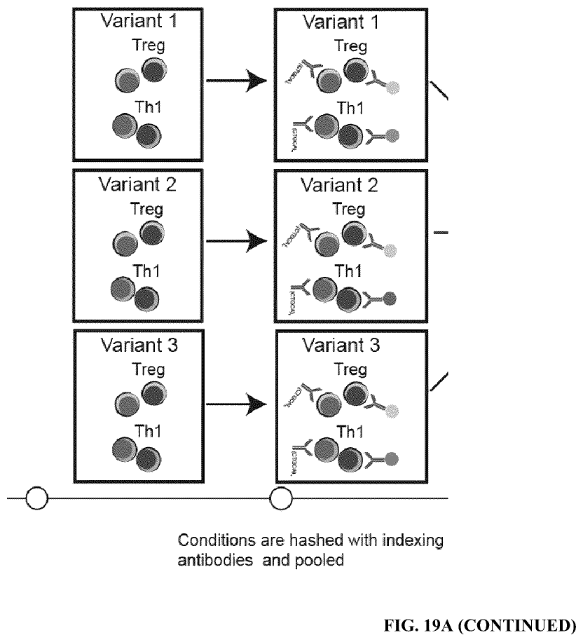

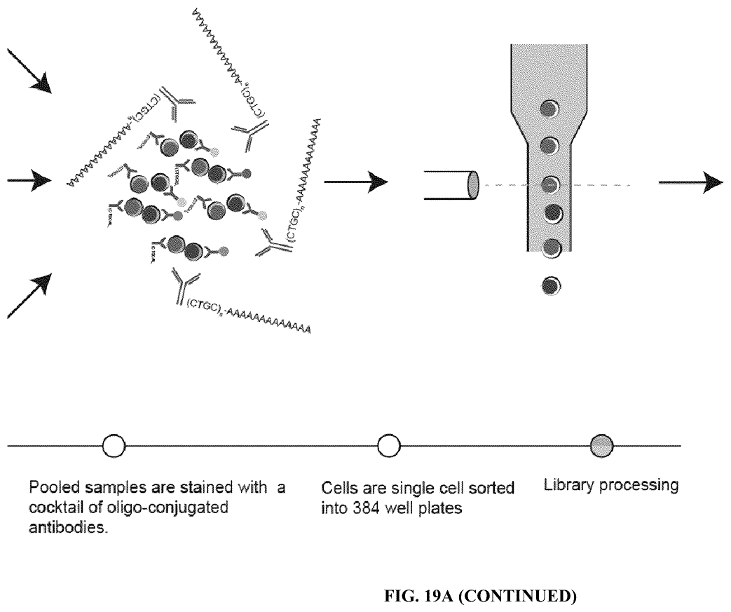

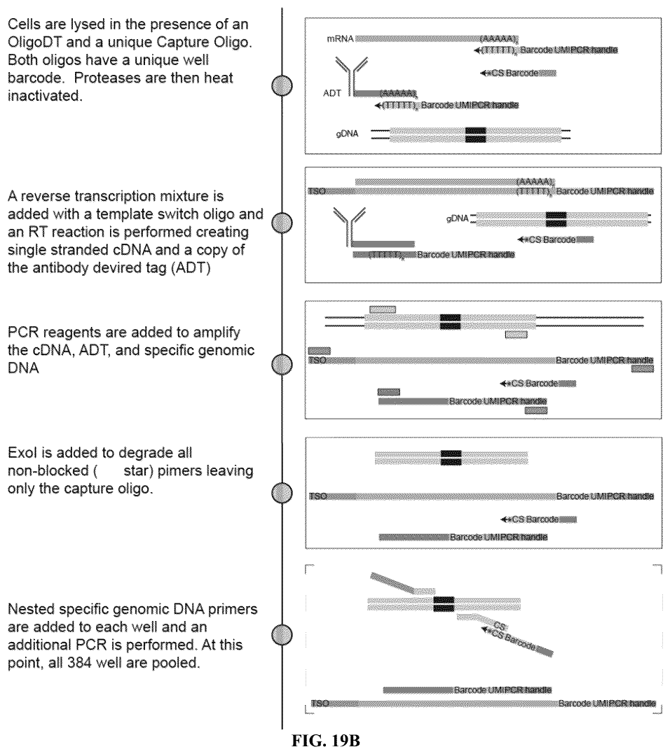

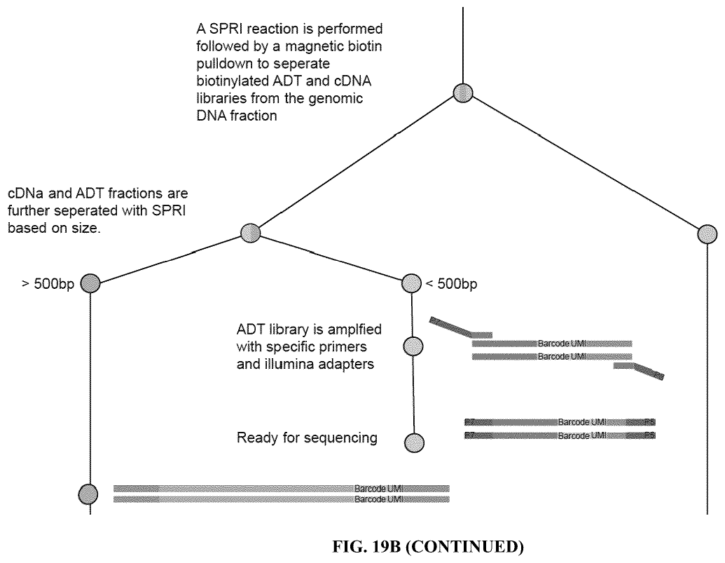

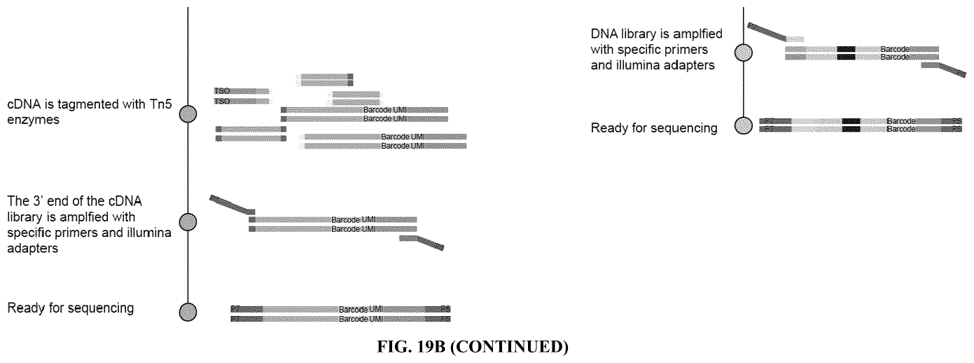

In some experiments, a MINECRAFTseq was carried out as described in FIGS. 19A and 19B. In the MINECRAFTseq method described in FIGS. 19A and 19B, cells are lysed in the presence of a capture oligomer (capture oligo.) comprising a capture sequence (CS) and a well-specific barcode. The cells are also lysed in the presence of an OligoDT primer. The capture oligomer comprises a blocking agent that prevents degradation of the capture oligomer by ExoI. After amplification of the cDNA, ADT, and specific genomic DNA, non-blocked single-stranded DNA oligomers are digested using ExoI. After the ExoI digestion, nested specific genomic DNA primers are added to each well and an additional PCR is performed. One of the specific DNA primers comprises the capture sequence so that the capture sequence is added to the amplification product, which results in the capture oligomer ultimately being ligated to amplicons produced using the nested specific genomic DNA primers. This modified version of the MINECRAFTseq method has the advantage of allowing all PCR amplifications subsequent to library preparation to be carried out in a single well, thereby simplifying the MINECRAFTseq method. Non-limiting examples of blocking agents include phosphoryl and acetyl groups. In embodiments, the blocking agent is covalently linked to the 3′OH group of the capture oligomer. In embodiments, the capture sequence is a unique sequence that occurs in the genome of a target cell less than 1, 2, 3, 4, 5, 6, 7, 8, 9, 10, 15, 20, or 100 times.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method for concurrently characterizing single cell genomic DNA and mRNA, the method comprising
(a) labelling a plurality of isolated cells with a detectable antibody that specifically binds a cell surface marker of interest;
(b) incubating the detectably labelled cells of (a) with an oligo-conjugated antibody;
(c) index sorting the cells into single wells, characterizing the cell surface marker expression of each cell, and lysing the cells in the presence of dNTPs, a well-specific barcoded oligoDT primer comprising a unique molecular identifier (UMI), and a PCR handle;
(d) incubating the product of (c) with reverse transcriptase, a custom template switch oligo (TSO) comprising one member of a binding pair, under conditions that permit generation of cDNA;
(e) incubating the product of step (d) with genomic primers that specifically bind a region of interest (ROI), cDNA amplification primers that specifically bind the PCR handle and the TSO, an antibody derived tag (ADT) specific primer, dNTPs, and a polymerase under conditions that support amplification, thereby simultaneously amplifying gDNA, cDNA, and ADT to form cDNA, genomic ROI, and ADT libraries;
(f) incubating at least a portion of the genomic DNA from each well of (e) with dNTPs, polymerase, and nested primers that specifically bind a region of interest to obtain a gDNA library, wherein at least one of the nested primers comprises:
i) a well-specific barcode, a UMI, and a PCR handle; or
ii) a capture sequence wherein when the nested primers comprise the capture sequence, the step further comprises incubating the product of (e) with an exonuclease and a capture oligo, wherein the capture oligo comprises the capture sequence, a well-specific barcode, an exonuclease blocking agent, and a UMI, wherein the capture oligo binds to an amplicon produced using the nested primers effectively labeling the product with the barcode during the PCR reaction,
(g) pooling at least a portion of a sample from each well after step (e) or step (f), and subsequently separating at least two of the cDNA, ADT libraries, and gDNA libraries; and
(h) preparing the gDNA, cDNA, and ADT libraries for sequencing by amplifying each library in the presence of sequencing primers.

2. A method for concurrently characterizing DNA amplicons, 3′ mRNA transcripts, antibody derived tags (ADT), and index flow sorting information from a cell sample, the method comprising
(a) labelling a plurality of cells with a detectable antibody that specifically binds a cell surface marker of interest and single-cell index sorting said cells into individual wells;
(b) lysing the cells in the presence of a reverse transcriptase, a template switch oligo, well-specific barcodes, a primer comprising an oligoDT primer comprising a unique molecular identifier (UMI), and a PCR handle, and ADTs under conditions that permit reverse transcription to obtain cDNA;
(c) amplifying the cDNA, ADT, and specific genomic DNA in a single pool comprising genomic primers that specifically bind a region of interest, cDNA amplification primers that specifically bind the PCR handle and the TSO, an ADT specific primer, dNTPs, and a Taq polymerase, thereby simultaneously amplifying gDNA, cDNA, and ADT to form cDNA, genomic ROI, and ADT libraries;
(d) at least a portion of the product of (c) is used for further amplification of the genomic ROI with nested primers to obtain a gDNA library, wherein at least one of the nested primers comprise:
i) a well-specific barcode, a UMI, and a PCR handle; or
ii) a capture sequence, wherein when the nested primers comprise the capture sequence, the step further comprises incubating the product of (c) with an exonuclease and a capture oligo, where the capture oligo comprising the capture sequence, a well-specific barcode, an exonuclease blocking agent, and a UMI, wherein the capture oligo binds to an amplicon produced using the nested primers effectively labeling the product with the barcode during the PCR reaction; (e) pooling at least a portion of each well and subsequently separating at least two of the gDNA, cDNA, and ADT libraries,
(f) preparing the gDNA, cDNA, and ADT libraries for sequencing, wherein preparing the libraries for sequencing comprises amplifying the ADT library with sequencing primers, tagmenting the cDNA library and preferentially amplifying the 3′ ends with sequencing primers, and amplifying the gDNA library using sequencing primers.

3. The method of claim 1, further comprising sequencing the libraries.

4. The method of claim 1, further comprising adding the capture oligo prior to amplification of the gDNA, cDNA, and ADT for the first time.

5. The method of claim 1, wherein the exonuclease is ExoI.

6. The method of claim 1, wherein the blocking agent is a phosphoryl or acetyl group.

7. The method of claim 6, wherein the blocking agent is linked to the 3′OH group of the capture oligomer.

8. The method of claim 1, wherein all amplifications prior to preparing the gDNA, cDNA, and ADT libraries are carried out in the same well.

9. The method of claim 1, wherein formation of the cDNA, genomic ROI, and ADT libraries is carried out in a first well and the gDNA library is prepared in a separate well.

10. The method of claim 1, wherein the gDNA, cDNA, and/or ADT libraries are separated using Solid Phase Reversible Immobilization beads (SPRI) beads.

11. The method of claim 10, wherein the separation involves first separating the gDNA library from the cDNA and ADT libraries using SPRI beads and subsequently separating the cDNA library from the ADT library using SPRI beads.

12. The method of claim 11, wherein the separation of the cDNA library from the ADT library involves separating from one another amplicons that are greater than 500 bp in length and amplicons that are less than 500 bp in length, respectively.

13. The method of claim 1, wherein separation of the cDNA and ADT libraries is carried out prior to or in parallel with preparation of the gDNA library.

14. The method of claim 1, wherein one or more of the cells comprises an alteration in a genomic DNA sequence relative to the sequence of a reference genome.

15. The method of claim 4, wherein the alteration was introduced using a genomic editing technique.

16. The method of claim 15, wherein the genomic editing technique involves base-editing or homology-directed recombination (HDR) editing.

17. The method of claim 1, wherein the cell surface marker is CD45, CD81, or MHC class 1.

18. A method for concurrently characterizing single cell genomic DNA and mRNA, the method comprising
(a) labelling a plurality of isolated cells with a detectable antibody that specifically binds a cell surface marker of interest;
(b) incubating the detectably labelled cells of (a) with an oligo-conjugated antibody;
(c) index sorting the cells into single wells, characterizing the cell surface marker expression of each cell, and lysing the cells in the presence of dNTPs, a well-specific barcoded oligoDT primer comprising a unique molecular identifier (UMI), and a PCR handle, and a capture oligo comprising a capture sequence, a well-specific barcode, an exonuclease blocking agent, and a unique molecular identifier;
(d) incubating the product of (c) with reverse transcriptase, a custom template switch oligo (TSO) comprising one member of a binding pair, and a reverse transcriptase under conditions that permit generation of cDNA;
(e) incubating the product of step (d) with genomic primers that specifically bind a region of interest (ROI), cDNA amplification primers that specifically bind the PCR handle and the TSO, an antibody derived tag (ADT) specific primer, dNTPs, and a polymerase under conditions that support amplification, thereby simultaneously amplifying gDNA, cDNA, and ADT to form cDNA, genomic ROI, and ADT libraries;
(f) contacting the product of step (e) with an exonuclease to degrade unconsumed primers;
(g) incubating at least a portion of the genomic ROI libraries from each well of (f) with dNTPs, polymerase, and nested primers capable of specific amplification of a region within the genomic ROI library, wherein at least one of the nested primers comprises the capture sequence, wherein the capture oligo binds to an amplicon produced using the nested primers effectively labeling the product with the barcode during the PCR reaction, and obtaining a gDNA library,
(g) pooling at least a portion of a sample from each well, and subsequently separating the gDNA, cDNA, and ADT libraries; and
(h) preparing the gDNA, cDNA, and ADT libraries for sequencing by amplifying each library in the presence of sequencing primers.

19. The method of claim 18, wherein the exonuclease blocking agent is a phosphoryl or an acetyl group.

20. A method for concurrently characterizing single cell genomic DNA and mRNA, the method comprising
(a) labelling a plurality of isolated cells with a detectable antibody that specifically binds a cell surface marker of interest;
(b) incubating the detectably labelled cells of (a) with an oligo-conjugated antibody;
(c) index sorting the cells into single wells, characterizing the cell surface marker expression of each cell, and lysing the cells in the presence of dNTPs, and a well-specific barcoded oligoDT primer comprising a unique molecular identifier (UMI), and a PCR handle;
(d) incubating the product of (c) with reverse transcriptase, and a custom template switch oligo (TSO) comprising one member of a binding pair, under conditions that permit generation of cDNA;
(e) incubating the product of step (d) with genomic primers that specifically bind a region of interest (ROI), cDNA amplification primers that specifically bind the PCR handle and the TSO, an antibody derived tag (ADT) specific primer, dNTPs, and a polymerase under conditions that support amplification, thereby simultaneously amplifying gDNA, cDNA, and ADT to form cDNA, genomic ROI, and ADT libraries;
(f) pooling at least a portion of a sample from each well, and subsequently separating the cDNA and ADT libraries;
(g) incubating at least a portion of the genomic DNA from each well of (e) with dNTPs, polymerase, and nested primers that specifically bind a region of interest, wherein the nested primers comprise a well-specific barcode, a UMI, and a PCR handle, to obtain a gDNA library, and
(h) preparing the gDNA, cDNA, and ADT libraries for sequencing by amplifying each library in the presence of sequencing primers.


SEQUENCE LISTING (15 sequences)

SEQ ID NO: 1 - DNA, 12 bp, synthetic construct
  misc_feature 1..12: note = Description of Artificial Sequence: Synthetic oligonucleotide
  source 1..12: mol_type = other DNA
ctggttctgttg

SEQ ID NO: 2 - DNA, 12 bp, synthetic construct
  misc_feature 1..12: note = Description of Artificial Sequence: Synthetic oligonucleotide
  source 1..12: mol_type = other DNA
gctactccagtt

SEQ ID NO: 3 - DNA, 13 bp, synthetic construct
  misc_feature 1..13: note = Description of Artificial Sequence: Synthetic oligonucleotide
  source 1..13: mol_type = other DNA
ttttttttttttt

SEQ ID NO: 4 - DNA, 17 bp, synthetic construct
  misc_feature 1..17: note = Description of Artificial Sequence: Synthetic oligonucleotide
  misc_feature 1..17: note = See specification as filed for detailed description of substitutions and preferred embodiments
  source 1..17: mol_type = other DNA
ctgcaaaaaaaaaaaaa

SEQ ID NO: 5 - DNA, 40 bp, Homo sapiens
  source 1..40: mol_type = other DNA
agtgaagctgcacatcaaggagtaattacctggaatcccc

SEQ ID NO: 6 - DNA, 60 bp, Homo sapiens
  source 1..60: mol_type = other DNA
ggtcctgccaggggctctcaggtcagtctgagcagagcagctggcatcaccaaagcagcc

SEQ ID NO: 7 - DNA, 60 bp, Homo sapiens
  source 1..60: mol_type = other DNA
aacggctttttgtttaatcgtagagtttcaagaagaaaactctttaggcaacaaaactat

SEQ ID NO: 8 - DNA, 40 bp, Homo sapiens
  source 1..40: mol_type = other DNA
ctgaaggaggtatctattttggtcccaaacagaaaagagt

SEQ ID NO: 9 - DNA, 40 bp, Homo sapiens
  source 1..40: mol_type = other DNA
tccctggaatgtcactgatggaaatttaccagtccctcag

SEQ ID NO: 10 - DNA, 40 bp, Homo sapiens
  source 1..40: mol_type = other DNA
tggggtctctctccctggaatctcattgatggaaatttac

SEQ ID NO: 11 - DNA, 60 bp, Homo sapiens
  source 1..60: mol_type = other DNA
cccactcttttctgtttgggaccaaaatagatacctccttcagaagccgctgctttctgt

SEQ ID NO: 12 - DNA, 60 bp, Homo sapiens
  source 1..60: mol_type = other DNA
cccacccaaatagcagcattcggcatcatgtggtgggttcctcatggacgcccgctgttt

SEQ ID NO: 13 - DNA, 60 bp, Homo sapiens
  source 1..60: mol_type = other DNA
aagctgccttgtctgtaatgcctggacggtgcccggtggccgccctgcacatgcctgacc

SEQ ID NO: 14 - DNA, 40 bp, Homo sapiens
  source 1..40: mol_type = other DNA
tccctggaatgtcattgatggaaatttaccagtccctcag

SEQ ID NO: 15 - DNA, 40 bp, Homo sapiens
  source 1..40: mol_type = other DNA
tggggtctctctccctggaatctcactgatggaaatttac